SEQUENCE LISTING (31 sequences)

SEQ ID NO: 1 - DNA, 17 bp, Artificial Sequence
  ACTB 5' primer
acagagcctcgcctttg

SEQ ID NO: 2 - DNA, 17 bp, Artificial Sequence
  ACTB 3' primer
ccttgcacatgccggag

SEQ ID NO: 3 - DNA, 23 bp, Artificial Sequence
  B2M 5' primer
ggactggtctttctatctcttgt

SEQ ID NO: 4 - DNA, 20 bp, Artificial Sequence
  B2M 3' primer
acctccatgatgctgcttac

SEQ ID NO: 5 - DNA, 23 bp, Artificial Sequence
  CDH5 5' primer
gaaccagatgcacattgatgaag

SEQ ID NO: 6 - DNA, 22 bp, Artificial Sequence
  CDH5 3' primer
tgcccacatattctcctttgag

SEQ ID NO: 7 - DNA, 22 bp, Artificial Sequence
  PAX6 5' primer
atttcagagccccatattcgag

SEQ ID NO: 8 - DNA, 19 bp, Artificial Sequence
  PAX6 3' primer
gacaccaccgagctgattc

SEQ ID NO: 9 - DNA, 22 bp, Artificial Sequence
  PECAM1 5' primer
aacagtgttgacatgaagagcc

SEQ ID NO: 10 - DNA, 22 bp, Artificial Sequence
  PECAM1 3' primer
tgtaaaacagcacgtcatcctt

SEQ ID NO: 11 - DNA, 18 bp, Artificial Sequence
  POLR2A 5' primer
gagcaatcggcctgtcat

SEQ ID NO: 12 - DNA, 22 bp, Artificial Sequence
  POLR2A 3' primer
acatcaggaggttcatcacttc

SEQ ID NO: 13 - DNA, 21 bp, Artificial Sequence
  POU5F1 5' primer
gttggagggaaggtgaagttc

SEQ ID NO: 14 - DNA, 22 bp, Artificial Sequence
  POU5F1 3' primer
tgtgtctatctactgtgtccca

SEQ ID NO: 15 - DNA, 20 bp, Artificial Sequence
  RBFOX3 5' primer
cggtcgtgtatcaggatgga

SEQ ID NO: 16 - DNA, 19 bp, Artificial Sequence
  RBFOX3 3' primer
gccgtaactgtcgctgtag

SEQ ID NO: 17 - DNA, 21 bp, Artificial Sequence
  SOX2 5' primer
gtacaactccatgaccagctc

SEQ ID NO: 18 - DNA, 18 bp, Artificial Sequence
  SOX2 3' primer
cttgaccaccgaacccat

SEQ ID NO: 19 - DNA, 21 bp, Artificial Sequence
  TUBB3 5' primer
gttctgggaagtcatcagtga

SEQ ID NO: 20 - DNA, 19 bp, Artificial Sequence
  TUBB3 3' primer
ctcgaggcacgtacttgtg

SEQ ID NO: 21 - DNA, 21 bp, Artificial Sequence
  ACTB probe
  misc_feature (1)..(1): 56-FAM label
  misc_feature (9)..(9): ZEN label
  misc_feature (21)..(21): 3IABkFQ label
tcatccatggtgagctggcgg

SEQ ID NO: 22 - DNA, 23 bp, Artificial Sequence
  B2M probe
  misc_feature (1)..(1): 56-FAM label
  misc_feature (9)..(9): ZEN label
  misc_feature (23)..(23): 3IABkFQ label
cctgccgtgtgaaccatgtgact

SEQ ID NO: 23 - DNA, 25 bp, Artificial Sequence
  CDH5 probe
  misc_feature (1)..(1): 56-FAM label
  misc_feature (9)..(9): ZEN label
  misc_feature (25)..(25): 3IABkFQ label
tgagtcgcaagaatgccaagtacct

SEQ ID NO: 24 - DNA, 23 bp, Artificial Sequence
  PAX6 probe
  misc_feature (1)..(1): 56-FAM label
  misc_feature (9)..(9): ZEN label
  misc_feature (23)..(32): 3IABkFQ label
cagcatgcagaacagtcacagcg

SEQ ID NO: 25 - DNA, 24 bp, Artificial Sequence
  PECAM1 probe
  misc_feature (1)..(1): 56-FAM label
  misc_feature (9)..(9): ZEN label
  misc_feature (24)..(24): 3IABkFQ label
aaatgggaagaacctgaccctgca

SEQ ID NO: 26 - DNA, 22 bp, Artificial Sequence
  POLR2A probe
  misc_feature (1)..(1): 56-FAM label
  misc_feature (9)..(9): ZEN label
  misc_feature (22)..(22): 3IABkFQ label
actgctgtgagtgtgtcctgca

SEQ ID NO: 27 - DNA, 34 bp, Artificial Sequence
  POU5F1 probe
  misc_feature (1)..(1): 56-FAM label
  misc_feature (9)..(9): ZEN label
  misc_feature (34)..(34): 3IABkFQ label
tgctcttgattttaatcccacatcatgtatcact

SEQ ID NO: 28 - DNA, 27 bp, Artificial Sequence
  RBFOX3 probe
  misc_feature (1)..(1): 56-FAM label
  misc_feature (9)..(9): ZEN label
  misc_feature (27)..(27): 3IABkFQ label
atggtgctgagatttatggaggctacg

SEQ ID NO: 29 - DNA, 24 bp, Artificial Sequence
  SOC2 probe
  misc_feature (1)..(1): 56-FAM label
  misc_feature (9)..(9): ZEN label
  misc_feature (24)..(24): 3IABkFQ label
cacctacagcatgtcctactcgca

SEQ ID NO: 30 - DNA, 21 bp, Artificial Sequence
  TUBB3 probe
  misc_feature (1)..(1): 56-FAM label
  misc_feature (9)..(9): ZEN label
  misc_feature (21)..(21): 3IABkFQ label
atccgctccagctgcaagtcc

SEQ ID NO: 31 - DNA, 20 bp, Artificial Sequence
  20bp barcode from Gateway LR Clonase II Enzyme mix
caaagtgaaaccagagtcgc

(19) United States
(12) Patent Application Publication
(10) Pub. No.: US 2023/0287357 A1
(43) Pub. Date: Sep. 14, 2023

(54) PROGRAMMABLE ORGANOIDS AND METHODS OF PRODUCING THE SAME VIA ORTHOGONAL DIFFERENTIATION AND BIOPRINTING

(71) Applicant: President and Fellows of Harvard College, Cambridge, MA (US)

(72) Inventors: Mark Andrew SKYLAR-SCOTT, Palo Alto, CA (US); Jennifer A. LEWIS, Cambridge, MA (US); Jeremy Yuenchen HUANG, Cambridge, MA (US); Jingcheng LU, Somerville, MA (US); Tomoya DUENKI, Dachsen (CH)

(73) Assignee: President and Fellows of Harvard College, Cambridge, MA (US)

(21) Appl. No.: 18/012,064

(22) PCT Filed: Jul. 6, 2021
(86) PCT No.: PCT/US2021/040519

§ 371 Date: Dec. 21, 2022

Related U.S. Application Data
(60) Provisional application No. 63/048,502, filed on Jul. 6, 2020.

(51) Int. Cl. C12N 5/071; C12N 15/63; C12N 15/85; B33Y 10/00; B33Y 80/00; B29C 64/118

(52) U.S. Cl. CPC C12N 5/0697; B29C 64/118; B33Y 10/00; B33Y 80/00; C12N 15/85; C12N 15/635; B29L 2031/7532; C12N 2506/45; C12N 2513/00; C12N 2533/54; C12N 2533/56; C12N 2800/90; C12N 2800/107

(57) ABSTRACT

Described herein are methods of generating a programmable multicellular organoid and/or a 3D organ-specific tissue. Also, described are the programmable multicellular organoid and/or a 3D organ-specific tissue produced by the described methods. Also, described herein are in vitro methods of generating functional human tissue construct.

### RELATED APPLICATIONS

The present patent document claims the benefit of the filing date under 35 U.S.C. §119(e) of Provisional U.S. Pat. Application Serial No. 63/048,502, filed Jul. 6, 2020, which is hereby incorporated by reference.

### FEDERALLY SPONSORED RESEARCH OR DEVELOPMENT

This invention was made with Government support under Grant No. I RMl HG008525, awarded by the National Institute of Health (NIH). The Government has certain rights in this invention.

### REFERENCE TO APPENDIX [CD ROM/SEQUENCE LISTING]

This application is being filed electronically via EFS-Web and includes an electronically submitted Sequence Listing in .txt format. The .txt file contains a sequence listing entitled “14968_00219_Seq_Listing_ST25” created on Jul. 1, 2021 and is 8,201 bytes in size. The sequence listing contained in this .txt file is part of the specification and hereby incorporated by reference in its entirety.

### BACKGROUND

PCT Pub. Nos. WO2016141137A1 and WO2016141137A1 are hereby incorporated by reference in their entireties.

Recent innovations in organoid development and 3-dimensional (3D) bioprinting offer emerging pathways to creating autologous human tissues for drug screening and therapeutic applications¹⁻⁴. The ability to generate organoids via self-assembly and differentiation of embryoid bodies (EBs), which are aggregated from human induced pluripotent stem cells (hiPSCs), provides a bottom-up approach to creating organ-like microarchitectures^{5,6}. By contrast, multimaterial 3D bioprinting offers a top-down method for fabricating heterogeneous stem-cell derived tissues⁷⁻¹². However, both techniques are limited by the speed, efficiency, and scalability of stem cell differentiation. While protocols for generating cerebral¹³⁻¹⁵, renal¹⁶⁻¹⁸, retinal¹⁹ and other organoids have recently been reported, they often rely on prolonged culture times ranging from weeks to several months to approach organ-level cellular diversity.

Moreover, organoid protocols that generate a wider range of cells and tissues generally result in less reproducible organoids, giving rise to a trade-off between organoid reproducibility and cellular diversity^{20,21}. A similarly large number of cell types, each differentiated and rendered into densely cellular bioinks, would be required for multimaterial 3D bioprinting of organ-specific tissues. Traditional organoid differentiation protocols typically aim to derive cells from a single germ layer^{13-15,20,21}.

Hence, new approaches that enhance the specificity, efficiency, and scalability of stem cell differentiation are needed to generate programmable multicellular organoids and tissues from pluripotent stem cells. However, the generation of more complex multicellular tissues requires not only a broader range of programmable cell types, but methods that simultaneously enable control over their spatial patterning.

New methods of creating embryoid bodies or organoids and tissue constructs suitable for studies of tissue development and disease, as well as transplantation are desired.

### FIGURES

The patent or application file contains at least one drawing executed in color. Copies of this patent or patent application publication with color drawing(s) will be provided by the Office upon request and payment of the necessary fee.

FIG. 1 shows an illustration of orthogonally induced differentiation platform for genomically programming stem cells (a), organoids (b), and 3D bioprinted organ-specific tissues (c).

FIG. 2 shows programmable differentiation of pluripotent stem cells via orthogonally induced differentiation under identical media conditions: (a) Left, schematic detailing wild-type (WT) hiPSC differentiation into neural stem cells in NIM. Right, immunostaining of Oct4 and F-actin of WT colonies on day 0; (b), Left, schematic detailing iEndo differentiation through doxycycline-induced ETV2 isoform-2 overexpression. Right, immunostaining of Oct 4 and F-actin of iEndo colonies on day 0; (c), Left, schematic detailing iNeuron differentiation through doxycycline-induced NGNI overexpression in culture. Right, immunostaining of Oct4 and F-actin of iNeuron colonies on day 0. (d), WT hiPSCs cultured in NIM for 6 days without (left column) or with (right column) doxycycline. Immunostaining of N-cadherin and Pax6, ZOI and Sox2, and nestin; (e), iEndo cells cultured in NIM for 6 days without (left column) or with (right column) doxycycline. Left, immunostaining of N-cadherin and Pax6, ZOI and Sox2, and nestin. Right, immunostaining of vWF and VE-cadherin, CD31, and Nrpl; (f), iNeuron cells cultured in NIM for 6 days without (left column) or with (right column) doxycycline. Left, immunostaining of N-cadherin and Pax6, ZOI and Sox2, and nestin. Right, immunostaining of TuJI, MAP2, and NeuN; (g), Flow cytometry plots quantifying WT hiPSC differentiation to Pax6+ neural stem cells by 6 days with (grey) and without (blue) doxycycline; (h), Quantification of populations; (I), Flow cytometry plots indicating that, in the presence of doxycycline, iEndos differentiate into VECad+ endothelium (red). In the absence of doxycycline, iEndos differentiate into Pax6+ neural stem cells (blue); (j), Quantification of populations, mean ± s.e.m., n = 3 biological replicates, **** P = 7.06× 10⁻⁷, unpaired two-tailed t-test; (k), Flow cytometry plots indicating that, in the presence of doxycycline, iNeurons differentiate into MAP2+ neurons (green). In the absence of doxycycline, iNeurons differentiate into Pax6+ neural stem cells (blue); (I), quantification of populations, mean ± s.e.m., n = 3 biological replicates, **** P = 3.53× 10⁻⁵, unpaired two-tailed t-test. Scale bars: 50 µm in a-f.

FIG. 3 shows programmable vascularization of cortical organOIDs: (a), Schematic of the vascularized cortical organoid protocol; (b), Fluorescent images of hiPSCs in microwells three days before suspension culture. Top, 100% WT-eGFP hiPSCs. Bottom, 67% WT-eGFP and 33% iEndo-mKate2 randomly pooled hiPSCs; (c), Fluorescent images of resulting EBs. Top, 100% WT-eGFP EBs. Bottom, 67% WT-eGFP and 33% iEndo-mKate2 randomly pooled EBs; (d), Brightfield images of organoids derived from EBs cultured for 10 days. Top, 100% WT-eGFP organoids. Bottom, 67% WT-eGFP and 33% iEndo-mKate2 pooled organoids; (e), Immunostaining of Sox2 and UEAI labeling of organoids cultured for 10 days. Top, 100% WT organoids. Bottom, 67% WT and 33% iEndo pooled organOIDs; (f), Left, top, confocal maximum intensity z-projections obtained from immunostaining of Sox2 and nestin of 67% WT and 33% iEndo-mKate2 pooled organOIDs cultured for 10 days; right column, individual channels for Sox2, nestin, and mKate2. Arrowheads indicate Sox2+ positive cells that co-localize with the vasculature. Left, bottom, immunostaining of Sox2 and CD31 with iEndo-mKate2 of 67% WT and 33% iEndo-mKate2 pooled organOIDs cultured for 10 days; (g), Immunostaining of Sox2, NeuN, and CD31 of organoids cultured for 25 days. Top, 100% WT organoids. Bottom, 67% WT and 33% iEndo pooled organOIDs; (h), Immunostaining of Sox2 and CD31 of organoids cultured for 25 days. Top, 100% WT organoids. Bottom, 67% WT and 33% iEndo pooled organOIDs; (i), Left, maximum intensity z-projection with immunostaining of Sox2 and CD31 of 67% WT and 33% iEndo pooled organOIDs cultured for 25 days. Right, optical slices of Sox2 and CD31 channels at depths of z = 0 µm and z = 335 µm; (j), Quantification of vascular area in slices of a single 100% WT organoid and a single 67% WT and 33% iEndo pooled organOIDs. Data represents mean ± s.e.m. (n = 5, unpaired two-tailed t-test) **** P = 4.23× 10⁻⁵; (k), Maximum intensity z-projection with immunostaining of Sox2, MAP2, and VE-Cadherin of 67% WT and 33% iEndo pooled organOIDs cultured for 45 days; (I), RT-qPCR log fold-change expression bar-plot of 67% WT and 33% iEndo pooled organOID versus 100% WT organoid. Data represents mean ± s.e.m. (n = 6, from three independent batches). Scale bars: 200 µm in d, f, h, i, and k; 100 µm in b, c, e, and g.

FIG. 4 shows multicore-shell cortical organOIDs: (a), Schematic of the pooled and multicore-shell organoid formation protocols; (b), Quantification of hiPSCs seeding distribution of WT, iEndo, and iNeurons in WT-only, pooled, and multicore-shell approaches. Data represents the mean ± s.e.m. (n = 3, from three independent batches); (c), WT-only organoids cultured for 1 day. Left, fluorescence image of CellTracker labeled WT-only organoids; right, immunostaining for Oct4; (d), Randomly pooled organOIDs cultured for 1 day. Left, fluorescence image of CellTracker labeled randomly pooled organoids; right, immunostaining for Oct4; (e), Multicore-shell organOIDs cultured for 1 day after final shell seeding. Left, fluorescence image of CellTracker labeled multicore-shell organOID; right, immunostaining for Oct4. Left-bottom, immunostaining for Oct4; (f), Immunostaining of Sox2, NeuN, and CD31 of WT-only organoids cultured for 10 days; (g), Immunostaining of Sox2, NeuN, and CD31 of pooled organOIDs cultured for 10 days; (h), Immunostaining of Sox2, NeuN, and CD31 of multicore-shell organOIDs cultured for 10 days; (i), UMAP plot of combined cellular composition of day 25 organoids; (j), Top, UMAP plots of WT, random, and multicore-shell organoids; (k), UMAP plots highlighting endothelial and barcoded iNeuron populations; (I), total number of cells in cluster 7 for WT, random, and multicore-shell organoids. Data represents mean ± s.e.m (n = 4, from two independent batches); (m), heatmap comparison of clusters 0, 3, and 7 for common endothelial and stromal cell markers; (n), heatmap comparison of clusters 1, 2, 5, 6, and barcoded iNeuron cells for common neuronal and neural stem cell markers. Scale bars: 100 µm in c-h.

FIG. 5 shows multicellular neural tissues via 3D bioprinting coupled with orthogonal induced differentiation: (a), Schematic of the bioprinting and gel casting process; (b), Image of bioink extrusion through 50 µm nozzle during bioprinting process; (c), Brightfield images of bioprinted filamentary features; (d), Total cell number and flow-cytometry quantification of Oct4+ cells within bioprinted filaments immediately after printing. Data represents mean ± s.d. (n = 3 from three independent cell ink batches); (e), hiPSC viability within bioprinted filaments at different speeds and casted (control) samples, as measured using calcein-AM/ethidium homodimer live/dead assays. Data represents mean ± s.e.m. (n = 3, from three independent batches); (f), Live/dead staining of bioprinted filaments produced using nozzle diameters of 100 µm (top, left) and 50 µm (top, right) and the corresponding distributions (bottom) of their fluorescence intensity; (g), Immunostaining of Oct4 in patterns fixed on day 0, immediately after printing; (h), Bioprinted tissue architectures cultured in NIM. Left, immunostaining of N-cadherin and Sox2 for WT printed patterns at day 4. Middle, immunostaining of VE-cadherin for iEndo printed patterns at day 6. Right, immunostaining of TuJI and NeuN of iNeuron printed patterns at day 6; (i), Design of triple-nozzle for multimaterial bioprinting; (j), Fluorescence image of bioprinted CellTracker labeled WT, iEndo, and iNeuron inks; (k), Immunostaining of TuJI, UEAI and Sox2 of 3D printed multicellular tissues at day 6. Scale bars: 500 µm in g (top) and h (top); 200 µm in c (left) and i-k; 50 µm in c (right), g (bottom), and h (bottom).

FIG. 6 shows flow cytometry gating examples: Gating strategies for (a) WT PGPI cells, (b) iEndo cells, and (c) iNeuron cells. Left, forward-scatter (area) vs side-scatter (area). Middle, forward-scatter (width) vs forward-scatter (height). Right, side-scatter (width) vs side-scatter (height).

FIG. 7 shows induced differentiation of stem cells in endothelial differentiation conditions: (a), WT PGPI cells after 8 days of differentiation in endothelial differentiation media conditions without (left column) or with (right column) doxycycline. Immunostaining of VECad and vWF, CD31, and NRPI; (b), iNeuron cells in endothelial differentiation conditions without (left column) or with (right column) doxycycline. Left, immunostaining of VECad and vWF, CD31, and NRPI. Right, immunostaining of TujI, MAP2, and NeuN. Scale-bars: 50 µm in a-b.

FIG. 8 shows orthogonally induced differentiation of pooled programmable hiPSCs form tailorable multi-lineage cultures: (a), Immunostaining of Sox2 and VE-Cadherin of different seeded proportions of WT and iEndo hiPSCs on Matrigel hydrogels cultured in NIM for 6 days. Left, 100% WT culture. Middle-left, 67% WT and 33% iEndo. Middle-right, 33% WT and 67% iEndo. Right, 100% iEndo; (b), Immunostaining of VE-Cadherin and MAP2 of different seeded proportions of iEndo and iNeuron on Matrigel hydrogels cultured in NIM for 6 days. Left, 100% iEndo. Middle-left, 67% iEndo and 33% iNeuron. Middle-right 33% iEndo and 67% iNeuron. Right, 100% iNeuron; (c), Immunostaining of Map2 and Sox2 of different seeded proportions of WT and iNeuron cells on Matrigel hydrogels cultured in NIM for 6 days. Left, 100% iNeuron. Middle-left, 67% iNeuron and 33% WT. Middle-right, 33% iNeuron and 67% WT. Right, 100% WT; (d), Without doxycycline, immunostaining of Sox2, Map2, and VE-Cadherin of 33% WT, 33% iEndo, and 33% iNeuron on Matrigel hydrogel cultured in NIM for 6 days; (e), With doxycycline, immunostaining of Sox2, Map2, and VE-Cadherin of 33% WT, 33% iEndo, and 33% iNeuron on Matrigel hydrogels cultured in NIM for 6 days. Scale bars: 100 µm in a-e.

FIG. 9 shows that pooled hiPSCs enable the formation of cohesive embryoid bodies with tailorable cellular composition: (a), Left, 50% WT-eGFP and 50% RFP HUVEC EBs in microwell arrays cultured for 1 day. Right, 50% WT-eGFP and 50% RFP HUVEC EBs cultured for 3 days; (b), Left, 50% WT-eGFP and 50% iEndo-mKate2 EBs in microwell arrays cultured for 1 day. Right, WT-eGFP and 50% iEndo-mKate2 EBs cultured for 3 days; (c), Different seeded proportions of WT-eGFP and iEndo-mKate2 hiPSC aggregates in microwell arrays 3 days before suspension culture. Left, 100% iEndo-mKate2. Middle-left, 67% iEndo-mKate2 and 33% WT-eGFP. Middle-right 33% iEndo-mKate2 and 67% WT-eGFP. Right, 100% WT-eGFP; (d), WT-eGFP and iEndo-mKate2 EBs 1 day before suspension culture. Left, 100% iEndo-mKate2. Middle-left, 67% iEndo-mKate2 and 33% WT-eGFP. Middle-right 33% iEndo-mKate2 and 67% WT-eGFP. Right, 100% WT-eGFP. Scale bars: 500 µm in (a) (left), (b) (left), and (c); 100 µm in (a) (right), (b) (right), and (d).

FIG. 10 shows programmable vascularized cortical organOIDs: (a), Immunostaining of Sox2 and nestin in 100% WT organoids cultured for 10 days; (b), Immunostaining of Sox2 and nestin in 67% WT and 33% iEndo organOIDs cultured for 10 days; (c), Immunostaining for Sox2 and UEAI for 67% WT and 33% iEndo organoid cultured for 10 days; (d), Immunostaining of Sox2 and CD31 in 100% WT organoids cultured for 25 days; (e), Immunostaining of Sox2 and CD31 in 67% WT and 33% iEndo organOIDs cultured for 10 days; (f), Immunostaining of Sox2, NeuN, and CD31 for 100% WT organoids cultured for 45 days; (g), Immunostaining for Sox2, NeuN, and CD31 in a 67% WT and 33% iEndo organOIDs cultured for 45 days. Scale bars: 200 µm in a, b, d, f (left), and g (left), 100 µm in c and e, and 50 µm in f (right) and g (right).

FIG. 11 shows quantifying vascularization within programmable cortical organOIDs: Example Angiotool processing workflow for (a), 67% WT and 33% iEndo organOIDs cultured for 25 days; (b), 100% WT organoids cultured for 25 days. Left, Individual z sections taken from a confocal z-stack of iDISCO cleared organoids. Middle, Background is eliminated by increasing the minimum threshold on all images. Right, Angiotool analysis of the corresponding z sections. Red lines represent vascular paths, blue dots represent vascular junctions, yellow lines represent the boundaries of the vasculature, and the thin white line marks the calculated vascularized area. Vessel area is calculated as the total area enclosed by the yellow lines.

FIG. 12 shows large ventricular architectures in programmable multicore-shell cortical organOIDs: (a), Immunostaining of Sox2 in 10-day-old WT-only organoids; (b), Immunostaining of Sox2 in 10 day-old randomly pooled organOIDs; (c), Immunostaining of Sox2 in 10-day-old multicore-shell organOIDs; (d), Immunostaining of Sox2 and N-cadherin in 10-day old WT-only organoids; (e), Immunostaining of Sox2 and N-cadherin in 10-day old randomly pooled organOIDs; (f), Immunostaining of Sox2 and N-cadherin in 10-day old multicore-shell organOIDs; (g), Lengths of neuroepithelium within ventricles as measured by the length of N-cadherin expression within ventricles. Data represents mean ± s.e.m. * P = 0.0169 between WT-only organoids and randomly pooled organOIDs, * P = 0.0286 between randomly pooled and multicore-shell organOIDs (n = 72 WT ventricles, n = 10 randomly pooled ventricles, n = 30 multicore-shell ventricles, unpaired two-tailed t-test). Scale bars: 100 µm in a-f.

FIG. 13 shows singleCellNet classification of cell identities: Input data from annotated primary human brain samples were used to train a classifier, which is then compared against our own single cell cluster datasets. The tool then outputs a classification heatmap that suggests potential cellular identities. 50 random ‘cells’ were generated as control to evaluate classifier fidelity. (a) classification scores for major brain cell types for each cluster. (b) violin plots of each cluster’s classification scores for the most common cell categorizations.

FIG. 14 shows top 10 differentially expressed genes in each cluster for marker-based annotation of cell identities. Seurat 4.0′s FindConservedMarkers function was utilized to find a cluster’s top 10 significantly differentially expressed genes relative to all other clusters for heatmap plotting. Expression levels are z-scored.

FIG. 15 shows that cluster sizes remain consistent across differentiation conditions and batches of organoids: (a) stacked bar plot of cell counts in each cluster from WT, random, and multicore-shell organoids; (b) stacked bar plot of cell counts in each organoid batch from each cluster.

FIG. 16 shows differential gene analysis of WT organoids and randomly pooled organOIDs. Volcano plots showing differentially expressed genes in: (a) cluster 0; (b), cluster I; (c), cluster 2; (d), cluster 3; (e), cluster 4; (f), cluster 5; and (g), cluster 6. Positive log2 fold changes indicate genes more expressed in randomly pooled organoids, negative log2 fold changes indicate genes more expressed in WT organoids. Horizontal blue dashed line marks p_{adj} = 0.05, vertical blue lines mark log2 fold change = -0.5 and 0.5. Genes used in subsequent GO analysis are labeled and marked in blue. Only genes with log2 fold change > 0.25 or < -0.25 are shown.

FIG. 17 depicts differential gene analysis of WT organoids and multicore-shell organOIDs. Volcano plots showing differentially expressed genes in (a) cluster 0; (b) cluster I; (c) cluster 2; (d) cluster 3; (e) cluster 4; (f), cluster 5; and (g), cluster 6. Positive log2 fold changes indicate genes more expressed in multicore-shell organOIDs, negative log2 fold changes indicate genes more expressed in WT organoids. Horizontal blue dashed line marks p_{adj} = 0.05, vertical blue lines mark log2 fold change = -0.5 and 0.5. Genes used in subsequent GO analysis are labeled and marked in blue. Only genes with log2 fold change > 0.25 or < -0.25 are shown.

FIG. 18 depicts differential gene analysis of random and multicore-shell organOIDs. Volcano plots showing differentially expressed genes in (a) cluster 0; (b) cluster I; (c) cluster 2; (d) cluster 3; (e), cluster 4; (f), cluster 5; (g), cluster 6; and h, cluster 7. Positive log2 fold changes indicate genes more expressed in multicore-shell organoids, negative log2 fold changes indicate genes more expressed in randomly pooled organOIDs. Horizontal blue dashed line marks p_{adj} = 0.05, vertical blue lines mark log2 fold change = -0.5 and 0.5. Genes used in subsequent GO analysis are labeled and marked in blue. Only genes with log2 fold change > 0.25 or < -0.25 are shown.

FIG. 19 shows gene ontology analysis of differentially expressed genes in clusters 6 and 7 of randomly pooled and multicore-shell organOIDs: (a) Enriched gene ontology terms of differentially expressed genes in cluster 6 of randomly pooled and multicore-shell organOIDs; (b) Enriched gene ontology terms of differentially expressed genes in cluster 7 of randomly pooled and multicore-shell organOIDs.

FIG. 20 shows multimaterial printhead design for creating 3D multicellular tissue architectures: (a) Perspective view of a 3D CAD model of the triple nozzle; (b) Perspective view of a translucent 3D CAD model of the triple nozzle visualizing the channels inside the printhead; (c) Cross-sectional view and dimensions of the printhead tip of the triple nozzle; and (d) Cross-sectional view of a 3D CAD model of the triple nozzle and the coextrusion of the three bioinks (red, blue, green). Units in c are µm.

FIG. 21 depicts bioprinting of 3D multicellular tissue architectures resembling a germinal zone and overlying neuronal layer: (a) Illustration of a developing fetal human brain section in GWII containing the ventricular zone (shown in blue) and the cortical plate (shown in green); (b) Extracted printing line path of ventricular zone and neuron dense zone from the brain slice; (c), Image of nozzle, bioink, and print surface during brain slice printing; and (d) Brightfield image of printed brain slice structure cultured for 5 days. Scale bar: 1 mm in d.

### SUMMARY

One embodiment relates to a method of generating a programmable multicellular organoid and/or a 3D organ-specific tissue, comprising: culturing at least one genetically-engineered inducible population of stem cells in a cell culture media; concurrently inducing direct differentiation and/or transdifferentiation of the at least one genetically-engineered inducible population of stem cells into at least two divergent populations of the differentiated or transdifferentiated programmable multicellular organoid and/or 3D organ-specific tissue cells, wherein the inducing step is independent of any external cues provided by the cell culture media; and thereby forming the programmable multicellular organoid and/or a 3D organ-specific tissue comprising the at least two divergent populations of differentiated or transdifferentiated programmable multicellular organoid and/or 3D organ-specific tissue cells. The at least one genetically-engineered inducible population of stem cells may comprise stem cells selected from the group consisting of pluripotent stem cells, multipotent stem cells, progenitor cells, terminally differentiated cells, endothelial cells, endothelial progenitor cells, immortalized cell lines, or primary cells. The at least one genetically-engineered inducible population of stem cells may be created by introducing a DNA delivery element comprising at least one of constitutive promoter, small molecule inducible promoter, cell-autonomous promoter, cell non-autonomous promoter, selection marker, or a combination thereof. The genetically-engineered inducible population of stem cells may overexpress at least one transcription factor selected from the group consisting of ETV2, NGNI, Tbr1, Fezf2, Ctip2, SATB2, LMXIA, NR4A2, Is11, Stl8, FOXA2, PITX3, Asc11, Smad7, Nr2fl, Dlx2, Dlx4, Nr2f2, Barhl2, and Lhxl. The differentiation and/or transdifferentiation of the of the at least one genetically-engineered inducible population of stem cells may be induced via the addition or removal of small molecules, growth factors, dissolved gases, or morphogens to or from the cell culture media. The differentiation and/or transdifferentiation of the at least one genetically-engineered inducible population of stem cells may be induced via the addition of doxycycline (DOX) into the cell culture media. In the method, the step of culturing may be in a differentiation medium. The differentiation medium may comprise doxycycline (DOX). The method my further comprise culturing a wild-type population of cells, and inducing differentiation of the wild-type population of cells into a different population of the programmable multicellular organoid and/or 3D organ-specific tissue cells. The differentiation of the wild-type population of cells may be induced via the addition or removal of small molecules, growth factors, dissolved gases, or morphogens to or from the cell culture media. In the method, the step of culturing may comprise culturing at least two genetically-engineered inducible populations of stem cells in the cell culture media.

Another embodiment relates to the use of the method descried anywhere herein to enable the tailoring of the initial ratio and/or composition of pluripotent cell populations to deterministically define the different cell types and quantity within the resulting 3D human tissue.

Yet another embodiment relates to a programmable multicellular organoid and/or a 3D organ-specific tissue produced by any method described herein.

Yet another embodiment relates to an in vitro method of generating functional human tissue construct, the method comprising: embedding a programmable multicellular organoid and/or a 3D organ-specific tissue of produced by any method escribed herein in a tissue construct, the tissue construct comprising a first vascular network and a second vascular network, each vascular network comprising one or more interconnected vascular channels; exposing the programmable multicellular organoid and/or a 3D organ-specific tissue to one or more biological agents, a biological agent gradient, a pressure, a pressure gradient, and/or an oxygen tension gradient, thereby inducing angiogenesis of capillary vessels to and/or from the programmable multicellular organoid and/or a 3D organ-specific tissue; and wherein the exposing step promotes vascularizing the programmable multicellular organoid and/or a 3D organ-specific tissue, the capillary vessels connecting the first vascular network to the second vascular network, thereby creating a functional human tissue construct comprising a single vascular network and a perfusable tissue structure. The programmable multicellular organoid and/or a 3D organ-specific tissue may be created by culturing a genetically-engineered inducible population of at least one of: pluripotent stem cells, multipotent stem cells, progenitor cells, terminally differentiated cells, endothelial cells, endothelial progenitor cells, immortalized cell lines, or primary cells in a cell differentiation media. The embryoid body or organoid may be created by culturing pluripotent or multipotent stem cells in a cell differentiation media. The one or more biological agents, the biological agent gradient, the pressure, the pressure gradient, and/or the oxygen tension gradient further direct development, differentiation, and/or functioning of the programmable multicellular organoid and/or a 3D organ-specific tissue. The programmable multicellular organoid and/or a 3D organ-specific tissue may be selected from the group consisting of: cerebral organoid or tissue, thyroid organoid or tissue, intestinal or gut organoid or tissue, hepatic organoid or tissue, pancreatic organoid or tissue, gastric organoid or tissue, kidney organoid or tissue, retinal organoid or tissue, cardiac organoid or tissue, bone organoid or tissue, and epithelial organoid or tissue. The programmable multicellular organoid and/or a 3D organ-specific tissue is exposed to the one or more biological agents and/or the biological agent gradient by at least one of: diffusion of one or more biological agents within the tissue construct; localized deposition of materials loaded with one or more biological agents within the tissue construct; localized de-novo production of growth factors by localized protein translation; or perfusion of one or both of the first and second vascular networks with one or more biological agents. The biological agents may comprise one or more of the following: growth factors, morphogens, small molecules, drugs, hormones, DNA, shRNA, siRNA, nanoparticles, mRNA, modified mRNA. The growth factors may comprise one or more of the following: vascular endothelial growth factor (VEGF), basic fibroblast growth factor (bFGF), sphingosine-I-phosphate (SIP), phorbol myristate acetate (PMA), hepatocyte growth factor (HGF), monocyte chemotactic protein-I (MCP-I), the angiopoietin ANG-I, the angiopoietin ANG-2, transforming growth factor beta (TGF-P), epidermal growth factor (EGF), human growth factor, matrix metalloproteinases (MMP’s), or histamine. In the method, the one or more interconnected vascular channels are formed by a manufacturing process or by a biological developmental process that may include at least one of vasculogenesis, angiogenesis, or tubulogenesis. The first vascular network and the second vascular network may be independently addressable. The first vascular network and the second vascular network may not be in contact with each other prior to the vascularizing step. The first vascular network may comprise an arterial plexus and the second vascular network may comprise a venous plexus. the single vascular network comprises at least one of an interpenetrating vascular network or a branched interpenetrating vascular network. The single vascular network may comprise interconnected arterial and venous channels. In the method, only one of the first and second vascular networks may be perfused with the one or more biological agents prior to the vascularizing step. Alternatively, both the first and second vascular networks may be perfused with the one or more biological agents, and wherein a biological agent concentration in the first vascular network may be different than a biological agent concentration in the second vascular network. In the method, both the first and second vascular networks may be perfused with the one or more biological agents, and wherein a biological agent concentration in the first vascular network may be the same as a biological agent concentration in the second vascular network. In the method, an oxygen partial pressure gradient may be introduced to one or both of the first and second vascular networks during perfusion. The embedding the programmable multicellular organoid and/or a 3D organ-specific tissue in the tissue construct may comprise: depositing one or more cell-laden filaments each comprising a plurality of viable cells on a substrate to form one or more tissue patterns, each of the tissue patterns comprising one or more predetermined cell types; depositing one or more sacrificial filaments on the substrate to form a vascular pattern interpenetrating the one or more tissue patterns, each of the sacrificial filaments comprising a fugitive ink; depositing the programmable multicellular organoid and/or a 3D organ-specific tissue within the vascular pattern; at least partially surrounding the one or more tissue patterns and the vascular pattern with an extracellular matrix composition; and removing the fugitive ink, thereby forming the tissue construct comprising the programmable multicellular organoid and/or a 3D organ-specific tissue embedded therein.

Yet another embodiment relates to an in vitro method of generating functional human tissue construct comprising: depositing one or more cell-laden filaments each comprising a bioink comprising at least one genetically-engineered inducible population of stem cells on a substrate or into a supporting matrix, to form one or more tissue patterns, each of the tissue patterns comprising at least one predetermined genetically-engineered inducible population of stem cells; depositing one or more sacrificial filaments on the substrate to form a vascular pattern; at least partially surrounding the one or more tissue patterns and the vascular pattern with an extracellular matrix composition; and removing the fugitive ink, thereby forming the functional tissue construct comprising tissue patterns comprising at least one predetermined genetically-engineered inducible population of stem cells embedded therein. The method may further comprise inducing direct differentiation and/or transdifferentiation of the at least one genetically-engineered inducible population of stem cells into at least two divergent populations of the programmable multicellular organoid and/or 3D organ-specific tissue cells. The differentiation and/or transdifferentiation of the at least one genetically-engineered inducible population of stem cells may be induced via the addition of doxycycline (DOX) into the cell culture media. The at least one genetically-engineered inducible population of stem cells may overexpress at least one transcription factor selected from the group consisting of ETV2, NGNI, Tbrl, Fezf2, Ctip2, SATB2, LMXIA, NR4A2, Isll, St18, FOXA2, PITX3, Ascll, Smad7, Nr2fl, Dlx2, Dlx4, Nr2f2, Barhl2, and Lhxl. The bioink may comprise at least two genetically-engineered inducible populations of stem cells. The at least one genetically-engineered inducible population of stem cells may be genomically programmed using an orthogonally induced differentiation platform. The bioink may comprise the at least one genetically-engineered inducible population of stem cells at cell density of at least 100 M cells/mL. The bioink may be composed of a cellular pellet comprising the at least one genetically-engineered inducible population of stem cells, formed via centrifugation of a cellular suspension and removal of the supernatant. The extracellular matrix components or rheological modifiers may optionally be added to the cellular suspension prior to centrifugation.

Yet further embodiment relates to a printhead head for simultaneously patterning one or more stem cells inks, wherein the stem cells can be orthogonally differentiated on demand post-printing.

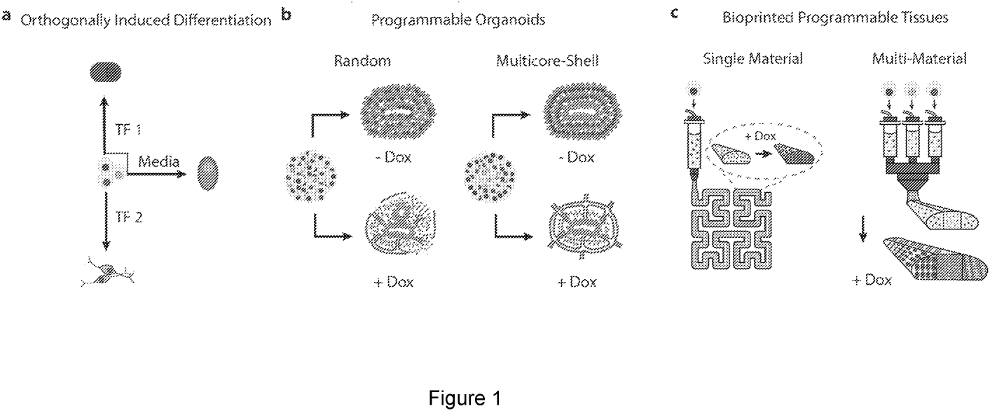

Yet further embodiment relates to a method as shown in FIGS. 1A, 1B and 1C.

### DETAILED DESCRIPTION

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, compositions, devices and materials are described herein.

As used herein and in the appended claims, the singular forms “a,” “and,” and “the” include plural referents unless the context clearly dictates otherwise. Thus, for example, reference to “a protein” includes a plurality of such proteins and reference to “the progenitor cell” includes reference to one or more progenitor cells known to those skilled in the art, and so forth.

PCT Pub. Nos. WO2015069619A1, WO2016141137A1, and U.S. Pat. Pub. No. US 2018/030409 are incorporated herein by reference in their entireties.

Described herein is a method of generating a programmable multicellular organoid and/or a 3D organ-specific tissue.

The term “organoid” refers to an embryoid body whose cells have undergone a degree of differentiation. The term “embryoid body” refers to a plurality of cells containing pluripotent or multipotent stem cells formed into a three dimensional sphere, spheroid, or other three dimensional shape. It is acknowledged that the distinction between an organoid and embryoid body remains undefined, and the use of the terms should be considered interchangeable.

The term “a programmable multicellular organoid” refers to an organoid that is formed from multiple populations of human induced pluripotent stem cells (hiPSCs) that upregulate differing sets of transcription factors, such that they undergo orthogonally induced differentiation from a pluripotent tissue into a multicellular differentiated tissue.

In the method, direct differentiation and/or transdifferentiation of the at least one genetically-engineered inducible population of stem cells into at least two divergent populations of the differentiated or transdifferentiated programmable multicellular organoid and/or 3D organ-specific tissue cells is induced concurrently, and independently of external cues, e.g., such as those provided by cell culture media. Simultaneous differentiation or transdifferentiation of genetically-engineered inducible population of stem cells (e.g., hiPSCs) into divergent cell types offers a pathway to achieving tailorable cellular complexity, patterned architecture, and function in engineered human organoids and tissues. The described method utilizes overexpression of various markers (e.g., overexpression of transcription factor(s) (TFs)) to drive the direct differentiation and/or transdifferentiation of the at least one genetically-engineered inducible population of stem cells into at least two divergent populations of the differentiated or transdifferentiated programmable multicellular organoid and/or 3D organ-specific tissue cells.

The terms “direct differentiation” or “directed differentiation” refer to the culture of pluripotent or multipotent stem cells in a condition that preferentially encourages the differentiation of the stem cell to a specific, more differentiated state. For example, a pluripotent stem cell may be cultured in a condition that results in an enriched population of specific multipotent stem cells such as neural progenitor cells. Alternatively, a multipotent stem cell such as a neural stem cell may be directly differentiated into a more differentiated state such as a neuron, astrocyte or oligodendrocyte.

The term “transdifferentiation” refers to the conversion of one cell type that may be a multipotent or unipotent stem cell, or a terminally differentiated mature cell phenotype to a different cell type that may be a different multipotent or unipotent stem cell, or a terminally differentiated mature cell phenotype. For example, a neural stem cell, a radial glia, or a neuron may be transdifferentiated into an endothelial cell.

To guide stem cell differentiation or transdifferentiation, one can either introduce extracellular cues by controlling the media composition or modulate the intracellular state by overexpressing various markers, such as transcription factors (TFs).

Many previous TF-based protocols synergistically apply external and internal cues to promote rapid and efficient cell differentiation to a single lineage; examples include the (I) derivation of endothelial cells cultured in endothelial cell growth medium, while overexpressing ETS translocation variant 2 (ETV2)^{22,23}, (2) derivation of neurons cultured in neuro-basal-A medium with B27, while overexpressing neurogenin-I and -2 (NGNI, NGN2)²⁴, and (3) derivation of hepatocytes cultured in hepatocyte medium, while overexpressing combinations of Gata4, Hnflα, Hnf4α, Foxa1, Foxa2, and Foxa3^{25,26}. Cakir et al. recently combined TF-driven differentiation with traditional cortical organoid culture by inducing overexpression of ETV2 in a subset of cells within cortical organoids, giving rise to a vascular endothelium akin to brain microvasculature²⁸.

While these approaches aim to differentiate cells into a singular phenotype, human tissues are composed of multiple cell types organized into hierarchically patterned structures. While the overexpression of one TF, such as Gata6, may create progenitors for multiple cell types²⁷, this strategy provides limited control over the precise composition and distribution of cell types in the resulting tissue because upregulation of Gata6 in iPSCs results in a variety of different cell types from a uniform population. Moreover, recent transcription factor overexpression protocols were designed to produce only one cell type of interest rather than the multitude of cell types and structural organization found in native human tissues.

Reported herein is an orthogonal differentiation platform/method, wherein, surprisingly and unexpectedly, genetically-engineered inducible population(s) of stem cells (e.g., pluripotent cells) are simultaneously co-differentiated into distinct cell types to generate organoids and bioprinted tissues with controlled composition and organization.

To demonstrate this platform, inventors orthogonally differentiated endothelial cells and neurons from genetically-engineered inducible population of stem cells, hiPSCs in a one-pot system containing either neural or endothelial stem cell-specifying media. By aggregating genetically-engineered inducible-TF and wild-type hiPSCs into pooled and multicore-shell embryoid bodies, vascularized and patterned cortical organoids were produced within days. Moreover, using multimaterial 3D bioprinting, 3D neural tissues that were orthogonally differentiated on demand were patterned into distinct layered regions composed of neural stem cells, endothelium, and neurons. Given the high proliferative capacity and patient-specificity of hiPSCs, the described platform/method provides a facile route for programming cells and multicellular tissues for drug screening and therapeutic applications.

The described method for generating programmable multicellular organoids and 3D organ-specific tissues can begin with a step of overexpressing a specific marker, such as a transcription factor in a pre-programmed cell population, which may be orthogonally differentiated to a specific cell type of interest with high efficiency, independent of media-driven differentiation. Multiple markers, such as multiple TFs, may be also overexpressed, each of which may also be orthogonally differentiated to a specific cell type of interest with high efficiency, independent of media-driven differentiation.

Exemplary markers include transcription factors, such as ETV2/ER71 for the differentiation of endothelial cells and NEUROGI for the differentiation of excitatory neurons.

Next, by pooling or printing populations of wild type (WT) and genetically-engineered inducible cell lines, multicellular organoids and 3D tissues can be can programmably generated on demand. The successful implementation of this approach/method requires the ability to simultaneously differentiate multiple cell lines, such as hiPSC lines in a one-pot protocol independent of external cues provided by the cell culture media.

Importantly, a method of inducing not only differentiation and/or transdifferentiation, but specific programmed cell behaviors by either adding a second gene that is induced by the same orthogonal signal/agent, or adding a second orthogonal induction cue, such as a different drug than doxycycline is described.

As such, in one embodiment, described herein is an orthogonally induced differentiation (OID) platform/method for achieving genomically programming human stem cells, organoids, and 3D bioprinted organ-specific tissues. In OID, e.g., doxycycline (DOX)-induced TF-overexpression in preprogrammed hiPSCs can override media-driven differentiation.

Although the described OID platform/method can be broadly applied, importantly, demonstrated herein is its utility by creating vascularized cortical organoids in pooled and multicore-shell motifs, as well as 3D cortical tissues composed of multiple cell types patterned in spatially distinct regions.

### Method (Overview)

In certain embodiments, the orthogonal differentiation platform/method can be used for rapidly programming and patterning human stem cells, organoids, and bioprinted organ-specific tissues within days. For example, as described in the examples below, overexpression of ETV2 and NGNI efficiently overrides the dual SMAD-inhibiting media cues, enabling the simultaneous generation of vascular endothelium and neurons in a one-pot system. Leveraging this capability, both vascularized and multicore-shell brain organoids were created from pooled and patterned embryoid bodies, respectively, which contained larger, more distinct ventricle-like structures. Through multimaterial 3D bioprinting of matrix-free wild-type and inducible-TF hiPSC inks, programmable, multicellular cortical tissues were generated in layered architectures that can be orthogonally differentiated on demand. With further advancement of stem cell differentiation protocols, it may soon be possible to efficiently and scalably produce the multitude of cells present in the human body. Looking ahead, the described OID platform/method offers a facile route for creating many types of programmable organoids and organ-specific tissues of interest for drug screening, disease modeling, and therapeutic applications.

### Method (Details)

In one embodiment described herein is a method of generating a programmable multicellular organoid, and/or a 3D organ-specific tissue.

The method includes:
(i) culturing at least one genetically-engineered inducible population of stem cells in a medium,
(ii) concurrently inducing direct differentiation and/or transdifferentiation of the at least one genetically-engineered inducible population of stem cells into at least two divergent populations of the differentiated or transdifferentiated programmable multicellular organoid and/or 3D organ-specific tissue cells; and
(iii) thereby forming the programmable multicellular organoid and/or a 3D organ-specific tissue comprising the at least two divergent populations of differentiated or transdifferentiated programmable multicellular organoid and/or 3D organ-specific tissue cells.

In certain embodiments, the described method begins with creating genetically-engineered inducible populations of cell. The term “genetically-engineered inducible population of stem cells” refers to transgenic stem cells that contain inducible promoters to upregulate the expression of a set of specific genes, such as transcription factors, upon addition of an inducing agent. Such inducing agents may be, e.g., chemical (including, e.g., doxycycline or cumate), temperature, or light based and will be described in more detail below. The genetically-engineered inducible population of stem cells may include pluripotent stem cells, multipotent stem cells, progenitor cells, terminally differentiated cells, endothelial cells, endothelial progenitor cells, immortalized cell lines, or primary cells.

The genetically-engineered inducible population of cells may be created by introducing a DNA delivery element comprising at least one of constitutive promoter, small molecule inducible promoter, cell-autonomous promoter, cell non-autonomous promoter, selection marker, or a combination thereof.

Examples of constitutive promoters include, e.g., EFI alpha, PGK, Ubiquitin, and CMV. Examples of small molecule inducible promoters include, e.g. doxycycline or cumate inducible promoters. Examples of cell-autonomous promoters include, e.g., cell type-specific promoters, such as DCX. Examples of cell non-autonomous promoter include, e.g., heat induced and light induced promoters.

Any suitable DNA delivery element known in the art may be used and can be selected from lentiviral inverted repeats, packaging signal (e.g., pLIX403 vector), transposon integration elements (e.g., PiggyBac vector), episomal replication elements. Alternatively, transient expression by electroporation or lipofection can be used.

In certain embodiments, orthogonal promoters can be used to upregulate the expression of a set of specific genes. The term “orthogonal promoters,” refers to two different promoter designs for which there are independent cues or orthogonal signals for gene induction. For example, if there are two inducible genes, ‘gene I’ activated by ‘promoter I’, and ‘gene 2’ activated by ‘promoter 2’, then promoter 1 and 2 are orthogonal if both of the following statements hold true:
1) There exists a ‘cue 1’ that specifically induces the expression of ‘gene 1’ without affecting the expression of ‘gene 2’; and
2) There exists a ‘cue 2’ that specifically induces the expression of ‘gene 2’ without affecting the expression of ‘gene 1’.

Selection markers may be selected from, e.g., drug resistance markers (e.g., puromycin, neomycin, and blasticidin). Alternatively, transient expression followed by dilution from cell division rather than selection markers may be used. The genetically-engineered inducible population of stem cells is created to overexpress a set of specific genes, such as transcription factors, upon addition of an inducing agent.

Examples of one or a set of specific genes include: transcription factors and PKC.

Examples of specific transcription factors that may be used to induce endothelial cells within any organoid (e.g., for vasculature) and to produce mixed populations within organoids include ETV2/ER71, FLII, ERG (Ginsberg et al. 2012 Cell), which induce differentiation of mature amniotic cells to endothelial cells; Gata2, FOXCI, FOXC2, HEYI, HEY2, SOX7, SOX18, PROX I (Park et al. 2013 Circulation Research), which induce differentiation of stem cells into various subtypes of endothelial cells (e.g. venous, arterial, lymphatic); Brachyury/T, which may be used for possible mesoderm induction, required for primitive streak formation in vivo.

Examples of specific transcription factors that may be used to induce neurons within any organoid (e.g., autonomic nervous system control of internal organs) include NEUROG ½ (Busskamp, et al., Molecular Systems Biology (2014)), which induce formation of excitatory neurons; ASCLI (Chanda, et al., Stem Cell Reports (2014)), which induce formation of excitatory neurons; ASCLI, BRN2, MYTIL, LHX3, HB9, ISLI, NGN2 (Son et al. 2011 Cell Stem Cell), which induce formation of motor neurons; and ASCLI, MYTIL, KLF7 (Wainger, et al., Nature Neuroscience (2014)), which induce formation of pain receptor neurons.

In certain embodiments, the genetically-engineered inducible population of stem cells overexpresses at least one transcription factor. The transcription factor may be selected from the group of ETV2, NGNI, Tbr1, Fezf2, Ctip2, SATB2, LMXIA, NR4A2, Isll, St18, FOXA2, PITX3, Ascll, Smad7, Nr2fl, Dlx2, Dlx4, Nr2f2, Barhl2, and Lhxl.

In certain further embodiments, the genetically-engineered inducible population of stem cells overexpresses at least two or more transcription factors selected from ETV2, NGN1, Tbr 1, Fezf2, Ctip2, SATB2, LMXl A, NR4A2, Isll, St18, FOXA2, PITX3, Ascll, Smad7, Nr2fl, Dlx2, Dlx4, Nr2f2, Barhl2, and Lhxl.

In certain embodiments, the method includes orthogonally inducing the expression of a constitutively-active PKC protein that dramatically enhances sprouting behavior of endothelial cells. PKC also encourages enhanced neurite outgrowth. It is important that the endogenous PKC signaling that directs neural outgrowth in cerebral organoids is not affected. Thus, by activating PKC in only the subset, sprouting in the endothelial cells is specifically achieved.

The term “PKC” is used as an acronym for protein kinase C, which may refer to any of the isoforms of protein kinase C, including PKC-alpha, PKC-betal, PKC-beta2, PKC-gamma, PKC-delta, PKC-epsilon, PKC-eta, PKC-theta, PKC-iota, and PKC-zeta.

By “sprouts,” or more specifically, “endothelial sprouts,” we are describing endothelial structures that have either undergone angiogenesis or vasculogenesis to generate tubular structures.

In certain embodiments, two or more genetically-engineered inducible populations of stem cells may be created in the same manner as described above.

In certain embodiments, two genetically-engineered inducible populations of stem cells may be used in the described method.

In one embodiment, the first population of stem cells may comprise, e.g., pluripotent stem cells, multipotent stem cells, progenitor cells, terminally differentiated cells, endothelial cells, endothelial progenitor cells, immortalized cell lines, or primary cells. The second population of stem cells may include neural progenitor cells. The neural progenitor cells can form at least one of excitatory neurons, inhibitory interneurons, motor neurons, dopaminergic neurons, pain receptor neurons, astrocytes, oligodendrocyte progenitor cells, oligodendrocytes.

Methods for culturing and differentiating stem cells into neuronal cells and tissues are known from Eiraku (2008), US 2011 10091869 A1 and WO 20111055855 A1, contents of which are incorporated by reference in their entirety. Methods described in U.S. Pat. Pub. No. US 2015/0330970 to Lancaster et al. and Lancaster et al., “Cerebral organoids model human brain development and microcephaly,” Nature 501, 373-379 (2013)), incorporated by reference herein.

Further culturing methods are known in the art. For example, culturing can take place on a low-adhesion substrate (Doetschman TC, et al., The in vitro development of blastocyst-derived embryonic stem cell lines: formation of visceral yolk sac, blood islands and myocardium. J Embryol Exp Morphol 87:27-45 (1985)), via a hanging drop method (Reubinoff BE, et al., Embryonic stem cell lines from human blastocysts: somatic differentiation in vitro. Nat Biotechnol 18(4):399-404 (2002)), via aggregation in microwells (Mohr JC, et al., 3-D microwell culture of human embryonic stem cells. Biomaterials 27(36):6032-42 (2006), via aggregation in microchannels (Onoe H, et al., Differentiation Induction of Mouse Neural Stem Cells in Hydrogel Tubular Microenvironments with Controlled Tube Dimensions. Adv Healthc Mater. (2016), doi: 10.1002/adhm.201500903), or by using a spinning bioreactor (Carpenedo RL, et al., Rotary suspension culture enhances the efficiency, yield, and homogeneity of embryoid body differentiation. Stem Cells 25(9):2224-34 (2007)).

The step of culturing of the described method includes culturing at least one genetically-engineered inducible population of stem cells in a medium. The step of culturing may be in a differentiation medium.

In certain embodiments, the differentiation medium may be a neural differentiation media (exemplary neural differentiation medium is described below). In other embodiment, the differentiation media may be endothelial cell-specifying media. Other differentiation media may also be used. Examples include cardiomyocyte-specifying media, proximal tubule epithelium-specifying media, and mesoendoderm-specifying media.

In certain embodiments, the differentiation medium may include an inducing agent to induce the differentiation and/or transdifferentiation of genetically-engineered inducible population(s) of stem cells. Exemplary inducing agents include, e.g., chemical (including, e.g., DOX or cumate), temperature, or may be light-based. In one embodiment, the differentiation medium includes DOX. In alternative embodiments, the differentiation medium may include a drug different than DOX or a different inducing agent, such as tetracycline, cumate, mRNA, siRNA, shRNA, DNA, temperature, or light.

The inducing step includes concurrently inducing, in “one-pot” system, direct differentiation and/or transdifferentiation of the at least one genetically-engineered inducible population of stem cells into at least two divergent populations of the differentiated or transdifferentiated programmable multicellular organoid and/or 3D organ-specific tissue cells. The term “concurrently” in the context of this application refers to the fact that multiple populations of hiPSCs are undergoing differentiation concurrently to form the differentiated cells or tissue. Importantly, the concept of inducing not only differentiation, but specific programmed cell behaviors by either adding a second gene that is induced by the same orthogonal signal/agent, or adding a second orthogonal induction cue like a different drug than doxycycline is described.

In one embodiment, the inducing step may include concurrently inducing direct differentiation and/or transdifferentiation of the at least one genetically-engineered inducible population of stem cells that express transcription factors.

The term “divergent” in the context of “divergent populations of differentiated or transdifferentiated programmable multicellular organoid and/or 3D organ-specific tissue cells” refers to the fact that the cells formed via orthogonally induced differentiation are from distinct lineages, or even distinct germ layers. As such, the process of concurrent orthogonally induced differentiation can drive cells to differentiate in a divergent fashion towards multiple distinct cell types, instead of towards a single cell type.

The step of inducing direct differentiation and/or transdifferentiation of the genetically-engineered inducible population of cells can comprise introducing at least one cue selected from the group consisting of transcription factors, drugs, small molecules, growth factors, morphogens, hormones, DNA, shRNA, siRNA, nanoparticles, mRNA, modified mRNA, heat, light, dissolved gases, and mechanical stimulation.

In certain other embodiments, the differentiation and/or transdifferentiation of the of the at least one genetically-engineered inducible population of stem cells can be induced by removal of at least one cue selected from the group consisting transcription factors, drugs, small molecules, growth factors, morphogens, hormones, DNA, shRNA, siRNA, nanoparticles, mRNA, modified mRNA, heat, light, dissolved gases from the media.

In certain specific embodiments, doxycycline may be added to the cell culture media to induce differentiation and/or transdifferentiation of the genetically-engineered inducible population of cells.

In certain embodiments, the direct differentiation may be accompanied by a secondary induction of a different gene, e.g., a second orthogonal induction. This secondary induction may occur at an earlier time, simultaneously, or at a later time than the first gene induction. The secondary gene induction may be via providing at least one cue selected from the group consisting of transcription factors, drugs, small molecules, growth factors, morphogens, hormones, DNA, shRNA, siRNA, nanoparticles, mRNA, modified mRNA, heat, light, and mechanical stimulation.

In certain embodiments, the cue selected for the secondary gene induction is the same as the cue selected for the step of inducing direct differentiation and/or transdifferentiation of the genetically-engineered inducible population of cells. Alternatively, the cue selected for the secondary gene induction is different, and orthogonal, from the cue selected for the step of inducing direct differentiation and/or transdifferentiation of the genetically-engineered inducible population of cells.

In certain embodiments, the first population of the embryoid body or organoid cells can undergo a further development due to induction of a secondary gene. The induction of the secondary gene includes induction of expression of, e.g., a constitutively-active PKC protein thereby enhancing at least one of a sprouting behavior of the first population of the embryoid body or organoid cells, or neurite outgrowth. The first population of the embryoid body or organoid cells may be endothelial cells.

The ratio of the first population the embryoid body or organoid cells to the second population of the embryoid body or organoid cells may be 1:1, 1:2, 1:3. 1:4, 1:5, etc. or 5:1. 4:1. 3:1, 2:1.

As a result of the described method, embryoid bodies or organoids comprising at least two different or divergent populations of organoid or embryoid body cells (i.e., a first population of embryoid body or organoid cells and a second population of the embryoid body or organoid cells) can be produced and later vascularized. For example, in certain embodiments, the embryoid body or organoid can comprise multiple populations of cells (i.e., at least two different cell lineages), such as endothelial and neuronal, obtained by differentiation of iPSCs using the same culture condition.

In certain further embodiments, the method may include culturing a wild-type population of cells and inducing differentiation of the wild-type population of cells into a different population of the programmable multicellular organoid and/or 3D organ-specific tissue cells.

Similarly, the differentiation of the wild-type population of cells may be induced via the addition or removal of small molecules, growth factors, dissolved gases, or morphogens from the cell culture media.

In one embodiment, specific cell types can be produced by the described method. As described herein, surprisingly and unexpectedly, forced overexpression of intracellular TFs can operate independently of media-driven differentiation to generate specific cell types, each with very high (near 100%) efficiency (FIG. 1a).

Specifically, the embodiment shown in FIG. 1a relates to a method that includes culturing two genetically-engineered inducible populations of stem cells and a wild type population of cells in a medium. The first genetically-engineered inducible population of stem cells overexpresses “TFI” and the second genetically-engineered inducible population of stem cells overexpresses “TF2.” These two populations of genetically-engineered stem cells are pooled together with a wild type population in a 2D culture; such on a well plate, petri dish, or a transwell membrane; or in a 3D culturing environment, such as in a printed tissue or organoid. Direct differentiation and/or transdifferentiation of the first and the second genetically-engineered inducible populations of stem cells is concurrently induced due to overexpression of the specific TFs by the addition of an inducing agent. As a result, the genetically-engineered inducible populations of stem cells are directly differentiated and/or transdifferentiated into two divergent populations of the differentiated or transdifferentiated tissue cells, e.g., endothelial and neuronal cells. The wild type population of cells is directly differentiated and/or transdifferentiated into a third type of tissue cells.

In another embodiment, multicellular and spatially patterned organoids can be produced by the described method. When applied to randomly pooled (left drawings of FIG. 1(b)) or multicore-shell (right drawings of FIG. 1(b)) embryoid bodies, orthogonally induced differentiation can be used to construct multicellular randomly and spatially patterned organoids (FIG. 1b).

The term “spatially patterned organoid” refers to organoids in which multiple genetically-engineered inducible populations of stem cells and/or wild type populations of stem cells are each restricted to a specific region of the organoid, such as the core of the organoid, the inner shell of the organoid, or outer shell of the organoid. Spatially patterned organoids can be organized into alternative regions, such as anterior or posterior regions, dorsal and ventral regions.

The term “randomly patterned organoid” refers to organoids in which multiple genetically-engineered inducible populations of stem cells and/or wild type populations of stem cells are evenly distributed throughout the organoid.

Specifically, in the embodiment shown in FIG. 1b, DOX-induced TF-overexpression in preprogrammed hiPSCs can override media-driven differentiation. Specifically, two genetically-engineered inducible populations of stem cells and a wild type population of cells are cultured in a cell medium. The first genetically-engineered inducible population of stem cells overexpresses “TFI” and the second genetically-engineered inducible population of stem cells overexpresses “TF2.” Direct differentiation or transdifferentiation of the first and the second genetically-engineered inducible populations of stem cells is concurrently induced by introducing distinct cues, such as DOX, which is independent of the culture media.

Two genetically-engineered inducible populations of stem cells and a wild type population of stem cells are simultaneously aggregated together to form a randomly patterned embryoid body. Randomly patterned embryoid bodies formed by this method are cultured in media containing distinct cues, such as DOX, and become randomly patterned and multicellular organoids.

In another embodiment, two genetically-engineered inducible populations of stem cells and a wild type population of stem cells are sequentially aggregated by first aggregating a wild type population of stem cells, followed several hours later by a second aggregation of a genetically-engineered inducible population of stem cells, followed several hours later by a third aggregation of a different genetically-engineered inducible population of stem cells. Following the third aggregation, spatially patterned embryoid bodies are formed. Spatially patterned embryoid bodies are cultured in media containing distinct cues, such as DOX, and become spatially patterned and multicellular organoids.

In yet another embodiment, multicellular tissue constructs that mimic native tissue architectures can be produced by the described method. Specifically, when the described OID approach/method is coupled with 3D bioprinting of densely cellular, matrix-free WT and inducible-TF hiPSCs inks, pluripotent tissues can be patterned and subsequently transformed in situ to multicellular tissue constructs that mimic native tissue architectures. An inducible-TF hiPSC ink made from genetically-engineered inducible populations of stem cells can be used to print a pluripotent, that upon the addition of an inducing agent, differentiates into a divergent population of tissue cells, such as endothelial cells or neuronal cells. Two inducible-TF hiPSC inks, made from two genetically-engineered populations of stem cells, can be coupled with a hiPSC ink made from wild type stem cells by using multimaterial 3D bioprinting to print pluripotent tissues, that upon the addition of an inducing agent, differentiates into a multicellular tissue (FIG. 1c).

Specifically, in one embodiment, described herein is an in vitro method of generating functional human tissue construct that includes embedding a programmable multicellular organoid and/or a 3D organ-specific tissue created by the method described herein and as illustrated, e.g., in FIG. 1(b)) in a tissue construct. In one example, the programmable multicellular organoid and/or a 3D organ-specific tissue may be created by culturing a genetically-engineered inducible population of at least one of: pluripotent stem cells, multipotent stem cells, progenitor cells, terminally differentiated cells, endothelial cells, endothelial progenitor cells, immortalized cell lines, or primary cells.

The tissue construct can includes a first vascular network and a second vascular network, where each vascular network comprising one or more interconnected vascular channels. The method further includes exposing the programmable multicellular organoid and/or a 3D organ-specific tissue to one or more biological agents, a biological agent gradient, a pressure, a pressure gradient, and/or an oxygen tension gradient, thereby inducing angiogenesis of capillary vessels to and/or from the programmable multicellular organoid and/or a 3D organ-specific tissue. The programmable multicellular organoid and/or a 3D organ-specific tissue can then be vascularized, where the capillary vessels connecting the first vascular network to the second vascular network, thereby creating a single vascular network and a perfusable tissue structure.

The step of embedding the programmable multicellular organoid and/or a 3D organ-specific tissue in the tissue construct may include depositing one or more cell-laden filaments each comprising a plurality of viable cells on a substrate to form one or more tissue patterns, each of the tissue patterns comprising one or more predetermined cell types; depositing one or more sacrificial filaments on the substrate to form a vascular pattern interpenetrating the one or more tissue patterns, each of the sacrificial filaments comprising a fugitive ink; depositing the programmable multicellular organoid and/or a 3D organ-specific tissue within the vascular pattern; at least partially surrounding the one or more tissue patterns and the vascular pattern with an extracellular matrix composition; and removing the fugitive ink, thereby forming the tissue construct comprising the programmable multicellular organoid and/or a 3D organ-specific tissue embedded therein.

The method also includes exposing the programmable multicellular organoid and/or a 3D organ-specific tissue to one or more biological agents, a biological agent gradient, a pressure, a pressure gradient, and/or an oxygen tension gradient, thereby inducing angiogenesis of capillary vessels to and/or from the programmable multicellular organoid and/or a 3D organ-specific tissue. Importantly, the exposing step promotes vascularizing the programmable multicellular organoid and/or a 3D organ-specific tissue, the capillary vessels connecting the first vascular network to the second vascular network, thereby creating a functional human tissue construct comprising a single vascular network and a perfusable tissue structure.

The programmable multicellular organoid and/or a 3D organ-specific tissue may be exposed to the one or more biological agents and/or the biological agent gradient by at least one of: diffusion of one or more biological agents within the tissue construct; localized deposition of materials loaded with one or more biological agents within the tissue construct; localized de-novo production of growth factors by localized protein translation; or perfusion of one or both of the first and second vascular networks with one or more biological agents.

The biological agents may be growth factors, morphogens, small molecules, drugs, hormones, DNA, shRNA, siRNA, nanoparticles, mRNA, and modified mRNA.

The growth factors include vascular endothelial growth factor (VEGF), basic fibroblast growth factor (bFGF), sphingosine-1-phosphate (S1P), phorbol myristate acetate (PMA), hepatocyte growth factor (HGF), monocyte chemotactic protein-I (MCP-I), the angiopoietin ANG-I, the angiopoietin ANG-2, transforming growth factor beta (TGF-P), epidermal growth factor (EGF), human growth factor, matrix metalloproteinases (MMP’s), or histamine.

The one or more biological agents, the biological agent gradient, the pressure, the pressure gradient, and/or the oxygen tension gradient can further direct development, differentiation, and/or functioning of the programmable multicellular organoid and/or a 3D organ-specific tissue.

The programmable multicellular organoid and/or a 3D organ-specific tissue may be cerebral organoid or tissue, thyroid organoid or tissue, intestinal or gut organoid or tissue, hepatic organoid or tissue, pancreatic organoid or tissue, gastric organoid or tissue, kidney organoid or tissue, retinal organoid or tissue, cardiac organoid or tissue, bone organoid or tissue, and epithelial organoid or tissue.

In certain embodiments, the one or more interconnected vascular channels are formed by a manufacturing process or by a biological developmental process that may include at least one of vasculogenesis, angiogenesis, or tubulogenesis.

The first vascular network and the second vascular network may be independently addressable.

Alternatively, the first vascular network and the second vascular network may not be in contact with each other prior to the vascularizing step described above.

In certain embodiments, the first vascular network can comprise an arterial plexus and the second vascular network can comprise a venous plexus.

In certain alternative embodiments, the single vascular network can comprises at least one of an interpenetrating vascular network or a branched interpenetrating vascular network.

In certain further embodiments, the single vascular network can comprise interconnected arterial and venous channels.

In certain embodiments, only one of the first and second vascular networks may be perfused with the one or more biological agents prior to the vascularizing step described above.

In certain alternative embodiments, both the first and second vascular networks may be perfused with the one or more biological agents, where a biological agent concentration in the first vascular network is different than a biological agent concentration in the second vascular network. Alternatively, both the first and second vascular networks are perfused with the one or more biological agents, where a biological agent concentration in the first vascular network is the same as a biological agent concentration in the second vascular network.

In certain embodiment, an oxygen partial pressure gradient may be introduced to one or both of the first and second vascular networks during perfusion.

Certain further embodiments relate to an in vitro method of generating functional human tissue construct that includes depositing one or more cell-laden filaments each comprising a bioink comprising at least one genetically-engineered inducible population of stem cells on a substrate or into a supporting matrix, to form one or more tissue patterns, each of the tissue patterns comprising at least one predetermined genetically-engineered inducible population of stem cells; depositing one or more sacrificial filaments on the substrate to form a vascular pattern; at least partially surrounding the one or more tissue patterns and the vascular pattern with an extracellular matrix composition; and removing the fugitive ink, thereby forming the functional tissue construct comprising tissue patterns comprising at least one predetermined genetically-engineered inducible population of stem cells embedded therein.

The method may further include a step of inducing direct differentiation and/or transdifferentiation of the at least one genetically-engineered inducible population of stem cells into at least two divergent populations of the programmable multicellular organoid and/or 3D organ-specific tissue cells. The differentiation and/or transdifferentiation of the at least one genetically-engineered inducible population of stem cells may be induced via the addition of doxycycline (DOX) into the cell culture media.

The genetically-engineered inducible population of stem cells may be genomically programmed using an orthogonally induced differentiation platform.

The at least one genetically-engineered inducible population of stem cells may overexpress at least one transcription factor. The transcription factor may be, e.g., ETV2, NGNI, TbrI, Fezf2, Ctip2, SATB2, LMXIA, NR4A2, Isll, St18, FOXA2, PITX3, Ascll, Smad7, Nr2fl, Dlx2, Dlx4, Nr2f2, Barhl2, and Lhxl.

In the described method, in certain embodiments, the bioink can comprise the at least one genetically-engineered inducible population of stem cells at cell density of at least 100 M cells/mL. In alternative embodiments, the bioink can include at least two genetically-engineered inducible populations of stem cells. In certain embodiments, the bioink may be composed of a cellular pellet that includes at least one genetically-engineered inducible population of stem cells, formed via centrifugation of a cellular suspension and removal of the supernatant.

The genetically-engineered inducible population(s) of stem cells used in the method may be genomically programmed using an orthogonally induced differentiation platform described herein.

In certain embodiments, extracellular matrix components or rheological modifiers may be optionally added to the cellular suspension prior to centrifugation.

Certain further embodiments relate to a programmable multicellular organoid and/or a 3D organ-specific tissue produced by any method described herein.

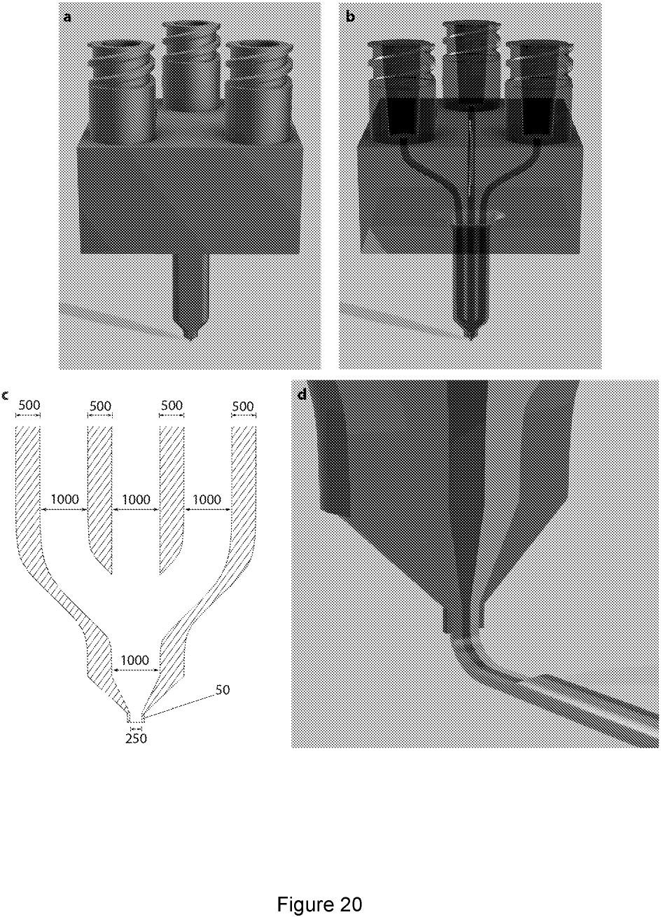

Certain further embodiments relate to a printhead head for simultaneously patterning one or more stem cells inks, wherein the stem cells can be orthogonally differentiated on demand post-printing. The printhead is illustrated in FIG. 20.

### EXAMPLES

### Example 1

### Methods Summary for Vascularizing Organoids

Human induced pluripotent stem cells (iPSC’s) are maintained in culture on vitronectin coated non-tissue culture treated 6-well plates in mTeSRI medium. To passage, cells are treated with accutase for 5 minutes, rinsed with DMEM/FI2 containing 15 mM HEPES, and gently triturated by pipetting up and down twice with a P1000 pipette tip. Cells are then replated in mTeSRI medium containing 10 µM Rho-kinase inhibitor (ROCK-i). After 12-20 hours, the cells are replaced in mTeSRI without ROCK-i, and media is changed every day.

To form embryoid bodies, at ‘Day 0’, cells are passaged as described above, except for using a longer, 15 minute, accutase treatment to separate the colonies into single cells to aid accurate counting. Once counted, the iPSC’s are added to an aggrewell 400 plate, at a total density of 500 cells per micro-well, centrifuged at 100 g for 3 minutes, and cultured in 2 ml accutase containing 10 µM ROCK-i to form embryoid bodies. At this point, if a genetically modified population of iPSC’s is to be used, they can be added and mixed to the single cell suspension of wild-type iPSC’s at a defined cell-count ratio, added to the aggrewell plate, then centrifuged at 100 g for 3 minutes to generate a mixed population of cells in each micro well. After 20 hours, ‘Day 1’, embryoid bodies are formed in the microwells, and the media is replaced with aggrewell medium without ROCK-i. Media is changed daily until ‘Day 3’, at which point embryoid bodies can be removed from the microwells by gentle pipetting and cleaned by rinsing on the surface of a 40 µm reversible cell filter, before flipping the filter to release the embryoid bodies using 5 ml of neural induction medium, comprising DMEM/F 12 medium supplemented with 1:100 N2 supplement, 1:100 Glutamax and 1:100 MEM-Non-essential amino acids (MEM-NEAA) and 10 µg/ml heparin. Embryoid bodies were transferred to ultra-low adhesion 6-well plates for suspension culture, and are agitated twice per day to prevent aggregation.

After four days in suspension culture ‘Day 7’, cells are implanted into a microvascular scaffold formed by either pin-casting or molding around a sacrificial printed filament of either Pluronic F-127 or gelatin. The matrix is comprised of matrigel mixed with either rat rail collagen type 1 (2 mg/ml) or fibrinogen (10 mg/ml). The vascular network contains two independent networks, an ‘arterial’ and a ‘venous’ network. The culture medium is switched to neural differentiation medium, phase 1, comprising a 1:1 mixture of DMEM/F12 and Neurobasal, supplemented with 1:200 N2 supplement, 1:100 B27 supplement without vitamin A, 1:200 MEM-NEAA, IX Glutamax, and IX P-mercaptoethanol. Next, specific angiogenic factors are added to one or both networks to encourage vascular sprouting. The terms “sprouting,” “sprouts,” or more specifically, “endothelial sprouts” mean endothelial structures that have either undergone angiogenesis or vasculogenesis to generate tubular structures.

Media may be pumped through the two independent networks by means of a peristaltic pump, and media may be replaced every two days.

At ‘Day 11’, the media may be replaced with neural differentiation medium, phase 2, which is the same as neural differentiation medium, phase 1, except that the B27 without vitamin A is replaced with B27 with vitamin A.

An orthogonal signal, such as dox can be added at, e.g., 100 ng/ml to the medium at any phase of the differentiation process to induce the transdifferentiation or directed differentiation of the genetically modified iPSC’s. Furthermore, a second, orthogonal signal, such as cumate, mRNA, or other inducing agents, can be added to the media conditions described to induce sprouting of induced endothelium to enhance the formation of a capillary plexus that connects both venous and arterial systems. Adding an angiogenic factor can provide a gradient to induce directional angiogenesis.

Once angiogenesis has occurred sufficiently to connect the two networks, the positive pressure can be applied, via a peristaltic pump to only the arterial side, allowing fluid flow through the connecting capillaries to the venous side.

### Example 2

### Methods

Cell culture. PGPI hiPSCs were utilized for this study (Coriell, #GM23338). Cells were verified for pluripotency by flow-cytometry and cultured between passages 20 and 50. PGPIs were cultured and passaged without antibiotics in mTeSRI (STEMCELL Technologies, #05850) on tissue culture plates coated with hESC-qualified Matrigel (Corning, #354277) during inducible cell line generation or with growth-factor reduced Matrigel (Corning #354230) for growing hiPSCs prior to differentiation, organoid formation, or bioprinting. For passaging, hiPSCs were washed with phosphate buffered saline (PBS) without calcium and magnesium, dissociated using TrypLE Express (Life Technologies, #12604013), and then seeded at 300k cells/well in a 6-well plate supplemented with 10 µM Y-27632 (Selleck Chemicals, #S1049) for I d, and subsequently maintained in mTeSRI with daily media changes. For cryo-storage, cells were dissociated with TrypLE Express, counted, and resuspended in mFreSR (STEMCELL Technologies, #05854) at a concentration of 10⁶ cells/ml (STEMCELL Technologies, #5854) using a CoolCell LX freezing block (Biocision, #BCS-405) overnight at -80° C., then stored in liquid nitrogen for long-term storage.

TF-induced clonal hiPSC cell lines. A Gateway compatible, doxycycline-inducible (Tet-On), PiggyBac vector with a puromycin resistance gene was provided by the Church Lab. pDONR221-NEUROG1 (HsCD00040492) from the DNASU^{48,49} repository was cloned into a barcoded version of PBAN001 using a Gateway reaction with Gateway LR Clonase 11 Enzyme mix (Invitrogen, #12538200) with a 20bp barcode: 5′ CAAAGTGAAAC-CAGAGTCGC 3′ (SEQ ID NO:31). The construct was delivered into PGP1 cells via Super PiggyBac Transposase Expression vector (SB1, #PB210PA-1) by nucleofection at a mass ratio of 1:4 (PBAN-NGN1):(SPB) in 2 µl of total reaction volume, utilizing the Lonza 4D Nu-cleofector X-Unit (Lonza, #AAF-1002X) with a 20 µl P3 solution kit (Lonza, #V4XP-3032) with 600k WT cells in each 20 µl reaction well as per manufacturer’s instructions. After nucleofection, cells in solution were plated on a 6-well tissue culture-treated plate coated with Matrigel in mTeSRI supplemented with 10 uM Y-27632 for one day. Selection by addition of 1 µg/ml puromycin (Gibco, #A1113803) occurred after cells reached 70% confluency and supplemented with 5 µM Y-27632 for one day. Cells were washed once using PBS without calcium or magnesium (Gibco, #14190250) and maintained on mTeSRI supplemented with 10 µM Y-27632 until colonies were visible.

Confluent polyclonal iNeuron colonies were then dissociated with TrypLE for 7 min, centrifuged at 300 g for 5 min, and resuspended in 500 µl of mTeSRI supplemented with 10 µM Y-27632 and penicillin-streptomycin (MilliporeSigma, #P4333), strained through a 35 µm cell-strainer (Falcon, #352235) and immediately placed on ice. We used the SH-800 s Sony Flow Sorter (100 µm chip nozzle) to sort single cells into Matrigel-coated 96-well plates with each well containing 150 µl of mTeSRI supplemented with CloneR (STEMCELL Technologies, #05888). Following the manufacturer’s instructions, we maintained the cells until confluent and seeded candidate colonies into two 48-well plate wells, one for validation through induction via addition of 500 ng/ml doxycycline hyclate (MilliporeSigma, #D989 1), and another well for further expansion/passaging. iEndo cells, a clonal PGP1 ETV2 isoform 2 line, was constructed in a similar fashion to the iNeurons using the TFome transcription factor library²⁹.

To engineer fluorescently labelled hiPSC lines iEndo-mKate2 and eGFP-WT hiPSCs, the FUGW plasmid (Addgene # 14883), which encodes constitutively expressed GFP, was modified to express membrane-bound mKate2 (FUmemKW). These two plasmids were packaged into lentiviruses as described previously²⁹, and FUGW and FUmemKW lentiviruses were transduced into WT PGP1 and iEndo hiPSCs to generate green and red cell lines respectively. hiPSCs were sorted on a FACSAria to obtain a pure population of cells that express the fluorescent proteins uniformly.

2D orthogonal induced differentiation assays. To study OD in neural-specifying conditions, 300k WT PGP1, iEndo PGP1, or iNeuron PGP1 cells were seeded in each well of Matrigel coated 6 well plates in neural induction media (NIM), consisting of DMEM/F12 with GlutaMAX (Gibco, #10565018) supplemented with 1:100 N2 supplement (Gibco, #17502048), 1:100 minimal essential media non-essential amino acids (MEM-NEAA), 1 µg/ml heparin (Sigma, H3149), 5 µM SB431542 (BioGems, #3014193), and 100 nM LDN193189 (BioGems, #1066208), with or without 500 ng/mL doxycycline hyclate. NIM was further supplemented with 10 µM Y-27632 at the time of seeding. 24 h after plating, media was changed to NIM with or without doxycycline. Media was changed every 48 h for 6 days of culture.

To study OD in endothelial specifying conditions, WT or iNeuron PGP1 cells were grown to 60-70% confluency in mTeSRI, at which point cells were rinsed once in PBS and the media was changed to RPM1 1640 (Gibco, #11875093) supplemented with 1:50 B27 minus insulin (Gibco, #A1895601) and 6 µM CH1R99021 (BioGems, #2520691), with or without 500 ng/mL doxycycline, for 2 days. On day 2, the media was changed to RPM1 1640 with 1:50 B27 minus insulin and 3 µM CHIR99021. On day 4, the media was changed to EGM-2 (PromoCell, #C-22111) with 10 µM SB431542, 50 ng/mL vascular endothelial growth factor (VEGF) (PeproTech, #100-20), and 25 ng/mL fibroblast growth factor 2 (PeproTech, # 100- 18B), with or without 500 ng/mL doxycycline. The media was changed again on day 6 and cells were kept in culture until day 8.

Fluorescent time-lapse imaging during stem cell differentiation. 500 µl of cold Matrigel was added to a single well in a 6-well tissue-culture treated plate and allowed to gel by warming to 37° C. for 30 min. 150k WT-eGFP PGP1 cells and 150k iEndo-mKate2 PGP1 cells were seeded in mTeSRI supplemented with Y-27632 and 500 ng/ml doxycycline and cultured for 1 d. On day 2 of culture, the mTeSRI medium was removed and the plate was gently rinsed with PBS before replacing with NIM with 500 ng/ml doxycycline hyclate added. The plate was transferred to an inverted fluorescence microscope (Zeiss Axio Observer Z1) with an environmental chamber (CO₂ and temperature controlled) and imaged through a 10x objective onto a Photometrics Evolve electron-multiplying charge-coupled device (EMCCD), tiling over a 4×4 grid every 30 min. Media was changed daily and images were captured over a 4 d period. Tiled images were stitched automatically in Zeiss’ Zen Blue software with a 5% tile-overlap and processed using ImageJ.

### Flow Cytometry

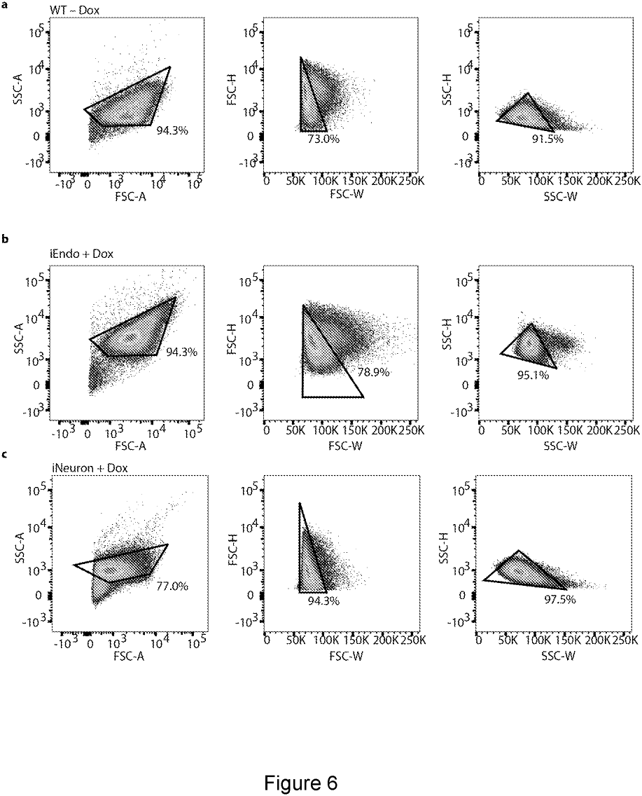

For intracellular and surface marker staining, cells were dissociated using Trypsin-EDTA, fixed for 15 min in BD Cytofix (BD, #554714) and washed 3× in PBS with 3% wt/vol bovine serum albumin (BSA). Samples were then aliquoted in a 96-well at 5× 10⁵ cells/well, permeabilized in BD Perm/Wash (BD, #554723) solution for 15 min, pelleted via centrifugation at 300 g, and the supernatant was aspirated. Samples were incubated for 45 min in antibodies diluted in BD Perm/Wash at a concentration of 10⁷ cells/ml per dilution in the dark and washed three times in BD Perm/Wash before flow cytometry experiment was performed. Color compensation was performed on each run utilizing BD Anti-Mouse IG (BD, #552843) and fluorescently conjugated antibodies. All flow cytometry measurements were performed on the BD LSR Fortessa cell analyzer. Flow files (.fcs) were processed in FlowJo 10.6.1 (gating strategy included in FIG. 6). Plots were generated with Prism 8.4.0. Fluorescently conjugated antibodies utilized in flow cytometry assays are listed in Table 1.

TABLE 1

Fluorescently conjugated antibodies for flow cytometry

| Name | Fluoro-phore | Vendor | Clone | Catalog Number | Dilution |
|---|---|---|---|---|---|
| OCT¾ | AF647 | BD Biosciences | 40/Oct-3 | 560329 | 1:20 |
| PAX6 | PE | BD Biosciences | O18-1330 | 561552 | 1:20 |
| PAX6 | AF488 | BD Biosciences | O18-1330 | 561664 | 1:20 |
| VE-Cadherin | BV421 | BD Biosciences | 55-7H1 | 565671 | 1:20 |
| MAP2 | PE | MilliporeSigma | AP20 | FCMAB318PE | 1:100 |

HUVEC-hiPSC aggregate cohesion assay. HUVEC-RFP cells (Angio-Proteomie, #cAP-0001RFP), WT-eGFP PGP1 cells, and iEndo-mKate2 PGP1 cells were dissociated from 70%-80% confluency by incubating cells in Gentle Cell Dissociation Reagent (STEMCELL Technologies, #07174) for 12 min at 37° C., then resuspended in DMEM/F12 with HEPES (Gibco, #11330032). Cells were then centrifuged at 250 g for 5 min before resuspending and single-cell filtering in either EB culture media (EBCM) for iEndo-mKate2 cells and WT-eGFP cells, consisting of mTeSRI supplemented with 4 mg/ml polyvinyl alcohol (PVA, MilliporeSigma, #P8136), or a 1:1 mix of EBCM and EGM-2 for 1:1 cocultures of HUVEC-RFPs:WT-eGFPs. The PVA stock solution was prepared by fully dissolving PVA in stirred deionized water at 90° C. to a stock concentration of 200 mg/ml. Wells of a 24-well AggreWell™ 400 plate (STEMCELL Technologies, #34411) were seeded with either 1:1 iEndo-mKate2:WT-eGFP cells in EBCM with 10 µM Y-27632, or a 1:1 HUVEC-RFP cells:WT-eGFP cells in a 1:1 mix of EBCM and EGM-2, with 10 µM Y-27632. 24 h after plating, media in both conditions was changed to EBCM without Y-27632. 48 h after plating, aggregates were transferred to suspension culture in non-adherent T25 flasks in EBCM on an orbital shaker rotating at 53 RPM. Aggregates were imaged 24 h after plating and 48 h after plating on a Zeiss LSM710 confocal microscope.

Vascularized cortical organoid culture. Cortical organoid culture was adapted from previously established protocols^{13,50,51}. EBs were formed on day -3 by dissociating hiPSC monolayers at 70-80% confluency with EDTA dissociation reagent (EDR) for 12 min at 37° C., 5% CO₂, which consists of 0.5 mM EDTA in PBS, supplemented with an additional 0.03 M NaCl. Cells were then resuspended in DMEM/F12 with HEPEs and centrifuged at 250 g for 5 min. Wells of a 24-well AggreWell™ 800 plate (STEMCELL Technologies, #34815) were treated with an anti-adhesive coating of 0.2% Pluronic F127 in PBS and seeded with 1.5× 10⁶ cells/well using either WT PGP1 hiPSCs or a 1:2 ratio of iEndo:WT PGP1 hiPSCs in EBCM supplemented with 10 µM Y-27632. The day after aggregation (day -2), EBCM media was replaced with fresh EBCM to remove Y-27632 within the AggreWell™ 800 plates. On day -1, EBs were transferred into suspension culture in fresh EBCM in non-adherent T25 flasks on an orbital shaker rotating at 53 rpm.

Neural differentiation began on day 0 (after 1 d of suspension culture). EBs were transferred to neural induction media (NIM) with doxycycline. After 3 d in NIM, organoids were transferred into 80 µl cold collagen/Matrigel gel droplets, formed on dimpled parafilm, which was prepared similarly to a previously established protocol⁴³. Final collagen and Matrigel concentrations were 4 mg/ml rat tail collagen type 1 (Corning, #354249) and 25% Matrigel (Corning). Gel droplets containing organoids were gelled at 37° C. for 15 min before being transferred back into NIM in ultra-low adherence 6-well plates (Corning, #3471), which were held stationary. 3 d after implanting organoids into gels (day 6 after the start of neural induction), organoids were transferred onto an orbital shaker rotating at 90 RPM and the media was changed to neural differentiation media 1 (NDM 1), consisting of a 1:1 mix of DMEM/F12 with GlutaMAX and Neurobasal media (Gibco, #21103049) supplemented with 1:200 GlutaMAX (Gibco, #35050061), 1:200 MEM-NEAA, 1:200 N2 supplement, 1:100 B27 supplement without vitamin A (Gibco, #12587010), 1:4000 insulin (MilliporeSigma, #19278), 10 ng/ml VEGF, 20 ng/ml EGF (PeproTech, #AF-100-15), 20 ng/ml FGF2, 50 µM β-mercaptoethanol (MilliporeSigma, #M6250), and 500 ng/ml doxycycline. 4 d later, half the media was replaced with fresh NDM 1. On day 13, after the start of neural induction, a full media changed was performed to replace all media with neural differentiation media 2 (NDM2), consisting of a 1:1 mix of DMEM/F12 with GlutaMAX and Neurobasal media supplemented with 1:200 GlutaMAX, 1:200 MEM-NEAA, 1:200 N2 supplement, 1:100 B27 supplement (Gibco, #17504044), 1:4000 insulin, 10 ng/ml VEGF, 20 ng/ml EGF, 20 ng/ml FGF2, 50 µM β-mercaptoethanol, and 500 ng/ml doxycycline. Half media changes of NDM2 were performed every 4 d until 25 d after neural induction. 25 d after the start of neural induction, a full media change was performed to replace all media with NDM3, consisting of a 1:1 mix of DMEM/F12 with GlutaMAX and Neurobasal media supplemented with 1:200 GlutaMAX, 1:200 MEM-NEAA, 1:200 N2 supplement, 1:100 B27 supplement with vitamin A (Gibco, #12587010), 1:4000 insulin, 10 ng/ml VEGF, 20 ng/ml BDNF (PeproTech, #450-02), 50 µM β-mercaptoethanol, and 500 ng/ml doxycycline. Half media changes of NDM3 were performed as needed, every 1-4 days, for the duration of organoid culture. Vascularized cortical organoids were cultured for up to 45 d.

Multicore-shell cortical organoid culture. Wild type PGP1-hiPSCs were dissociated using EDR and seeded into anti-adhesive treated V-bottom 96-well plates (Globe Scientific, #120130) at a density of 1,000 cells/96-well well in EBCM + 10 µM Y-27632 for 12 h to generate organoid cores. After 12 h, iNeurons were dissociated using EDR and added to each EB at an additional 2,000 cells/96-well well and were allowed to aggregate for 12 h in EBCM with 10 µM Y-27632. Then, iEndo cells were dissociated using EDR and an additional 2,000 cells were added to each EB and allowed to aggregate over 12 h in EBCM + 10 µM Y-27632. 12 h after the final aggregation, media was fully replaced with EBCM. Neural differentiation was initiated 48 h after the start of aggregation by changing the media to NIM within the 96-well plate. 24 h after initiating neural differentiation, organoids were harvested from the 96-well plate and implanted into 80 µl collagen/Matrigel gel droplets. Multicore-shell organoids were then cultured using the vascularized cortical organoid protocol.

### Cryo-Sectioning and Immunostaining

Organoids were fixed in 4% paraformaldehyde for 30 min and rinsed 3× in PBS. For cryosections, fixed organoids were incubated for 2 d at 4° C. in PBS containing 30% wt/vol sucrose and then transferred into a 1:1 solution of Optimal Cutting Temperature compound (OCT) (Tissue-Tek, #4583) and PBS containing 30% wt/vol sucrose for 90 min. Next, the tissue was placed into a cryostat tissue mold, which was subsequently filled with 100% OCT solution and frozen at -20° C. on a cryostat Peltier cooler. The tissue was sectioned using 40- to 60-µm slices and transferred onto a Superfrost Plus glass slide (VWR Inc., #48311-703). Sections were stored at -20° C. before immunostaining.

For immunostaining of cryosections and whole cortical organoids, tissues were permeabilized for 30 min in PBS containing 0.1% Triton-X, then blocked for > 1 h in PBS containing 2% donkey serum. Next, tissue sections were incubated overnight and whole-mount organoids were incubated for 1-3 d in primary antibodies in PBS containing 2% donkey serum. Tissues were rinsed 3× in PBS containing 0.05% Tween-20 (PBST), then incubated in secondary antibodies in PBS with 2% donkey serum for an equal amount of time as primary antibodies. Cell nuclei were labeled with 300 nM of 4′,6-diamidino-2- phenylindole (DAP1) in PBS for 10 min, followed by 3 rinses in PBST. Tissue sections and whole mount organoids were imaged on a Zeiss LSM710 confocal microscope. All primary and secondary antibodies used in immunostaining are listed in Table 2.

TABLE 2

Primary immunostaining antibodies

| Name | Host Animal | Vendor | Catalog Number | Dilution |
|---|---|---|---|---|
| CD31 | Mouse | Abcam | ab9498 | 1:200 |
| Nestin | Mouse | Abcam | ab6320 | 1:200 |
| vWF | Mouse | Abcam | ab194405 | 1:200 |
| CT1P2 | Rat | Abcam | ab18465 | 1:200 |
| ZO1 | Rabbit | Abcam | ab96587 | 1:100 |
| NRPI | Rabbit | Abcam | ab25998 | 1:250 |
| PAX6 | Rabbit | BioLegend | PRB-278P | 1:200 |
| N-Cadherin | Mouse | Cell Signaling Technologies | 14215 | 1:200 |
| VE-Cadherin | Rabbit | Cell Signaling Technologies | 2500S | 1:200 |
| NeuN | Rabbit | Cell Signaling Technologies | 24307 | 1:200 |
| TBR1 | Rabbit | Cell Signaling Technologies | 49661S | 1:200 |
| tRFP | Rabbit | Evrogen | AB233 | 1:200 |
| MAP2 | Mouse | MilliporeSigma | MAB3418 | 1:200 |
| SOX2 | Goat | R&D Systems | AF2018 | 1:200 |
| β-111-Tubulin | Mouse | R&D Systems | MAB1195 | 1:400 |
| OCT¾ | Rabbit | Santa Cruz Biotechnology | sc-5279 | 1:200 |
| Ulex-Rhodamine | N/A | Vector Laboratories | RL- 1062 | 1:200 |

iDISCO+ tissue clearing. Cortical organoids were cleared and immuno-labeled using an adapted version of the iDISCO+ protocol⁵². Briefly, the organoids were dehydrated using a methanol/water gradient over the course of 6 h, then delipidated using a 67% dichloromethane (DCM)/33% methanol solution for 3 h before bleaching in 5% hydrogen peroxide in methanol overnight. Next, they were rehydrated in a reverse methanol/water gradient over the course of 6 h and transferred into PBS. They were then immuno-labeled using the same immunolabeling protocol described above and dehydrated a second time using a methanol water gradient over 6 h before further delipidation in 67% DCM/33% methanol for 3 h. The organoids were then rinsed twice in 100% DCM before rehydration over 6 h with a reverse methanol/water gradient. Finally, they were index matched using Easylndex (LifeCanvas Technologies) and imaged using a Zeiss LSM710 confocal microscope. 3D renderings of cleared cortical organoids were made using the 3Dscript ImageJ plugin⁵³.

Angiotool analysis of vascularized cortical organoids. Confocal z-stacks were taken of iDISCO cleared WT only and WT + iEndo organoids. Individual optical sections at z = 10.8, 119, 227, 335, and 442 µm within a single organoid were used for Angiotool analysis⁵⁴. Optical sections were preprocessed by raising the LUT threshold to 20 to eliminate background noise. Angiotool analysis was conducted using a vessel diameter of 5 and a small particles filter of 40.

RNA extraction and RT-qPCR of cortical organoids. Cortical organoids were cultured using protocols described above. RNA extraction was performed utilizing the Ambion PureLink RNA mini kit (Invitrogen, #12183020). Homogenization of single organoids suspended in Matrigel-collagen hydrogel droplet was performed utilizing a handheld homogenizer (Bel-art, #F65000-0000) with accompanying RNAse-free, DNAse-free single-use pestles (Bel-art, #F65000-0006) with organoids suspended in 600 µl of lysis buffer, provided in the Ambion PureLink Kit. On-column DNAse 1 (Invitrogen, #AM2222) digestion was also performed during the RNA-extraction process. Extracted RNA samples were validated utilizing an Agilent 2200 TapeStation for RNA integrity number (RIN) score quantification with all samples utilized in this study validated to be above 8.8. cDNA synthesis was performed using the SuperScript IV First-Strand Synthesis System (Invitrogen, #18091050) with Oligo d(T) primer and with the input cDNA for each sample normalized to 10 ng/µl for a total of 110 ng/reaction. cDNA was validated with included kit controls by gel-electrophoresis. RT-qPCR experiments were performed with IDT’s PrimeTime Gene Expression Primer/Probes (ZEN/FAM), utilizing IDT’s PrimeTime Gene Expression Master Mix (IDT, #1055771). Curves were obtained on a BioRad cf96 qPCR machine. Scripts for data processing of C_{q} values were created in Python and graphs were made utilizing Prism 8.4.0. A list of all primers and probes used is provided in Table 3.

TABLE 3

Primers and probes for RT-qPCR

| Gene | 5′ Primer | SEQ ID NO: | 3′ Primer | SEQ ID NO: |
|---|---|---|---|---|
| ACTB | ACAGAGCCTCGCCTTTG | 1 | CCTTGCACATGCCGGAG | 2 |
| B2M | GGACTGGTCTTTC-TATCTCTTGT | 3 | ACCTCCATGATGCTGCTTAC | 4 |
| CDHS | GAACCAGATGCACATT-GATGAAG | 5 | TGCCCACATATTCTCCTTT-GAG | 6 |
| PAX6 | ATTTCAGAGCCCCATATTC GAG | 7 | GACACCACCGAGCTGATTC | 8 |
| PECAM I | AACAGTGTTGACATGAA-GAGCC | 9 | TGTAAAACAGCAC-GTCATCCTT | 10 |
| POLR2A | GAGCAATCGGCCTGTCAT | 11 | ACATCAGGAGGTTCATCAC-TTC | 12 |
| POU5FI | GTT-GGAGGGAAGGTGAAGTTC | 13 | TGTGTC-TATCTACTGTGTCCCA | 14 |
| RBFOX3 | CGGTCGTGTATCAG-GATGGA | 15 | GCCGTAACTGTCGCTGTAG | 16 |
| SOX2 | GTACAACTCCATGAC-CAGCTC | 17 | CTTGACCACCGAACCCAT | 18 |
| TUBB3 | GTTCTGGGAAGTCATCAG-TGA | 19 | CTCGAGGCACGTACTTGTG | 20 |

| | | |
|---|---|---|
| Gene | Probe | SEQ ID NO: |
| ACTB | /56-FAM/TCATCCATG/ZEN/GTGAGCTGGCGG/31ABkFQ/ | 21 |
| B2M | /56-FAM/CCTGCCGTG/ZEN/TGAACCATGTGACT/31ABkFQ/ | 22 |
| CDH5 | /56-FAM/TGAGTCGCA/ZEN/AGAATGCCAAGTACCT/31ABkFQ/ | 23 |
| PAX6 | /56-FAM/CAGCATGCA/ZEN/GAACAGTCACAGCG/31ABkFQ/ | 24 |
| PECAMI | /56-FAM/AAATGGGAA/ZEN/GAACCTGACCCTGCA/31ABkFQ/ | 25 |
| POLR2A | /56-FAM/ACTGCTGTG/ZEN/AGTGTGTCCTGCA/31ABkFQ/ | 26 |
| POU5FI | /56-FAM/TGCTCTTGA/ZEN/TTTTAATCCCACATCATGTATCACT/31AB-kFQ/ | 27 |
| RBFOX3 | /56-FAM/ATGGTGCTG/ZEN/AGATTTATGGAGGCTACG/31ABkFQ/ | 28 |
| SOX2 | /56-FAM/CACCTACAG/ZEN/CATGTCCTACTCGCA/31ABkFQ/ | 29 |
| TUBB3 | /56-FAM/ATCCGCTCC/ZEN/AGCTGCAAGTCC/31ABkFQ/ | 30 |

Single Cell Sequencing of Day 25 Organoids. WT iPSChiPSC, Mixed, and Multicore-shell cortical organoids were cultured using the previously described methods until day 25. Dissociation was performed using a previously described protocol¹⁴. Cell encapsulation (GEM generation) was performed using the Chromium Next GEM Chip G Single Cell Kit (10x Genomics, PN-1000127) and accompanying Chromium Next GEM Single Cell 3′ GEM, Library & Gel Bead Kit v3.0 (10× Genomics, PN-1000128) on the 10× Chromium Controller (10x Genomics, PN-1000202), with a targeted recovery of 5000 single cells per sample. Post GEM-RT Cleanup & cDNA Amplification was performed using the recommended protocols. cDNA QC was performed on the Qiagen Qubit 3.0 platform using the Qubit dsDNA HS Assay Kit (Thermo Fischer Scientific, Q32854). Quantification was performed using the Agilent High Sensitivity DNA Analysis Kit (Agilent, 5067-4626) on the Agilent 2100 Bioanalyzer Instrument (Agilent, G2939BA). 3′ Gene Expression Library Construction was performed, and samples were indexed using the Single Index Kit T Set A (10x Genomics, PN-1000213). Indexed samples were further validated for quality using Qubit and Bioanalyzer Kits and normalization of concentration was performed using the manual’s suggested methodology prior to pooling for submission for sequencing. The pooled library was QC’ed using qPCR and ran on a single lane of a NovaSeq S4 flow cell with v 1.5 Reagent kits and 2x150bp, with a targeted depth of 50,000 reads per cell. Raw reads were processed using 10× Genomics’ CellRanger 5.0 Software Suite against the GRCh38 human reference genome.

In Seurat, cells with more than 3000 UM1s, 1300 genes, and less than 20% counts from mitochondria genes were used for further processing. Counts were normalized using SCTransform within each batch, followed by integration of two batches using canonical correlation analysis (CCA) and mutual nearest neighbors (MNNs) in Seurat. Cluster identification was performed using the R Package Seurat 4.0 ‘FindClusters’, UMAP analysis was performed by the ‘RunUMAP’ function, and UMAP plotting was performed by running the ‘Featureplot’ function. Differential expression analysis was performed based on the Wilcoxon rank-sum test and the ‘Find Markers’ function. The P values were adjusted for multiple testing using the Bonferroni correction. Gene Ontology analysis was performed using the clusterProfiler 3.8.1 R package using the ‘enrichGO’ function and plotted using its ‘dotplot’ function. Clusters were annotated using the SingleCellNet R package with the training reference dataset being primary human brain samples GSE132672. The classifier was trained against the ‘subtype’ class detailed in their annotation metadata with the cells implied in subtype ‘outlier’ removed for clarity.

Automated Annotations using SingleCellNet. Cluster generation were annotated using the singleCellNet⁴⁰ R package with the training reference dataset being primary human brain samples GSE132672. The classifier was trained against the ‘subtype’ class detailed in their metadata with the cells implied in subtype ‘outlier’ removed for clarity. The classifier was utilized to suggest identities for clusters in 0.1 resolution.

Quantification of organoid patterning. Organoids were mounted in EasyIndex Solution and imaged on a Zeiss LSM710 confocal microscope. A custom MATLAB script was written to analyze CellTracker™ labeled organoids. Each pixel was assigned to one of the three colors used in the CellTracker™ study based on the fluorescence intensity and pictures were binarized using a maximum entropy thresholding function. Hell et al⁵⁵ give the following formula to account for the focus shift that results from imaging into high refractive index media:

$$A F P = \frac{{n}_{2}}{{n}_{1}} N F P$$

where AFP is the actual focus position (z position of the voxel), NFP is the nominal focus position (imaged z position of the voxel), and n₁ and n₂ are the refractive indexes of air (n = 1) and Easy Index (n = 1.47), respectively. Easylndex is assumed to have minimal dispersion across visible wavelengths of light. The center position of each organoid is identified, and the distance is calculated between each voxel and the center position for all voxels. Distances were normalized to the radius of each organoid and were summed up to a histogram plot normalized to histogram surface area.

To measure the length of apical neuroepithelium in ventricles, organoid sections were stained with N-cadherin and Sox2 and imaged on a Zeiss LSM710 confocal microscope. N-cadherin positive regions surrounded with radially organized Sox2+ cells were considered to be ventricles and traced in ImageJ.

3D bioprinting. A triple-material co-flow nozzle (printhead) was designed using Fusion360 (Autodesk Inc.), and exported as a stereolithography (.stl) file. The nozzles were printed using a stereolithography 3D printer (Perfactory Aureus, EnvisionTEC), using HTM 140v2 resin (EnvisionTEC) with a layer height of 50 µm and a calibrated power of 700 mW. Printed nozzles were first rinsed and their internal channels were flushed using isopropyl alcohol. They were then dried under a stream of air and further cured under ultraviolet (UV) illumination using an Omnicure lamp (EXFO).

Densely cellular bioinks were created from one 90% confluent T225 flask of hiPSCs, which were loaded a syringe prior to printing. These hiPSCs were first rinsed in PBS without calcium or magnesium and incubated in TrypLE for 7 min at 37° C., 5% CO₂ and then lifted off and added into 37° C. DMEM/F12 with HEPES and centrifuged at 250 g for 5 min. After the supernatant was removed, the cells were resuspended in DMEM/F12 with HEPES, and the suspension was filtered through a 40 µm cell strainer (BD Falcon, #352340) to generate a single cell suspension, which was subsequently pelleted via centrifugation at 250 g for 5 min. The supernatant was aspirated, the remaining cell pellet was resuspended in 250 µl of mTeSRI containing 10 µM Y-27632, transferred to a 1.6 ml Eppendorf tube, and front loaded into a 1 ml syringe (Covidien Kendall, #8881901014). The 1 ml syringe was centrifuged, with the tip facing upwards, at 750 g for 5 min to form a cell pellet that rests upon the syringe plunger. The supernatant was aspirated inside the syringe by inserting a 1.5 inch-long aspiration nozzle via the tip of the syringe, leaving the pellet intact. Next, the pellet was brought to the tip of the 1 ml syringe by gently manipulating the syringe plunger. The tip of the syringe was then pressed against the back-end of a 250 µl gas-tight glass syringe (Hamilton #81 120) without its plunger in place, and the pellet was back-loaded into the glass syringe by gently pressing the 1 ml syringe plunger. Next, the gas-tight plunger was inserted into the glass syringe to bring the rear-loaded pellet up to the tip of the syringe, taking care to avoid introducing air at the plunger-pellet interface. The custom-built printhead was attached to the syringe, which was then mounted on a six-axis motion control stage⁹ fitted with a custom-built syringe pump¹⁰, for 3D bioprinting.

For bioprinting single-cell filaments, tapered milled metal nozzles with an inner diameter of 50 µm (GPD Global, #10/4794) or 100 µm (GPD Global, #10/4793) were attached to the tip of a glass syringe loaded with the cell pellet. For printing multicellular filaments, a 3D printed triple nozzle was connected to three loaded syringes which were mounted to a single syringe pump that simultaneously drives extrusion of all three inks. Ink extrusion was controlled using an Arduino microcontroller and custom-built stepper motor driver. Printer motion was controlled through using manually written G-code. G-code for the cortical tissue architecture was created from an image taken of a GWl l human brain section⁵⁶. PGPI hiPSC filaments were printed onto ThinCert transparent 0.4 µm pore sized transwells in a 6-well plate (Greiner Bio-One, #657641). Immediately after printing, the two parts of the gelatin-fibrin pre-gel solution were mixed at a 4:1 ratio and cast overtop the printed filament(s). A combined total of 500 µl of mixed gelatin-fibrin gel was used to encapsulate the printed tissue. 1 ml of EBCM supplemented with 10 µM Y-27632 and 1 U/ml thrombin (MilliporeSigma, #T4648) was added beneath the transwell to keep the cells hydrated. Prints were first incubated for 10 min at room temperature to cross-link the fibrin gel, then transferred in an incubator at 37° C., 5% CO2 for 30 min. The media was then removed and replaced with 4 ml/well EBCM supplemented with 10 µM Y-27632, 100 U/ml penicillin-streptomycin, 11.5 KiU/ml aprotinin and 500 ng/ml doxycycline. The next day, media was changed to NIM supplemented with 100 U/ml penicillin-streptomycin and 11.5 KiU/ml aprotinin. Prints were maintained at 37° C., 5% CO2 and a full media change was performed every other day until day 6.

After printing, the densely cellular filamentary features were encapsulated in a gelatin-fibrin gel, which was prepared using an modified version of a prior protocol⁴³. Briefly, a 15 wt/vol% gelatin solution was produced by adding gelatin powder (MilliporeSigma, #G2500) to PBS without calcium or magnesium and stirring for 12 h at 70° C. and adjusting the pH to 7.5 using 1 M NaOH. Part 1 of the gel solution was made by diluting the 15 wt/vol% gelatin 1:1 with mTeSRl and adding 2.5 mM CaCl₂, 10 µM Y-27632 and 1 U/mL thrombin for a final 7.5 wt/vol% gelatin mix. Part 2 of the gel solution was produced by dissolving lyophilized bovine blood plasma fibrinogen (MilliporeSigma, #34l576) at 37° C. in sterile PBS at 50 mg/ml. Both parts of the pre-gel solution were maintained in separate tubes at 37° C. before use.

The density of hiPSCs in bioinks was estimated by dispensing 100 µl of bioink from the glass syringe, resuspending the cells in 4 ml of mTeSRI, and counting the number of cells using a cell counter. The density of Oct4+ cells was calculated via multiplying the total number of cells/ml in the bioink by the percentage of cells that were Oct4+, as measured by flow cytometry, using cells obtained from bioink samples.

LIVE/DEAD quantification of printed filaments. Prior to printing, a solution containing LIVE/DEAD Viability/Cytotoxicity kit (Invitrogen, L3224) reagents was prepared in EBCM containing 20 µM Y-27632, 4 µM calcein-AM, and 8 µM ethidium homodimer-1 (EthD-1). Then, single-cell filaments were printed as described above. After printing, the LIVE/DEAD solution was used to resuspend the printed cellular filament into a single cell filament. The cell suspension was then mixed 1:1 with 15 wt/vol% gelatin for a final gel concentration of 7.5 wt/vol% gelatin, 10 µM Y-27632, 2 µM calcein-AM, and 4 µM EthD-l. The gel was then set at RT and the cells were imaged on a Zeiss LSM700 confocal microscope. Image processing was done in lmageJ to count the number of cells staining for calcein-AM and EthD-l.

CellTracker labeling of hiPSCs. For CellTracker studies of aggregated organoids and printed tissue filaments, CellTracker fluorescent dye was reconstituted in DMSO, then diluted to the working concentration in mTeSRI. The hiPSCs were then incubated with dye for 30 min at 37° C. 2.5 µM of CellTracker™ Green 5-chloromethylfluorescein diacetate CMFDA (Molecular Probes, #C7025) was used to label WT-PGPI cells, 2.5 µM of CellTracker™ Orange 5-(and-6)-(((4-chloromethyl)benzoyl)amino)tetramethyl-rhodamine CMTMR (Molecular Probes, C2927) was used to label iNeuron cells, and 250 nM of CellTracker™ Deep Red (Molecular Probes, #C34565) was used to label iEndo cells. In triple prints, WT-PGPl were labeled with 5 µM CellTracker™ Blue 7-amino-4-chloromethyl-coumarin CMAC (Molecular Probes, #C2l 10), iNeuron-PGPI was labeled with 2.5 µM CellTracker™ Green CMFDA (Molecular Probes, #C7025), iEndo-PGPl was labeled with 2.5 µM CellTracker™ Red (Chloromethyl 6-(4(5)-amino-2-carboxyphenyl)-1,2,2,4,8,10,10,11-octamethyl-1,2,10,11-tetrahydrodipyrido(3,2-b: 2,3-i] xanthylium) CMTPX (Molecular Probes, #C34552). Cells were then washed with PBS without calcium or magnesium and dissociated for printing or organoid aggregation. Organoids were fixed 24 h after the final aggregation and bioprinted tissues were fixed 4 h after printing in 4% paraformaldehyde for 30 min.

Measurement of printed filament width. Single-cell filaments were stained with LIVE/DEAD reagents and multicellular filaments were stained with CellTracker Blue CMAC, Green CMFDA, and Red CMTPX and imaged under a Zeiss LSM700 confocal microscope. The total signal intensity from all channels was then converted to grayscale in lmageJ and averaged parallel to the filament to create longitudinal average intensity profile. Filament widths were calculated as the FWHM of the averaged intensity profile.

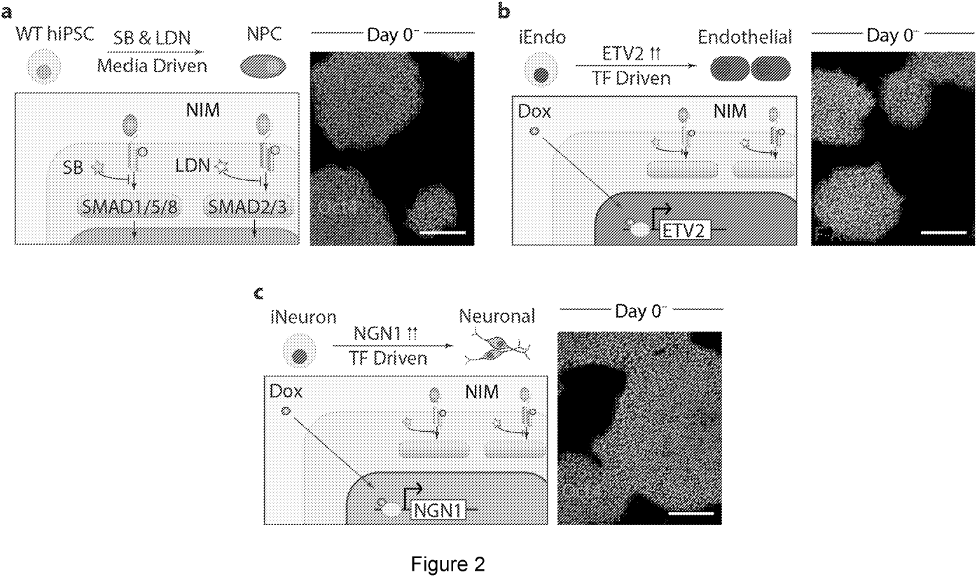

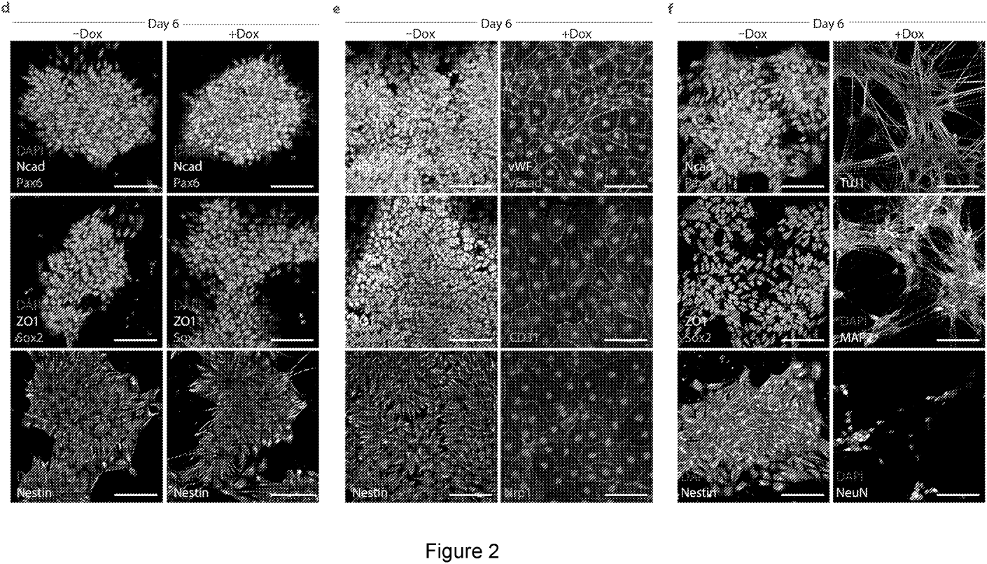

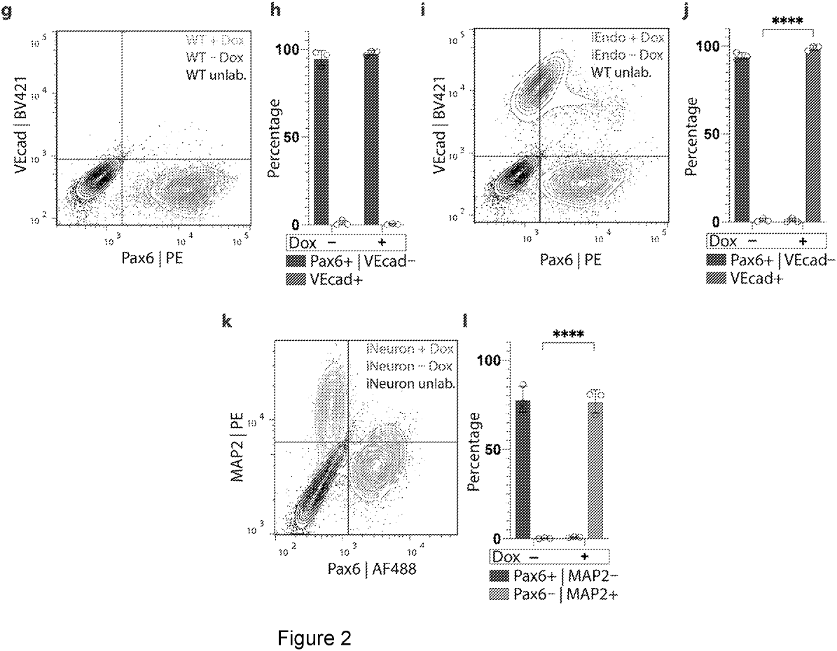

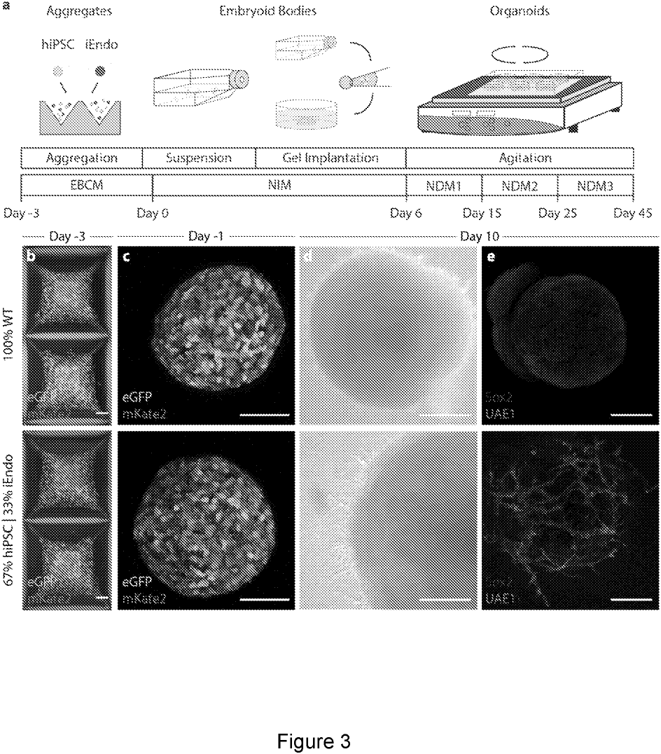

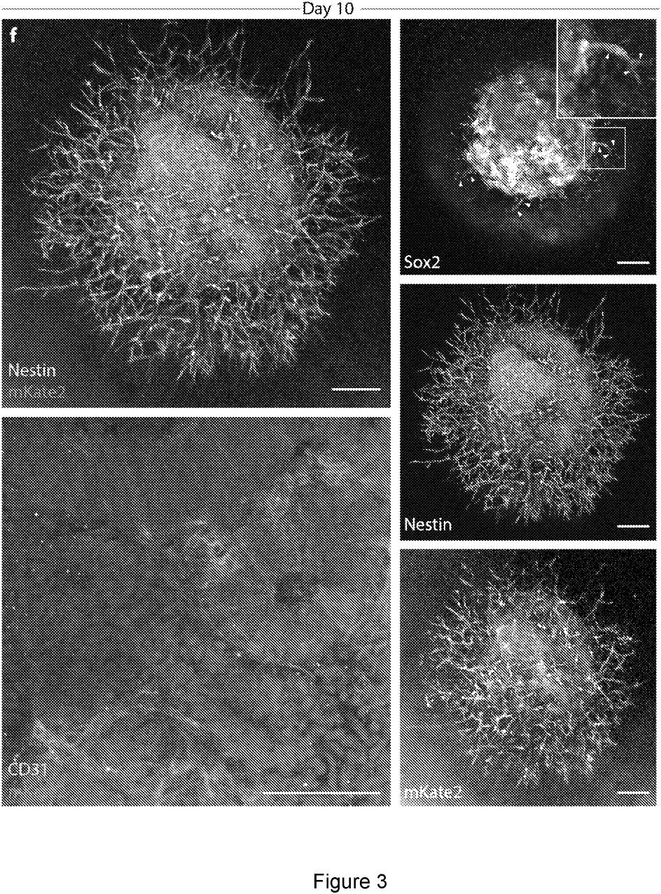

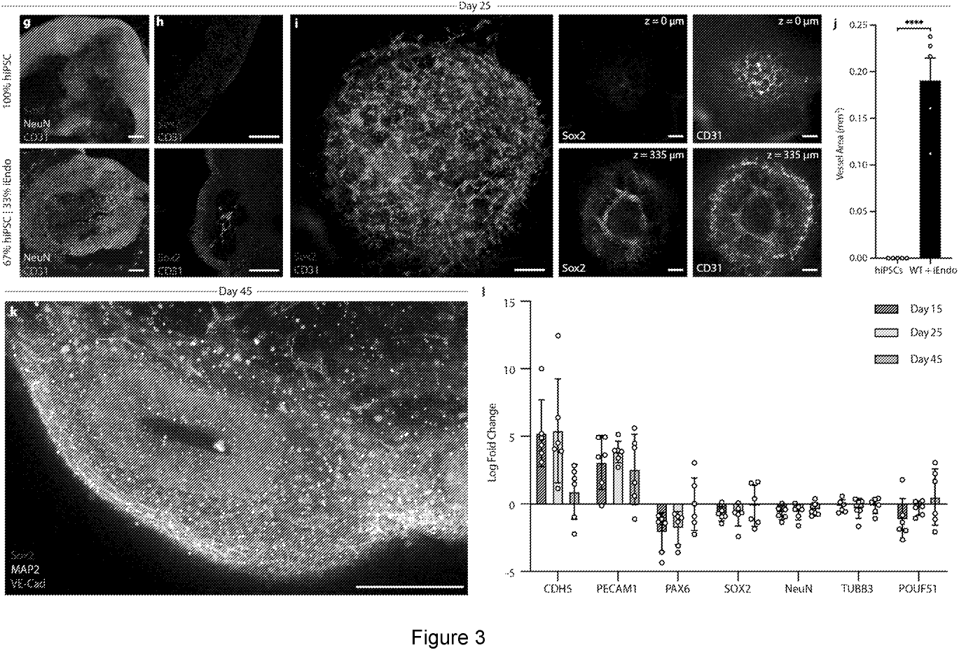

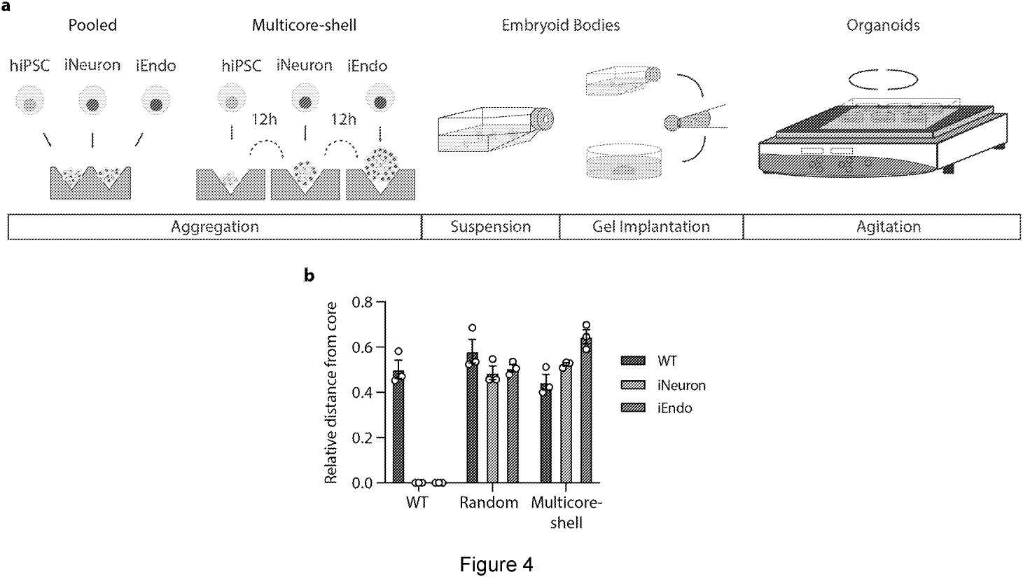

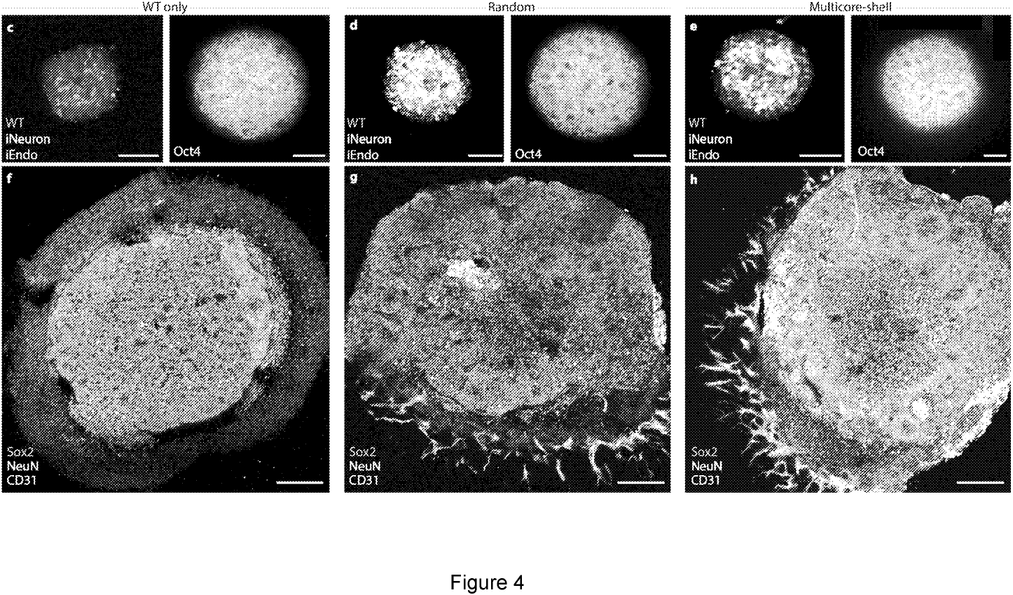

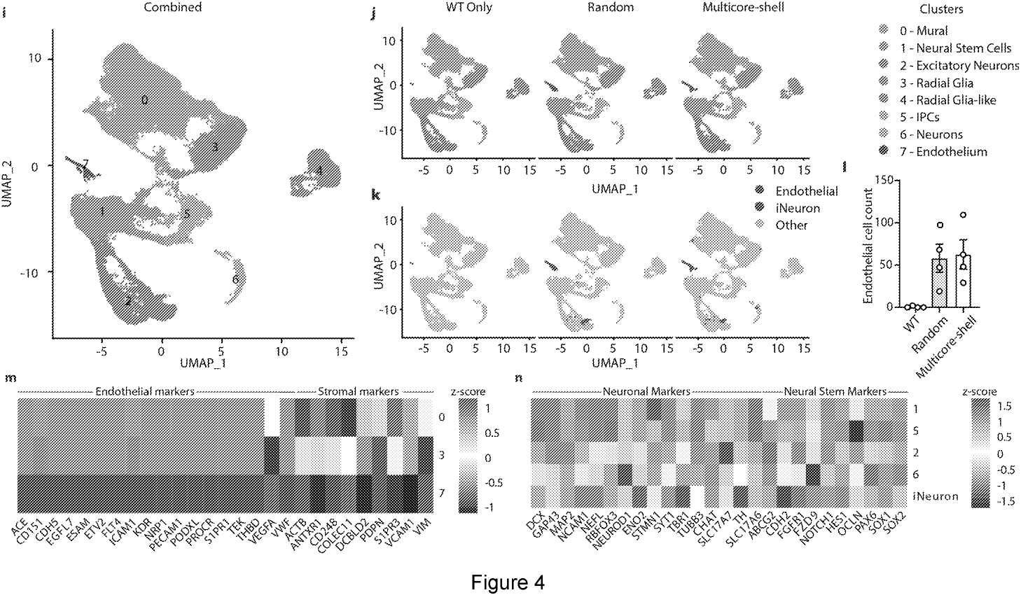

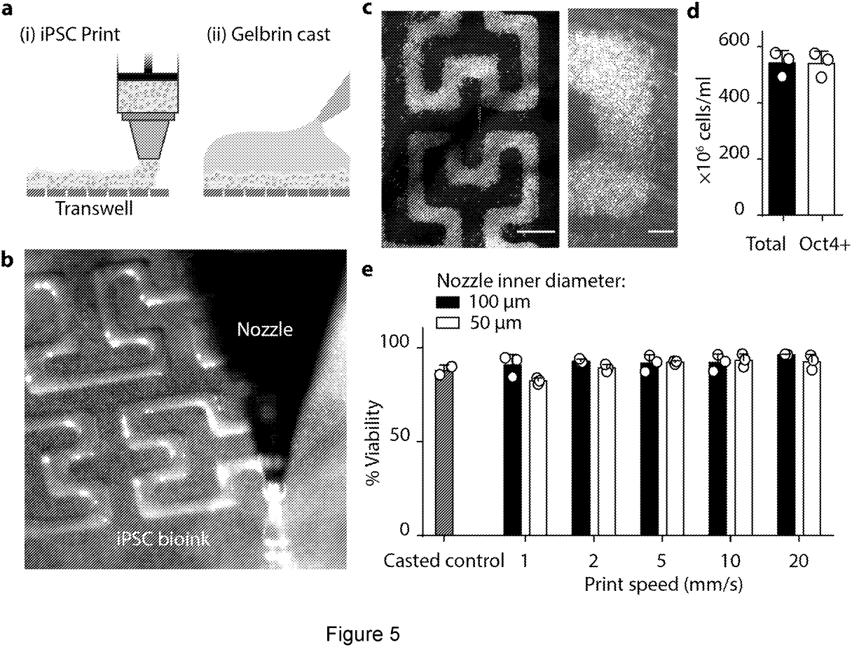

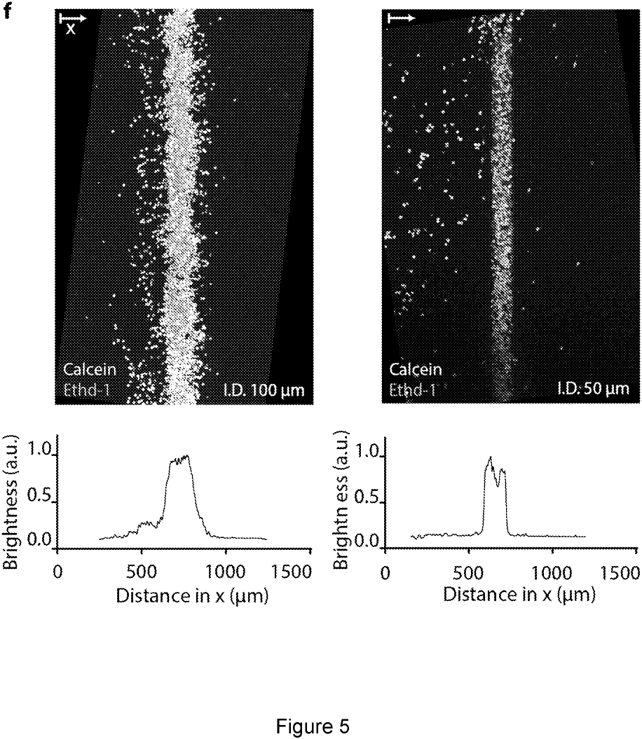

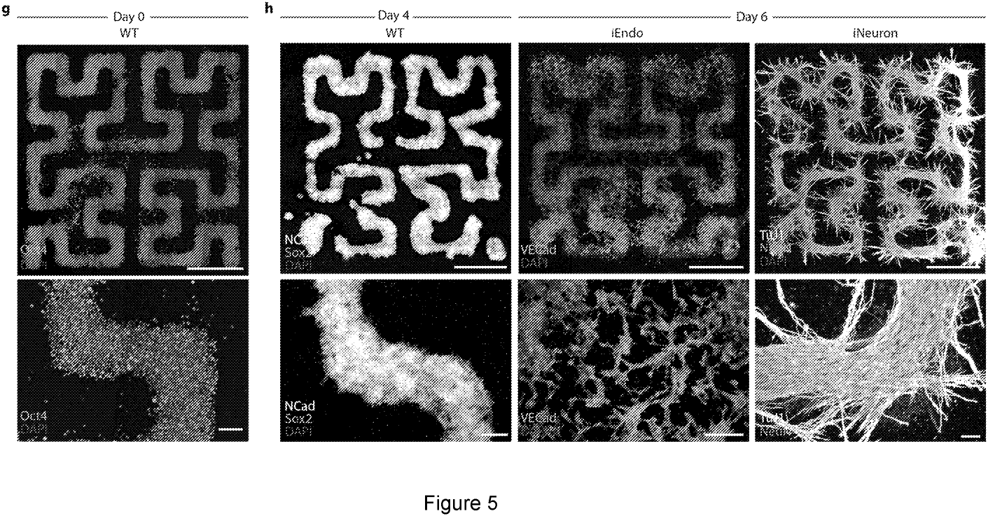

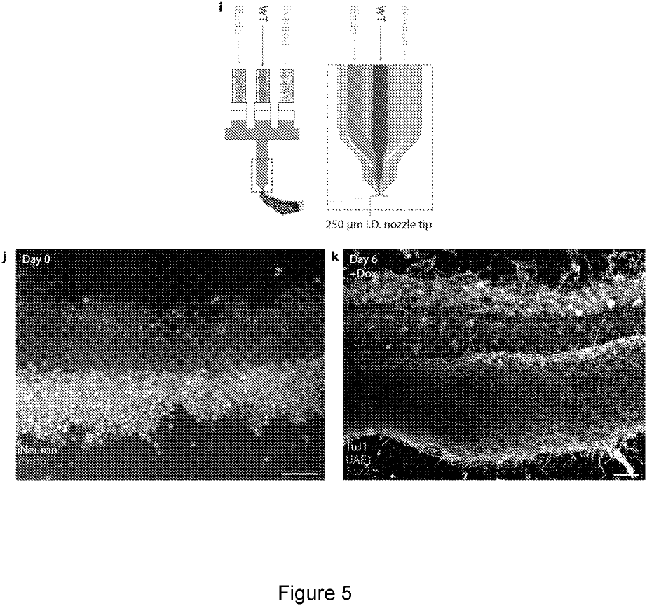

Statistics and reproducibility. FIGS. 2g-l, n=3 independent biological replicates from cells differentiated from different passages; unpaired two-tailed T-test, g, not significant, h, P=7.06x 10⁻⁷, i, P=3.53x 10⁻⁵. FIG. 3j, n=5 optical sections from single whole mount WT and WT+iEndo organoids; unpaired two-tailed T-test, P=4.23x 10⁻⁵. FIG. 3l, n=6 organoids from 3 independent batches. FIG. 4b, n=3 organoids from 3 independent batches. FIG. 5d, n=3 independent bioink batches. FIG. 5e, n=2 (casted control), n=3 (otherwise) independent batches of bioinks. FIG. S7g, P = 0.0169 between WT-only and randomly pooled organoids, P = 0.0286 between randomly pooled and multicore-shell organoids (n = 72 WT ventricles, n = 10 randomly pooled ventricles, n = 30 multicore-shell ventricles, unpaired two-tailed t-test).

### Data Availability

The datasets generated or analyzed during the current study are available from the corresponding author on reasonable request.

### Code Availability

The code generated during the current study is available from the corresponding author on reasonable request.

### Biological Materials Availability

The PBAN001 plasmid and hiPSC lines (PGP1, iEndo and iNeuron) are available upon request and execution of appropriate Material Transfer Agreements from Prof. George Church’s laboratory.

### Results

### Programming Stem Cell Differentiation

The derivation of neural tissue from hiPSCs serves as an ideal example to assess the feasibility of OlD, given that pluripotent cells are efficiently differentiated into neuroectoderm in a dual-SMAD inhibiting medium³⁰ (FIG. 2).

Three different Personal Genome Project 1 (PGPI) hiPSC lines were tested, one WT line and two inducible-TF lines, in identical media conditions. WT PGPl cells form neural stem cells were cultured in neural induction medium (NIM) containing TGF-β- and BMP4-pathway small molecule inhibitors, as previously reported³¹ (FIG. 2a). For the inducible-TF lines, a PiggyBac transposon vector was utilized to incorporate an all-in-one Tet-On system that enables the rapid and highly efficient doxycycline-induced upregulation of transcription factors. To generate inducible endothelial (iEndo) cells, the transcription factor ETV2 was overexpressed, which is known to drive rapid and efficient directed differentiation of pluripotent cells to vascular endothelial cells²³ (FIG. 2b). To generate inducible neurons (iNeuron), NGNl was upregulated, as single- or co-expression of neurogenins is known to rapidly generate neurons from stem cells^{24,32} (FIG. 2c).

The differentiation of WT and inducible-TF hiPSC lines in the absence (control) and presence of doxycycline was characterized. When cultured for six days in NIM, WT hiPSCs experienced a loss of pluripotency and efficiently differentiated into a neural stem cell fate in both cases, as indicated by the formation of characteristic polarized neural rosettes and the expression of neural cadherin (Ncad), paired-box gene 6 (Pax6), SRY-box transcription factor 2 (Sox2) and Nestin (FIG. 2d). Similarly, iEndo cells underwent media-driven differentiation to neural stem cells in the absence of doxycycline (FIG. 2e). However, in the presence of doxycycline, the overexpression of ETV2 drove the rapid differentiation to a vascular endothelial cell phenotype, as evidenced by the formation of a confluent cobblestone morphology, the expression of vascular endothelial cadherin (VECad), von-Willebrand factor (vWF), CD31, and the vascular endothelial growth factor receptor Neuropilin I (NRPl). iNeuron cells also differentiated into neural stem cells in the absence of doxycycline (FIG. 2f). In the presence of doxycycline, iNeurons formed neurons with a bipolar morphology and expressed neural markers Tuj1, microtubule associated protein 2 (MAP2) and neuronal nuclei (NeuN). When induced, both iEndo and iNeuron cells efficiently and rapidly differentiated, confirming the feasibility of OID (FIGS. 2g-l, FIG. 6). Indeed, surprisingly, the stark contrast between the differentiation of iEndo in the presence (99% vascular endothelium) and absence (94% neural stem cells) of doxycycline serves as a striking illustration of the orthogonality of external (media-driven) versus internal (TF-directed) cell differentiation.

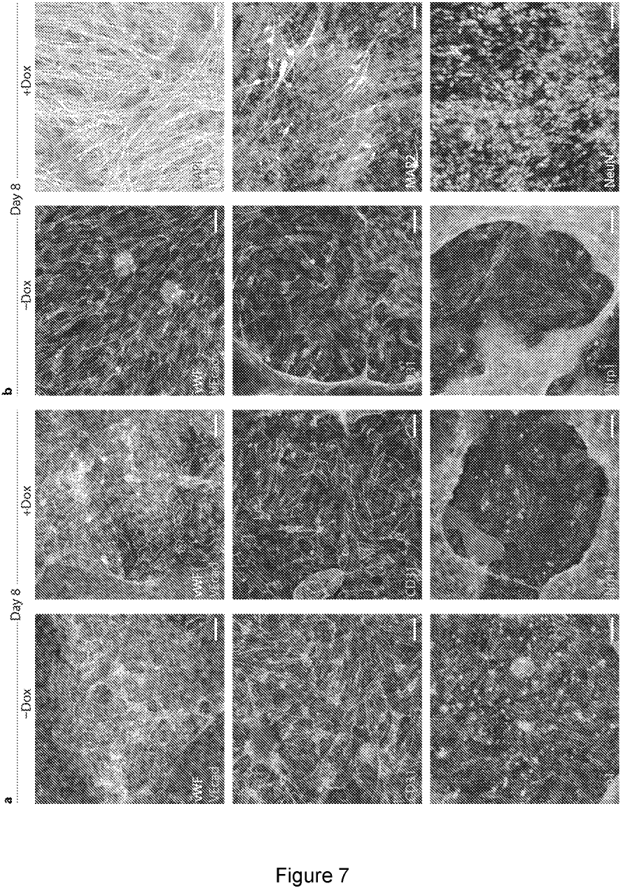

To assess the generalizability of OID, WT and iNeuron hiPSCs were subjected to media conditions that drive endothelial differentiation via an established protocol that uses GSK-3β inhibition to direct cells into mesoendoderm before applying growth factors to specify endothelium³³. After 8 days in differentiation, WT cells differentiated into endothelium expressing VEcad, vWF, CD31, and NRPl with a typical cobblestone morphology irrespective of the presence of doxycycline (FIG. 7(a)). Without doxycycline, iNeuron cells differentiated similarly into endothelium. However, in the presence of doxycycline, iNeuron cells differentiated into bipolar neurons that express Tuj1, MAP2, and NeuN (FIG. 7(b)). Taken together, these data clearly demonstrated that intracellular TF-driven differentiation can fully override the otherwise strong and specific extracellular differentiation cues.

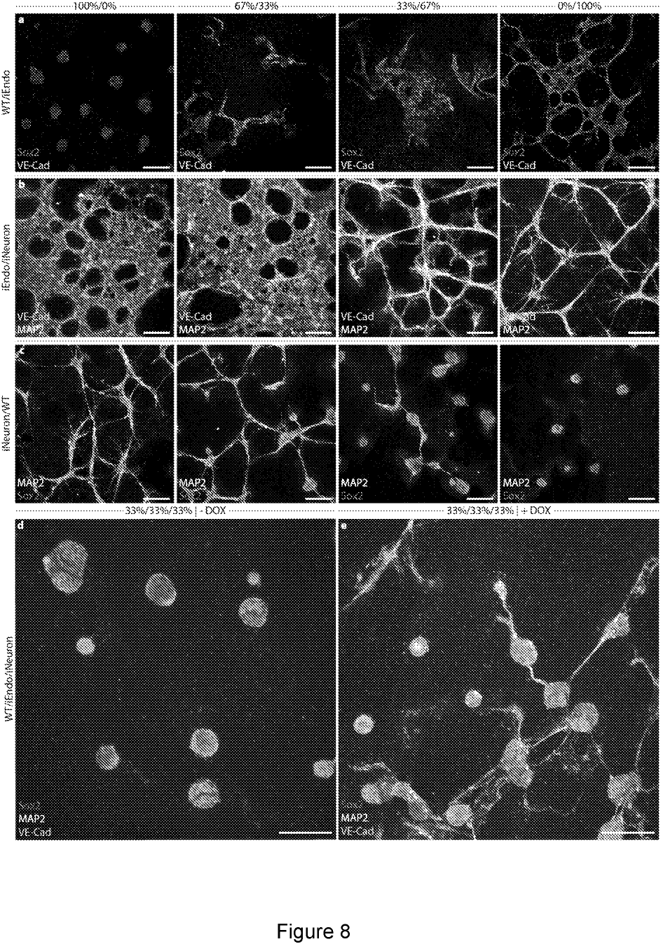

Next, it was investigated whether simultaneous OID of a mixed initial population of hiPSCs would give rise to a heterogeneous differentiated cell population with programmed composition. Specifically, different proportions of WT, iEndo, and iNeuron hiPSCs were seeded onto a Matrigel surface and cultured in NIM with doxycycline. It was observed that distinct multicellular populations form after six days in one-pot culture conditions (FIG. 8). Furthermore, under these conditions, iEndo cells self-assembled into a network-like microvasculature that is consistent with endothelial tubulogenesis assays. In contrast, WT cells formed neurospheres that rose above the surface of the gel, while iNeuron cells formed a network of neurites. Notably, when iEndo and WT cells were cultured together, the differentiated cells formed a distinct network pattern in which endothelial cells comprised the edges and neurospheres comprised the nodes. Using iEndo-mKate2 and WT-GFP labeled cells, it was confirmed that the vascular and neurosphere components were indeed composed of iEndo and WT cells, respectively (not shown). When iEndo and iNeuron cells were cultured together, the resulting endothelial and neuronal cells formed overlapping networks in which neurites extend along the vascular network.

To demonstrate simultaneous OID, all three cell lines were pooled together and cultured in NIM, and in the absence (control) and presence of doxycycline. Absent doxycycline, all three cell lines differentiated into neurospheres, with no visible VE-Cad or MAP2 staining. However, surprisingly and unexpectedly, in the presence of doxycycline, WT, iEndo, and iNeuron cells are differentiated into Sox2+ neurospheres, VE-Cad+ vascular endothelium, and MAP2+ neurons, respectively. Importantly, these results show that OID offers several distinct advantages over traditional organoid differentiation protocols, which typically aim to derive cells from a single germ layer^{13-15,20,21}.

### Programmable Cortical organOIDs

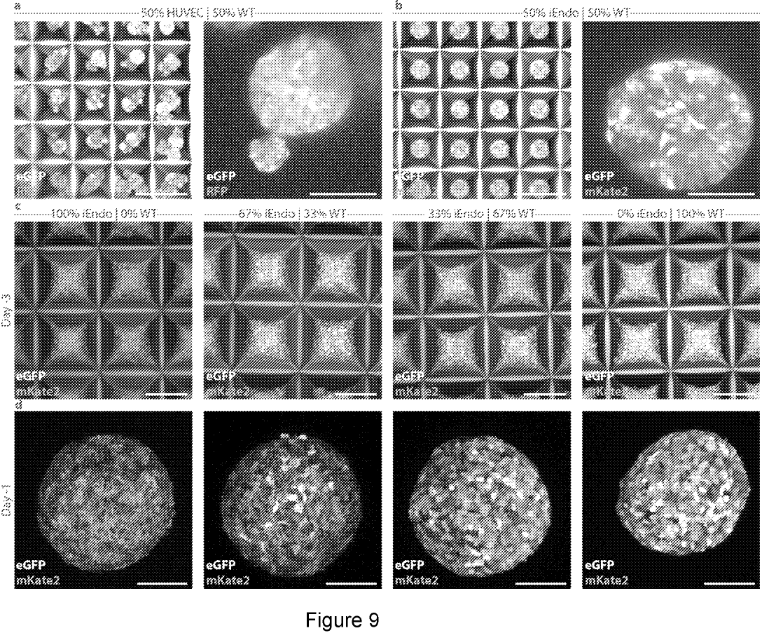

Having demonstrated that our platform is capable of programming stem cell differentiation with near-unity efficiency, we next addressed the challenge of organoid vascularization, since traditional cerebral (and cortical) organoid protocols fail to generate an embedded vascular network¹³⁻¹⁵. One naïve strategy to achieving this goal is to simply mix endothelial cells with hiPSCs to form a multicellular embryoid body that subsequently differentiates into the desired vascularized organoid. However, we find that human umbilical vein endothelial cells (HUVECs) mixed with WT hiPSCs in microwells will undergo phase separation within 24 h (FIG. 9(a)). This observation likely arises due to differences in cell adhesion³⁴, as endothelial cells and epithelial hiPSCs express different cell adhesion molecules. Importantly, when iEndo-mKate2 and WT-GFP hiPSCs are mixed and cultured in microwells, single embryoid bodies are formed with interspersed cells (FIG. 9(b), FIGS. 3b-c). By controlling the ratio of WT-to-iEndo cells seeded in each microwell, we can precisely tailor the resulting endothelium after their orthogonal induced differentiation to form organ-OlDs (FIGS. 9(c)-(d)). To illustrate this, we produced vascularized cortical organOIDs by pooling 67% WT hiPSCs and 33% iEndo cells into embryoid bodies that are cultured in dual-SMAD inhibiting media to direct dorsal forebrain formation. After 3 days in suspension culture, the embryoid bodies are embedded into a Matrigel-collagen extracellular gel droplet. At 10 days, the avascular WT-only organoids (control) retained a smooth border, while the organOIDs comprised of 2:1 ratio of WT-to-iEndo cells formed extensive sprouted vascular networks that penetrate into the surrounding gel, as marked by lectin and visualized using bright-field (FIG. 3d) and fluorescence microscopy (FIG. 3e). The complete absence of microvascular markers in the WT organoids is consistent with previous reports that cortical organoids generated by traditional differentiation protocols lack a vascular network.

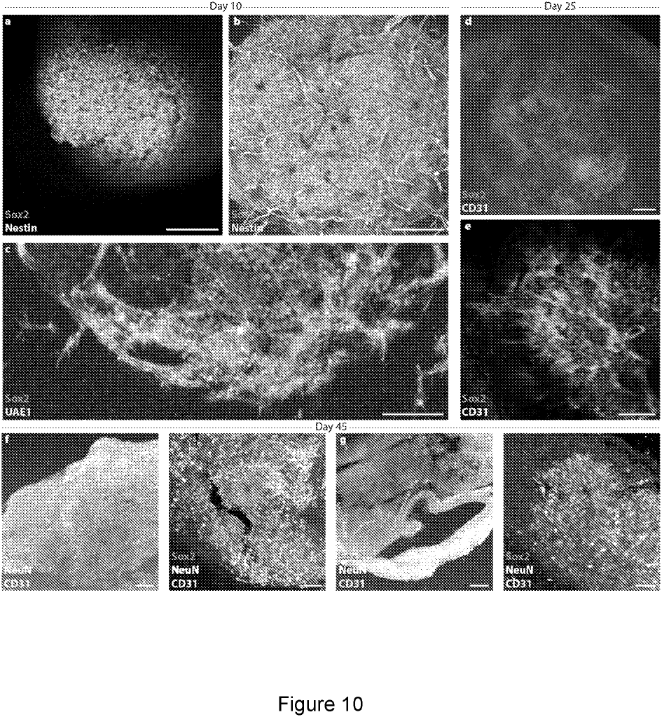

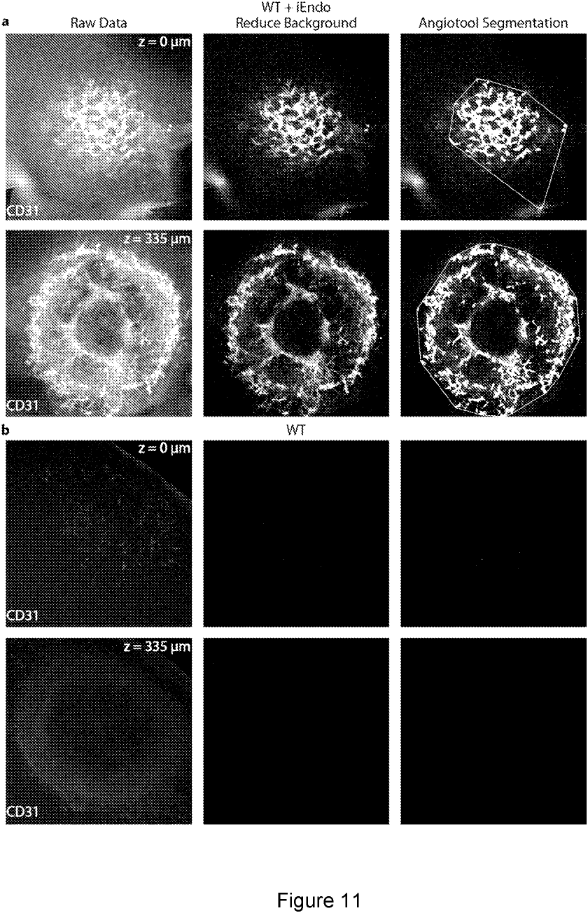

By pooling fluorescent mKate2-expressing iEndo cells, we confirmed that the embedded and sprouting vascular network observed within these organOIDs arises solely from those cells, as evidenced by the co-localization of CD3l and mKate2 (FIG. 3f). lntriguingly, we see evidence of Sox2+ neural stem cells colocalizing along the sprouting vasculature (arrowheads, FIG. 3f). This interaction is consistent with our observations in co-culture of neural stem cells and iEndo endothelium on Matrigel (FIG. 8) as well as known in vivo interactions of neural stem cells and endothelium in the neural stem cell niche in the subventricular zone³⁵. Notably, endothelial cells reside in between the rosette or ventricle-like structures, whereas the Sox2+ germinal zone-like structures are devoid of these cells, consistent with observed vascular patterns in neurodevelopment³⁶. After 25 days in culture, the vascularized cortical organOIDs developed NeuN positive neural populations (FIG. 3g, FIG. 10) and only organOIDs containing 33% iEndo cells possessed an embedded vascular network (FIG. 3h). Using whole-mount immunofluorescence of optically cleared organoids, we confirmed that this vascular network exists both at the organOID surface and within their core (FIG. 3i). Embedded vascular networks are imaged in multiple slices within the cleared organoids, revealing a sharp difference between the WT-only and the WT+iEndo cortical organoids (FIG. 3j, FIG. 11). As expected, there is no detectable vasculature within the WT-only organoids. However, by day 45 of culture, WT+iEndo cortical organOIDs developed ventricle-like architectures that are surrounded by neural cell bodies, neurites, and pervasive vascular network (FIG. 3k, FIG. 10). Using quantitative reverse transcription polymerase chain reaction (RT-qPCR), we find that these vascularized cortical organOIDs have significantly enhanced expression of vasculature-related genes (CDH5, PECAM l), while neural stem cell, neural, and pluripotent gene expression remain similar to the WT-only (control) organoids (FIG. 3l). This result demonstrates that OIDOID promotes vascular network formation without adversely affecting the neural phenotype within these organOIDs.

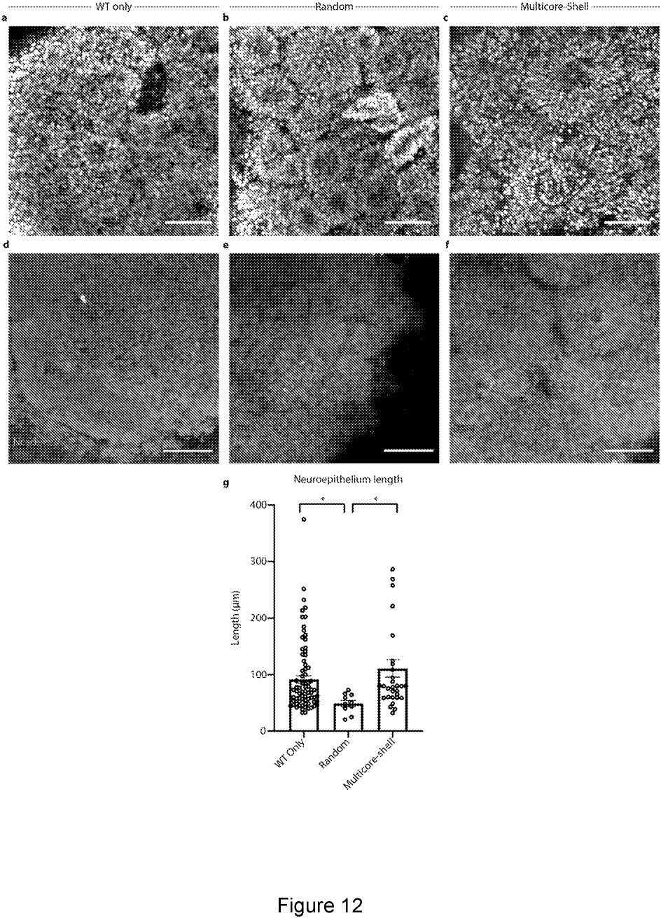

Moving beyond the random incorporation of iEndo cells into pooled embryoid bodies to form vascularized organoids, we then sought to create spatially patterned cortical organOIDs. We combined WT, iEndo, and iNeuron cells in a multicore-shell organOID architecture that is reminiscent of a developing brain, i.e., a germinal zone (GZ) (inner core), surrounding neurons (middle layer), and an encapsulating perineural vascular plexus (outer shell) (FIG. 4a). Additionally, the incorporation of iNeuron cells within these organOIDs enables faster neurogenesis when compared to cortical organoids produced by traditional protocols, which typically require ~30 days of culture to form a NeuN+ neuronal population³⁷. Stepwise addition of hiPSCs in a U-well plate has previously been shown to generate a Janus embryoid body that can be used in conjunction with an inducible Sonic Hedgehog-expressing hiPSC line to encourage dorsoventral axis formation³⁸. By using V-shaped wells, we created organoids with a radial-symmetric multicore-shell architectures, rather than two hemispherical compartments seen for Janus organoids assembled in U-wells (FIG. 4b). Embryoid bodies formed from WT-only, randomly pooled {WT, iNeuron, iEndo} triple populations, and step-wise multicore-shell organoids all formed cohesive Oct4+ embryoid bodies after one day in culture (FIGS. 4c-e). Consistent with other cortical organoid protocols^{37,39}, those containing WT-only cells contained few neurons at day 10, as evidenced by a minimal amount of NeuN expressing cells (FIG. 4f). In contrast, the incorporation of iNeuron cells in both random and multicore-shell organOIDs resulted in a large NeuN+ neuronal population by day 10. These neuronal populations surround the ventricle-like structures within the organOIDs, forming a neuron-rich layer that resides more superficially than the deeper GZs. We also find clusters of NeuN+ neurons deep in the center of randomly incorporated organOIDs that are absent in multicore-shell patterned organOIDs (FIGS. 4g-h), suggesting that the initial spatial patterning imposed by stepwise aggregation informs later layered organization within cortical organoids. Furthermore, a pervasive CD3l+ vascular network is present throughout both randomly patterned and multicore-shell patterned organoids. Orthogonal induced differentiation of patterned multicore-shell embryoid bodies gives rise to cortical organOIDs with larger ventricle-like structures, as measured by the length of apical N-cadherin+ neuroepithelium, when compared to those that form in randomly pooled embryoid bodies, and are similar in size to those found in WT-only organoids. This suggests that neural self-assembly processes can be preserved in organOIDs by imposing spatial patterning (FIG. 12).

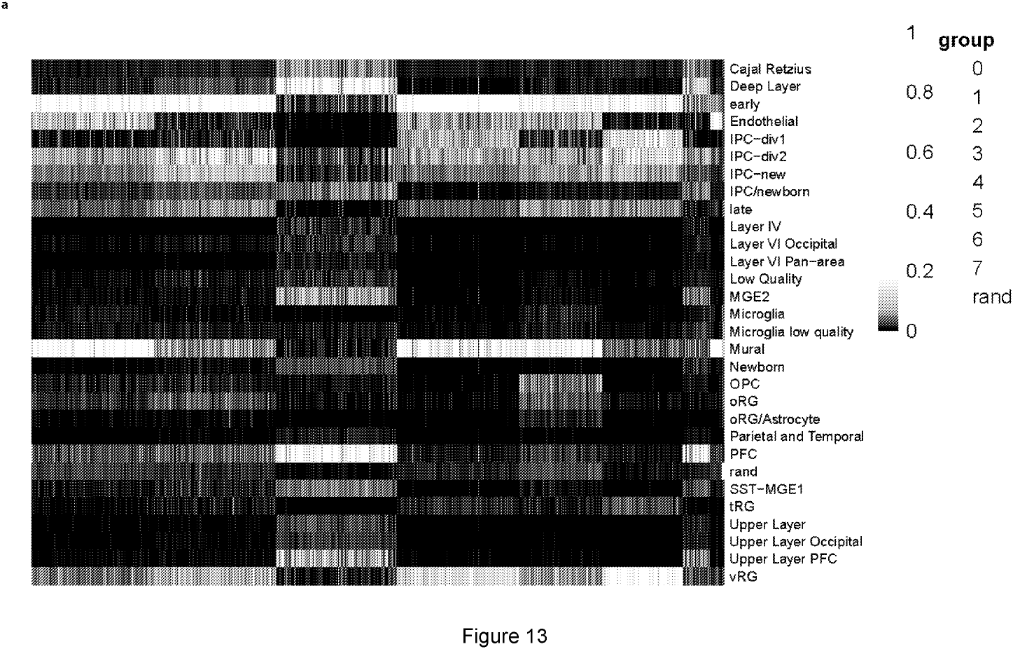

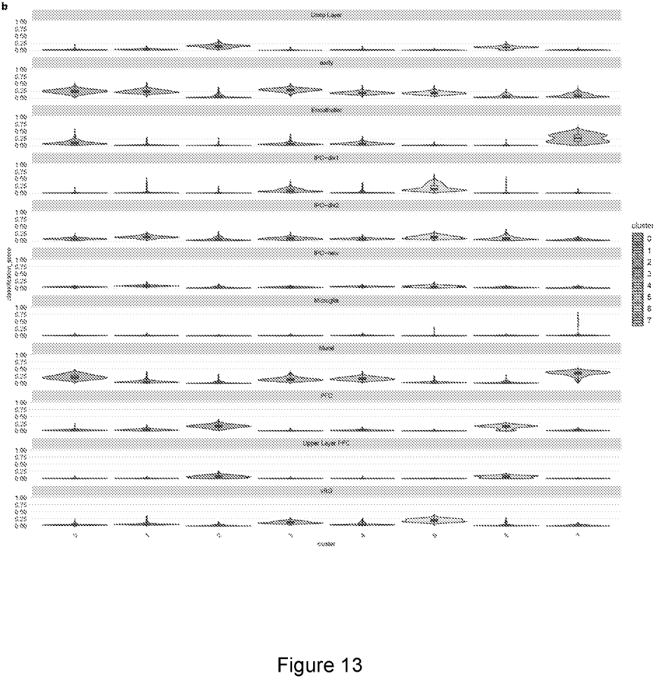

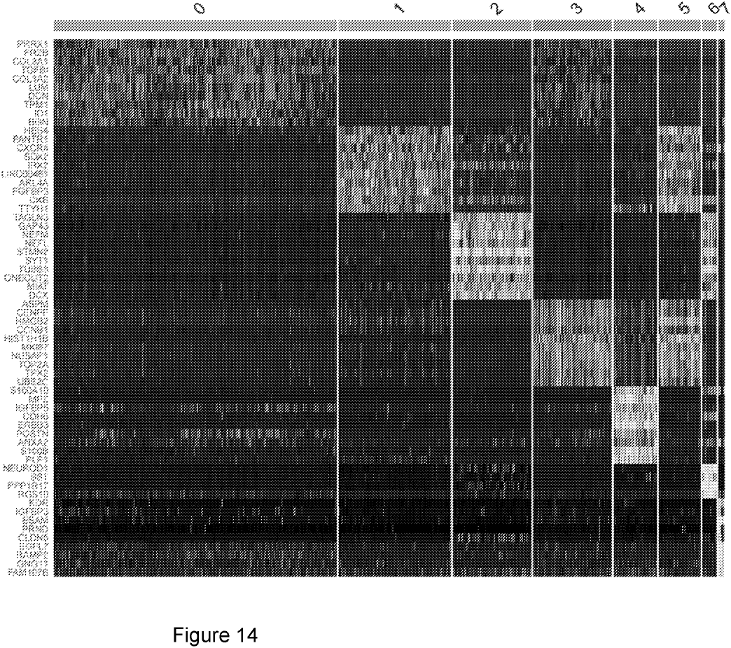

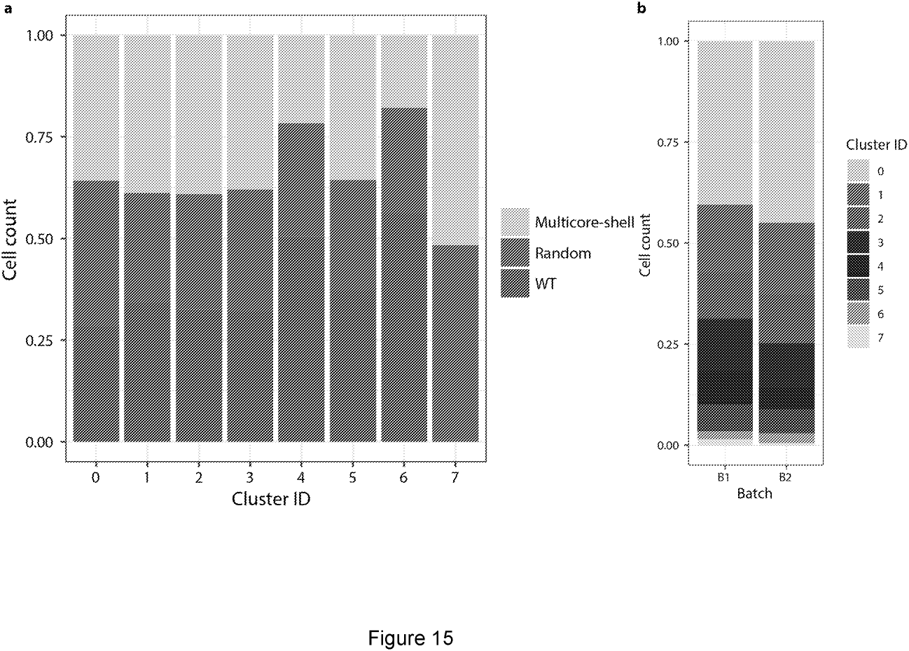

We next employed single-cell RNA sequencing (scRNA-seq) to investigate the cellular composition of day 25 organoids using the 10x Genomics™ pipeline (FIGS. 4i-j). To annotate the clusters, we employed the quantitative classification tool SingleCellNet⁴⁰ (FIG. 13) to compare primary brain tissue⁴¹ to our single cell datasets and plotted the top 10 most significantly differentially expressed genes in each cluster for validation (FIG. 14). We found that hiPSCs without a dox-inducible transgene (WT cells) differentiated into clusters of neural stem cells (cluster l), excitatory/inhibitory neurons (cluster 2), radial glia (clusters 3 & 4), intermediate progenitor cells (cluster 5), and neurons (cluster 6) (FIG. 4j). Cluster 0 is classified as cells of a non-neural lineage, with significant upregulation of mural cell markers such as COL3A l and CALDI⁴², which is consistent with previous observations of migratory stromal cells emerging from cerebral organoids that are embedded in Matrigel¹⁵. Endothelial cells (cluster 7) are nearly absent from WT-only organoids, but notably increase in both random and multicore-shell organOIDs as measured by total cell number (FIGS. 4(k)-(l), FIG. 15(a)). Furthermore, the cells in cluster 7 exhibit significant upregulation of endothelial markers compared to the clusters that exhibited some mesodermal lineage markers, clusters 0 and 3, including CD3l (PECAM l) and CDH5 (VE-cadherin) that are significantly enriched with respect to other clusters, strongly suggesting that cluster 7 is endothelium derived from iEndo cells (FIG. 4m). Finally, we investigated the identity of iNeurons in organOID culture. To ensure accurate tracking of neuronal-like cells we placed a barcode on the 3′ end of the transgene cassette. We found that iNeurons are transcriptionally most similar to mature neurons (FIG. 4n).

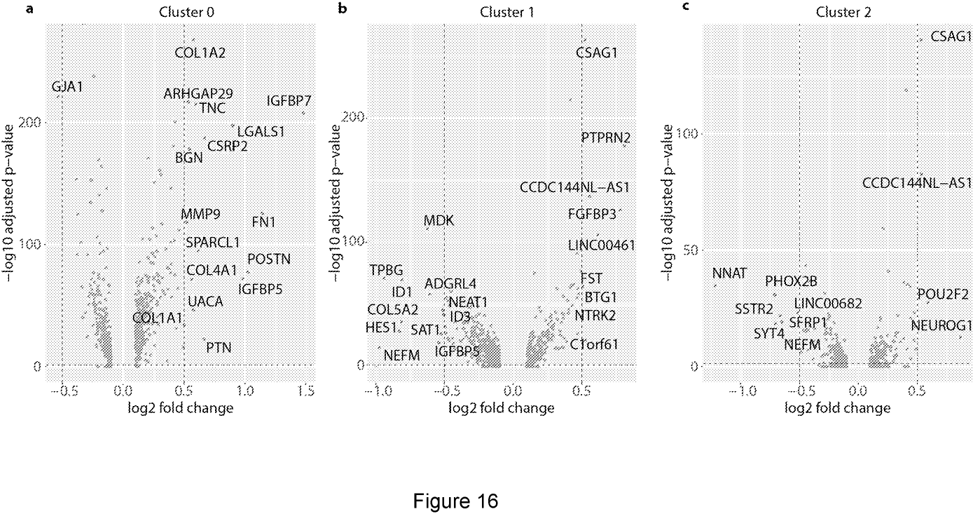

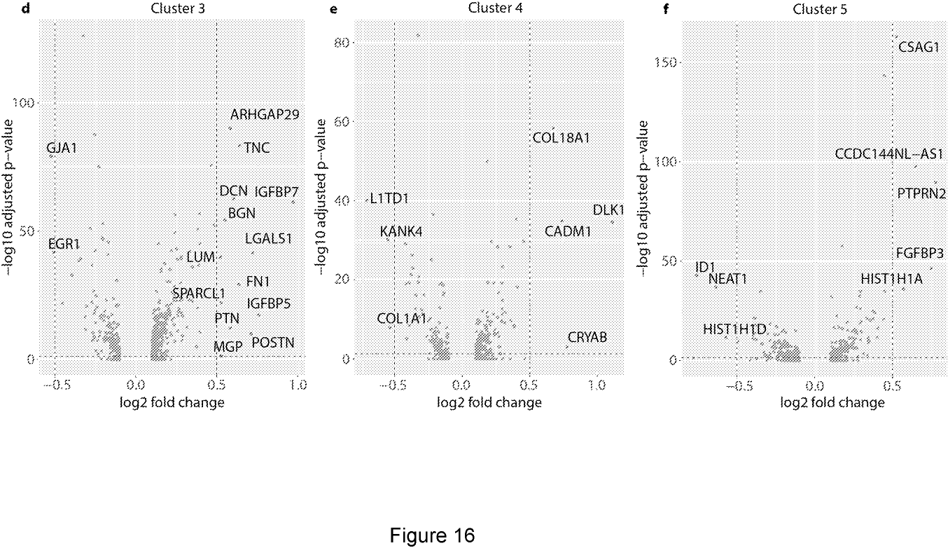

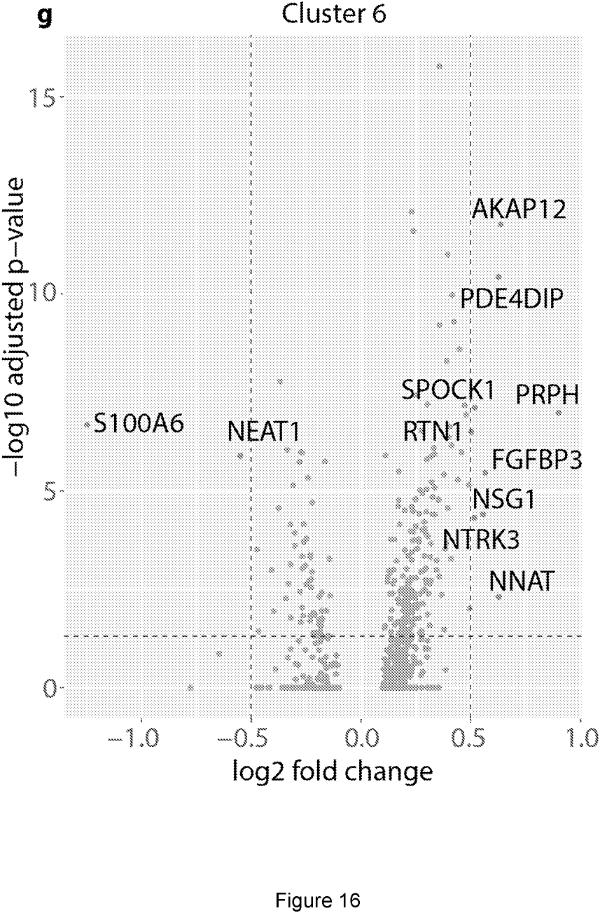

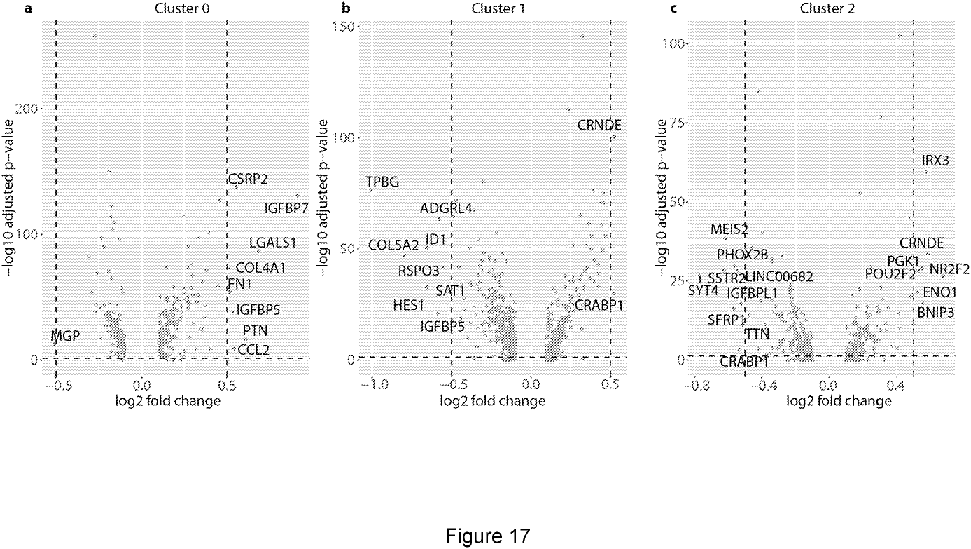

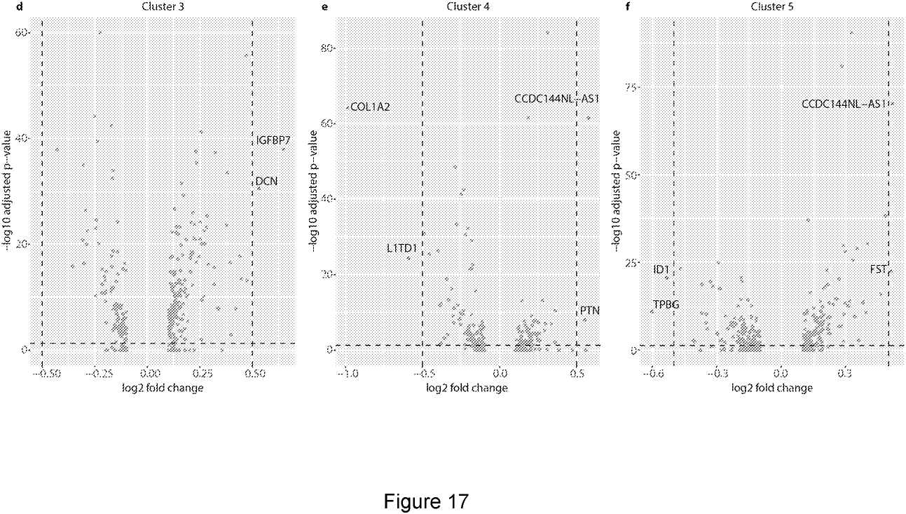

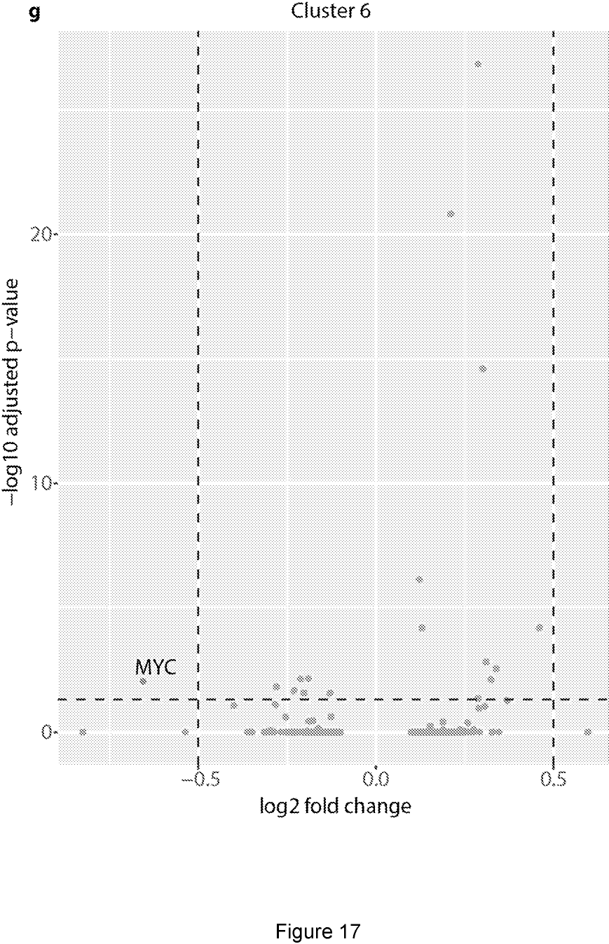

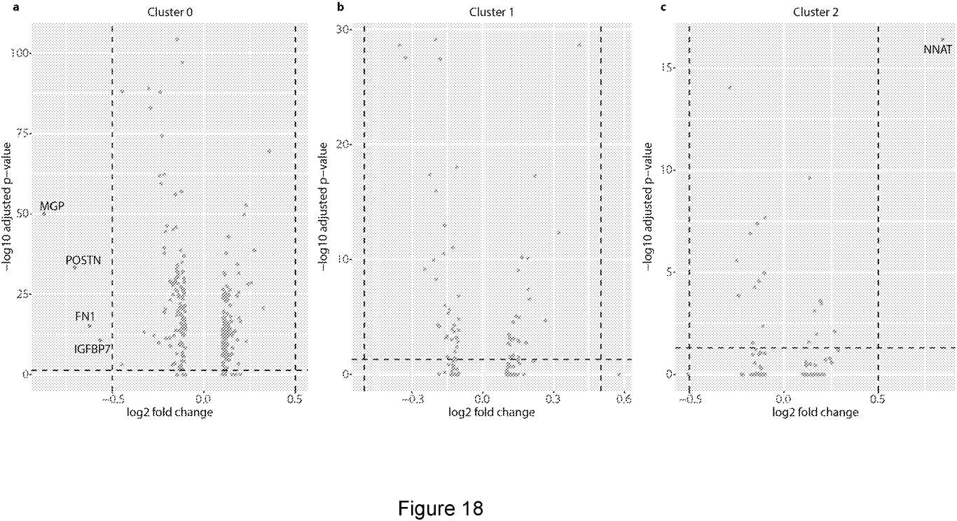

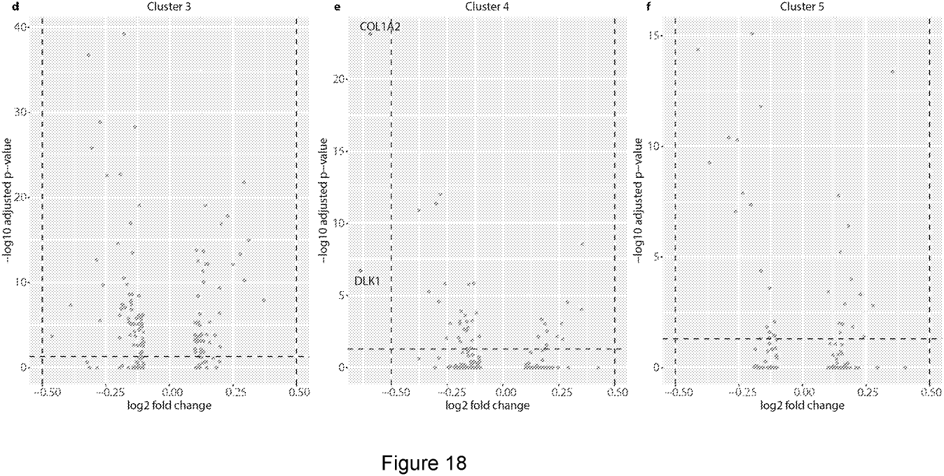

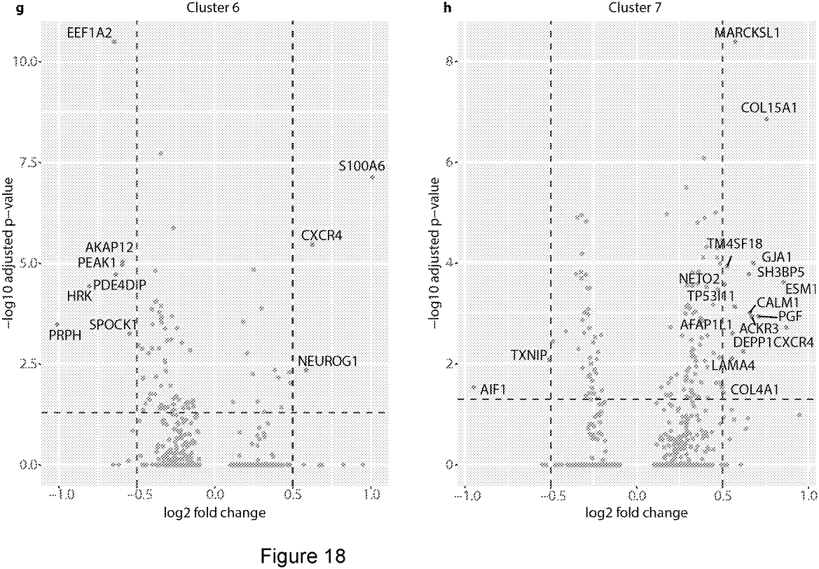

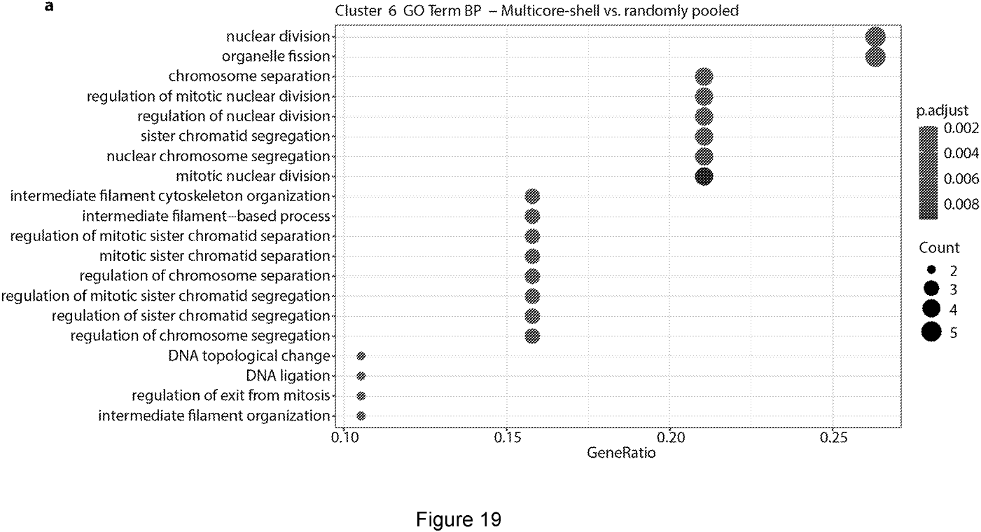

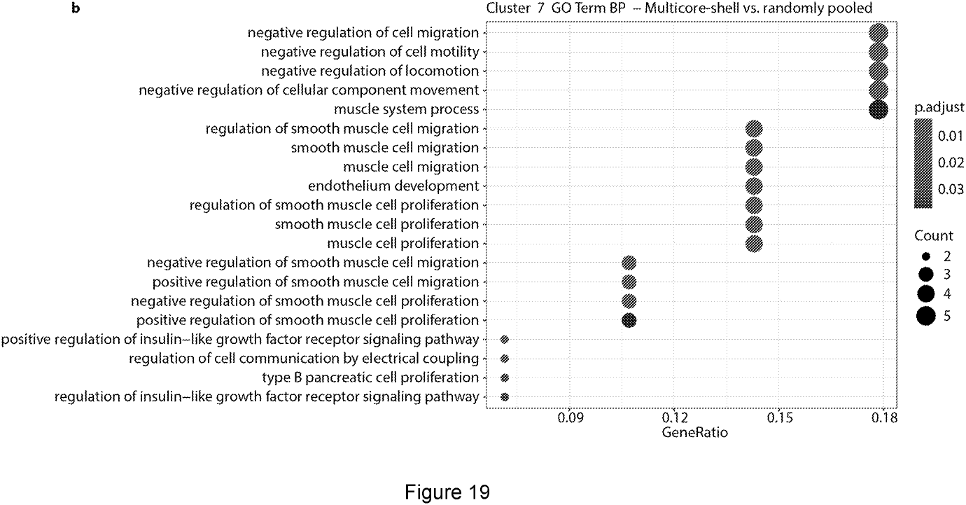

We then used our scRNA-seq data to compare the cell populations found in WT-only, randomly pooled, and multi-core shell organoids. Differential gene analysis on a cluster-by-cluster basis between all three organoids reveals that gene expression remains consistent for all clusters across all conditions and replicates (FIG. 15(b), and FIGS. 16-18). We found 33 genes with statistically significant differences in expression between randomly pooled and multicore shell organOIDs across all genes (FIG. 18). Specifically, only 7 genes were significantly differentially expressed in random and multicore-shell organOIDs among clusters 0-5. Gene ontology analysis of remaining 26 differentially expressed genes in clusters 6 and 7 of randomly pooled and multicore shell organoids shows an enrichment of genes associated with cell division in the neuronal cluster 6 and an enrichment of genes associated with cell migration in the endothelial cluster 7 (FIG. 19). These findings suggest that inducible cell lines can be added into existing cortical organoid protocols to generate additional specific cell types without major alterations to existing protocols. Thus, our OID platform can be used to enhance traditional organoid differentiation protocols by programming cell fate via deterministic design principles.

### Programmable 3D Neural Tissues via Bioprinting

Most bioprinting methods produce multicellular tissues by printing inks composed of different primary human cells suspended in hydrogel matrices. Recently, human mesenchymal stem cells (hMSCs)⁴³ and hiPSC^{12,44-47}-laden inks have been printed and differentiated in situ to form 3D tissue constructs. However, these stem-cell derived tissues contained cell types defined solely by media cues. By leveraging the proliferative capacity of hiPSCs along with the programmability afforded by our OID platform, single- and patterned multicellular 3D human tissues can be created with high cell densities. As a simple demonstration, we first created concentrated, matrix-free hiPSC bioinks by pelleting single-cell suspensions of hiPSCs and then directly printing these pluripotent inks onto transwell membranes (FIGS. 5a-c). The printed filamentary features, which contain a remarkably high cellular density exceeding 5×10⁸ Oct4+ cells per ml (FIG. 5d), are subsequently encased in a gelatin-fibrinogen ECM to support the tissue and enable 3D cell migration. Through calcein-AM/ethidium homodimer-2 Live/Dead assays of matrix-free hiPSC inks printed through 50 µm and 100 µm tapered nozzles at speeds of up to 20 mm/s, we confirm high cell viability akin to that observed for casted (control) tissues (FIG. 5e). Interestingly, we observe improved cell viability at higher print speeds, suggesting that shear forces have little effect over the range of printing conditions explored. We attribute the reduced cell viability observed at lower printing speeds to water evaporation from the printed cell-laden inks prior to encasing them within the gelatin-fibrinogen ECM. The pluripotent filaments are printed with a high resolution, with widths measuring 132 µm and 182 µm (FWHM) for bioinks printed using 50 µm and 100 µm nozzles, respectively (FIG. 5f). Next, we printed planar patterns in the form of third-order pseudo-Hilbert curves composed of filamentary features produced from WT, iEndo, or iNeuron inks (FIGS. 5g-h). Printed WT filaments exhibited a slight compaction when differentiated in NIM containing doxycycline, forming a neuroectoderm filament with small Ncad+ ventricle-like structures or rosettes punctuating its length. By contrast, printed and differentiated (iEndo) filaments exhibited vasculogenesis, resulting in the formation of a microvascular network over time. Finally, printed iNeuron filaments differentiated into densely packed neurons (NeuN+ cells) that formed a pervasive network of protruding Tuj1 + neurites.

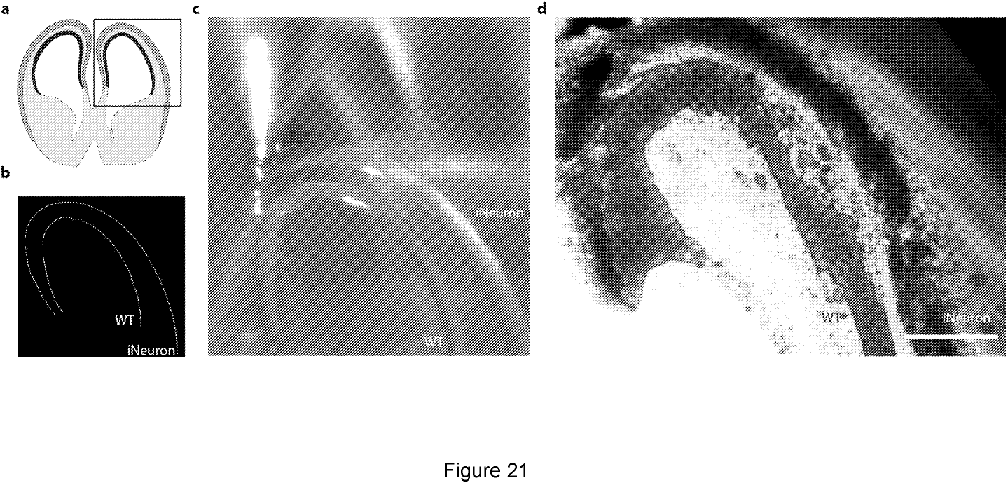

As a final demonstration, we created multicellular tissue architectures by co-printing three bioinks composed of WT, iEndo, and iNeuron hiPSCs, respectively (FIG. 5i, FIG. 20). The printhead contains three independent ink delivery channels that merge shortly upstream of the 250 µm-wide nozzle. Under laminar flow, these three inks converge to form tri-layer filaments that exit the nozzle. Using fluorescent dye-labeled WT, iEndo and iNeuron hiPSCs, we showed that this multimaterial printhead can successfully generate multicellular pluripotent filaments in layered architectures with high fidelity with a filament width of 269 µm (FWHM) (FIG. 5j). After printing, we added doxycycline to induce the simultaneous differentiation of neural stem cells, vascular endothelium, and neurons and observed that the layered architecture is preserved over 6 days of culture. (FIG. 5k). Finally, using this integrated OID-based bioprinting platform, we recapitulated the geometry of a developing human dorsal forebrain coronal section by co-printing densely cellular WT and iNeuron hiPSC inks (FIGS. 21(a)-(c)). After printing, the printed inks undergo OID to form a neuroectoderm and overlying neuron-dense layer (FIG. 21(d)). As new protocols for deriving specific neuronal cell types emerge, our platform will enable patterning multilayer cortical architectures from an even broader array of inducible-TF hiPSCs.

### Acknowledgements

This research was sponsored by the National Human Genome Research Institute of the NIH under award number RMIHG008525 and by the Vannevar Bush Faculty Fellowship Program, sponsored by the Basic Research Office of the Assistant Secretary for Defense for Research and Engineering through the Office of Naval Research Grant N00014-16-1-2823. A.L. received fellowship support from the Charles Stark Draper Laboratory. The authors also thank T. Ferrante and the Harvard Center for Biological Imaging for microscopy infrastructure and support, S. Uzel, S. Han, and M. Mata for experimental assistance, and J. Coppeta for useful discussions. The authors have submitted a patent application associated with this work.

### REFERENCES

1. Yin, X. et al. Engineering stem cell organoids. Cell Stem Cell 18, 25-38 (2016).

2. Takebe, T. & Wells, J. M. Organoids by design. Science 364, 956-959 (2019).

3. Laurent, J. et al. Convergence of microengineering and cellular self-organization towards functional tissue manufacturing. Nat. Biomed. Eng. 1, 939-956 (2017).

4. Brassard, J. A. & Lutolf, M. P. Engineering Stem Cell Self-organization to Build Better Organoids. Cell Stem Cell 24, 860-876 (20l9).

5. Lancaster, M. A. & Knoblich, J. A. Organogenesis in a dish: Modeling development and disease using organoid technologies. Science 345, 1247125-1247125 (2014).

6. Clevers, H. Modeling Development and Disease with Organoids. Cell l65, 1586-1597 (2016).

7. Lee, A. et al. 3D bioprinting of collagen to rebuild components of the human heart. Science 365, 482-487 (2019).

8. Lee, W. et al. Multi-layered culture of human skin fibroblasts and keratinocytes through three-dimensional freeform fabrication. Biomaterials 30, 1587-1595 (2009).

9. Kolesky, D. B. et al. 3D Bioprinting of Vascularized, Heterogeneous Cell-Laden Tissue Constructs. Adv. Mater. 26, 3124-3130 (2014).

10. Skylar-Scott, M. A. et al. Biomanufacturing of organ-specific tissues with high cellular density and embedded vascular channels. Sci. Adv. 5, eaaw2459 (2019).

11. Kang, H.-W. et al. A 3D bioprinting system to produce human-scale tissue constructs with structural integrity. Nat. Biotechnol. 34, 312-319 (2016).

12. Gu, Q., Tomaskovic-Crook, E., Wallace, G. G. & Crook, J. M. 3D Bioprinting Human Induced Pluripotent Stem Cell Constructs for In Situ Cell Proliferation and Successive Multilineage Differentiation. Adv. Healthc. Mater. 6, 1-11 (2017).

13. Yoon, S.-J. et al. Reliability of human cortical organoid generation. Nat. Methods 16, 75-78 (20l9).

14. Velasco, S. et al. Individual brain organoids reproducibly form cell diversity of the human cerebral cortex. Nature 570, 523-527 (20l9).

15. Lancaster, M. A. et al. Cerebral organoids model human brain development and microcephaly. Nature 501, 373-379 (2013).

16. Takasato, M. et al. Kidney organoids from human iPS cells contain multiple lineages and model human nephrogenesis. Nature 526, 564-568 (20l5).

17. Morizane, R. et al. Nephron organoids derived from human pluripotent stem cells model kidney development and injury. Nat. Biotechnol. 33, 1193-1200 (20l5).

18. Homan, K. A. et al. Flow-enhanced vascularization and maturation of kidney organoids in vitro. Nat. Methods l6, 255-262 (20l9).

19. Eiraku, M. et al. Self-organizing optic-cup morphogenesis in three-dimensional culture. Nature 472, 51-56 (2011).

20. Kelava, l. & Lancaster, M. A. Stem Cell Models of Human Brain Development. Cell Stem Cell l8, 736-748 (2016).

21. Phipson, B. et al. Evaluation of variability in human kidney organoids. Nat. Methods 16, 79-87 (20l9).

22. Morita, R. et al. ETS transcription factor ETV2 directly converts human fibroblasts into functional endothelial cells. Proc. Natl. Acad. Sci. U. S. A. 112, 160-165 (20l5).

23. Elcheva, l. et al. Direct induction of haematoendothelial programs in human pluripotent stem cells by transcriptional regulators. Nat. Commun. 5, 4372 (2014).

24. Busskamp, V. et al. Rapid neurogenesis through transcriptional activation in human stem cells. Mol. Syst. Biol. 10, 760 (2014).

25. Sekiya, S. & Suzuki, A. Direct conversion of mouse fibroblasts to hepatocyte-like cells by defined factors. Nature 475, 390-393 (2011).

26. Huang, P. et al. Induction of functional hepatocyte-like cells from mouse fibroblasts by defined factors. Nature 475, 386-389 (2011).

27. Guye, P. et al. Genetically engineering self-organization of human pluripotent stem cells into a liver bud-like tissue using Gata6. Nat. Commun. 7, 10243 (2016).

28. Cakir, B. et al. Engineering of human brain organoids with a functional vascular-like system. Nat. Methods l6, 1169-1175 (20l9).

29. Ng, A. H. M. et al. A comprehensive library of human transcription factors for cell fate engineering. Nat. Biotechnol. (2020) doi: l0. 1038/s41587-020-0742-6.

30. Kim, D.-S. et al. Robust Enhancement of Neural Differentiation from Human ES and iPS Cells Regardless of their Innate Difference in Differentiation Propensity. Stem Cell Rev. Reports 6, 270-281 (2010).

31. Chambers, S. M. et al. Highly efficient neural conversion of human ES and iPS cells by dual inhibition of SMAD signaling. Nat. Biotechnol. 27, 275-280 (2009).

32. Matsushita, M. et al. Neural differentiation of human embryonic stem cells induced by the transgene-mediated overexpression of single transcription factors. Biochem. Biophys. Res. Commun. 490, 296-301 (2017).

33. Gu, M. Efficient Differentiation of Human Pluripotent Stem Cells to Endothelial Cells. Curr. Protoc. Hum. Genet. 98, 1-16 (2018).

34. Steinberg, M. S. On the mechanism of tissue reconstruction by dissociated cells. l. Population kinetics, differential adhesiveness. and the absence of directed migration. Proc. Natl. Acad. Sci. U. S. A. 48, 1577-1582 (1962).

35. Goldberg, J. S. & Hirschi, K. K. Diverse roles of the vasculature within the neural stem cell niche. Regen. Med. 4, 879-897 (2009).

36. Takahashi, T. et al. Angiogenesis in the Developing Spinal Cord: Blood Vessel Exclusion from Neural Progenitor Region Is Mediated by VEGF and Its Antagonists. PLoS One 10, e01 161 19 (20l5).

37. Qian, X. et al. Brain-Region-Specific Organoids Using Mini-bioreactors for Modeling ZIKV Exposure. Cell l65, 1238-1254 (2016).

38. Cederquist, G. Y. et al. Specification of positional identity in forebrain organoids. Nat. Biotechnol. 37, 436-444 (20l9).

39. Pasca, A. M. et al. Functional cortical neurons and astrocytes from human pluripotent stem cells in 3D culture. Nat. Methods 12, 671-678 (20l5).

40. Tan, Y. & Cahan, P. SingleCellNet: A Computational Tool to Classify Single Cell RNA-Seq Data Across Platforms and Across Species. Cell Syst. 9, 207-2 13.e2 (20l9).

41. Bhaduri, A. et al. Cell stress in cortical organoids impairs molecular subtype specification. Nature 578, 142-148 (2020).

42. He, L. et al. Analysis of the brain mural cell transcriptome. Sci. Rep. 6, 1-13 (2016).

43. Kolesky, D. B., Homan, K. A., Skylar-Scott, M. A. & Lewis, J. A. Three-dimensional bioprinting of thick vascularized tissues. Proc. Natl. Acad. Sci. U. S. A. 113, 3179-3 I 84 (2016).

44. Kupfer, M. E. et al. In situ expansion, differentiation and electromechanical coupling of human cardiac muscle in a 3D bioprinted, chambered organoid. Circ. Res. (2020) doi:10.1161/CIRCRESAHA.119.316155.

45. Gu, Q. et al. Functional 3D Neural Mini-Tissues from Printed Gel-Based Bioink and Human Neural Stem Cells. Adv. Healthc. Mater. 5, 1429-1438 (2016).

46. Koch, L. et al. Laser bioprinting of human induced pluripotent stem cells - The effect of printing and biomaterials on cell survival, pluripotency, and differentiation. Biofabrication 10, (2018).

47. Li, Y. et al. 3D printing human induced pluripotent stem cells with novel hydroxypropyl chitin bioink: Scalable expansion and uniform aggregation. Biofabrication 10, (2018).

48. Seiler, C. Y. et al. DNASU plasmid and PSI:Biology-Materials repositories: resources to accelerate biological research. Nucleic Acids Res. 42, D I 253-D 1260 (2014).

49. Wiemann, S. et al. The ORFeome Collaboration: a genome-scale human ORF-clone resource. Nat. Methods 13, 191-192 (2016).

50. Lancaster, M. A. & Knoblich, J. A. Generation of cerebral organoids from human pluripotent stem cells. Nat. Protoc. 9, 2329-2340 (2014).

51. Birey, F. et al. Assembly of functionally integrated human forebrain spheroids. Nature 545, 54-59 (2017).

52. Renier, N. et al. Mapping of Brain Activity by Automated Volume Analysis of Immediate Early Genes. Cell l65, 1789-1802 (2016).

53. Schmid, B. et al. 3Dscript: animating 3D/4D microscopy data using a natural-language-based syntax. Nat. Methods l6, 278-280 (2019).

54. Zudaire, E., Gambardella, L., Kurcz, C. & Vermeren, S. A Computational Tool for Quantitative Analysis of Vascular Networks. PLoS One 6, e27385 (2011).

55. Hell, S., Reiner, G., Cremer, C. & Stelzer, E. H. K. Aberrations in confocal fluorescence microscopy induced by mismatches in refractive index. J. Microsc. 169, 391-405 (1993).

56. Bayer, S. A. & Altman, J. The Human Brain During the Third Trimester. (CRC Press LLC, 2003).

Throughout this specification various indications have been given as to preferred and alternative embodiments of the invention. However, the foregoing detailed description is to be regarded as illustrative rather than limiting and the invention is not limited to any one of the provided embodiments. It should be understood that it is the appended claims, including all equivalents, are intended to define the spirit and scope of this invention.

1. A method of generating a programmable multicellular organoid and/or a 3D organ-specific tissue, comprising:
culturing at least one genetically-engineered inducible population of stem cells in a cell culture media;
concurrently inducing direct differentiation and/or transdifferentiation of the at least one genetically-engineered inducible population of stem cells into at least two divergent populations of the differentiated or transdifferentiated programmable multicellular organoid and/or 3D organ-specific tissue cells, wherein the inducing step is independent of any external cues provided by the cell culture media; and
thereby forming the programmable multicellular organoid and/or a 3D organ-specific tissue comprising the at least two divergent populations of differentiated or transdifferentiated programmable multicellular organoid and/or 3D organ-specific tissue cells.

2. The method of claim 1, wherein the at least one genetically-engineered inducible population of stem cells comprises stem cells selected from the group consisting of pluripotent stem cells, multipotent stem cells, progenitor cells, terminally differentiated cells, endothelial cells, endothelial progenitor cells, immortalized cell lines, or primary cells.

3. The method of claim 1, wherein the at least one genetically-engineered inducible population of stem cells is created by introducing a DNA delivery element comprising at least one of constitutive promoter, small molecule inducible promoter, cell-autonomous promoter, cell non-autonomous promoter, selection marker, or a combination thereof.

4. The method of claim 1, wherein the genetically-engineered inducible population of stem cells overexpresses at least one transcription factor.

5. The method of claim 4, wherein the at least one transcription factor is selected from the group consisting of ETV2, NGNl, Tbr1, Fezf2, Ctip2, SATB2, LMXIA, NR4A2, lsI1, StI8, FOXA2, PITX3, AscII, Smad7, Nr2fl, Dlx2, Dlx4, Nr2f2, Barh12, and Lhx1.

6. The method of claim 1, wherein the differentiation and/or transdifferentiation of the of the at least one genetically-engineered inducible population of stem cells is induced via the addition or removal of small molecules, growth factors, dissolved gases, or morphogens to or from the cell culture media.

7. The method of claim 1, wherein the differentiation and/or transdifferentiation of the at least one genetically-engineered inducible population of stem cells is induced via the addition of doxycycline (DOX) into the cell culture media.

8. The method of claim 1, wherein the step of culturing is in a differentiation medium comprising doxycycline (DOX).

9. (canceled)

10. The method of claim 1, further comprising culturing a wild-type population of cells, and inducing differentiation of the wild-type population of cells into a different population of the programmable multicellular organoid and/or 3D organ-specific tissue cells.

11. The method of claim 10, wherein the differentiation of the wild-type population of cells is induced via the addition or removal of small molecules, growth factors, dissolved gases, or morphogens to or from the cell culture media.

12. The method of claim 1, wherein the step of culturing comprises culturing at least two genetically-engineered inducible populations of stem cells in the cell culture media.

13. The method of claim 1, wherein the method is used to enable the tailoring of the initial ratio and/or composition of pluripotent cell populations to deterministically define the different cell types and quantity within the resulting 3D human tissue.

14. A programmable multicellular organoid and/or a 3D organ-specific tissue produced by the method of claim 1.

15. An in vitro method of generating functional human tissue construct, the method comprising:
embedding a programmable multicellular organoid and/or a 3D organ-specific tissue in a tissue construct, the tissue construct comprising a first vascular network and a second vascular network, each vascular network comprising one or more interconnected vascular channels;
exposing the programmable multicellular organoid and/or a 3D organ-specific tissue to one or more biological agents, a biological agent gradient, a pressure, a pressure gradient, and/or an oxygen tension gradient, thereby inducing angiogenesis of capillary vessels to and/or from the programmable multicellular organoid and/or a 3D organ-specific tissue; and
wherein the exposing step promotes vascularizing the programmable multicellular organoid and/or a 3D organ-specific tissue, the capillary vessels connecting the first vascular network to the second vascular network, thereby creating a functional human tissue construct comprising a single vascular network and a perfusable tissue structure;
wherein the programmable multicellular organoid and/or a 3D organ-specific tissue is produced by_{:}
culturing at least one genetically-engineered inducible population of stem cells in a cell culture media;
concurrently inducing direct differentiation and/or transdifferentiation of the at least one genetically-engineered inducible population of stem cells into at least two divergent populations of the differentiated or transdifferentiated programmable multicellular organoid and/or 3D organ-specific tissue cells, wherein the inducing step is independent of any external cues provided by the cell culture media; and
thereby forming the programmable multicellular organoid and/or a 3D organ-specific tissue comprising the at least two divergent populations of differentiated or transdifferentiated programmable multicellular organoid and/or 3D organ-specific tissue cells.

16. The method of claim 15, wherein the culturing step comprises culturing a genetically-engineered inducible population of at least one of: pluripotent stem cells, multipotent stem cells, progenitor cells, terminally differentiated cells, endothelial cells, endothelial progenitor cells, immortalized cell lines, or primary cells in a cell differentiation media.

17. (canceled)

18. The method of claim 15, wherein the one or more biological agents, the biological agent gradient, the pressure, the pressure gradient, and/or the oxygen tension gradient further direct development, differentiation, and/or functioning of the programmable multicellular organoid and/or a 3D organ-specific tissue.

19. The method of claim 15, wherein the programmable multicellular organoid and/or a 3D organ-specific tissue is selected from the group consisting of: cerebral organoid or tissue, thyroid organoid or tissue, intestinal or gut organoid or tissue, hepatic organoid or tissue, pancreatic organoid or tissue, gastric organoid or tissue, kidney organoid or tissue, retinal organoid or tissue, cardiac organoid or tissue, bone organoid or tissue, and epithelial organoid or tissue.

20. The method of claim 15, wherein the programmable multicellular organoid and/or a 3D organ-specific tissue is exposed to the one or more biological agents and/or the biological agent gradient by at least one of:
diffusion of one or more biological agents within the tissue construct;
localized deposition of materials loaded with one or more biological agents within the tissue construct;
localized de-novo production of growth factors by localized protein translation; or
perfusion of one or both of the first and second vascular networks with one or more biological agents,
wherein the biological agents comprise one or more of the following: growth factors, morphogens, small molecules, drugs, hormones, DNA, shRNA, siRNA, nanoparticles, mRNA, modified mRNA;
wherein the growth factors comprise one or more of the following: vascular endothelial growth factor (VEGF), basic fibroblast growth factor (bFGF), sphingosine-1-phosphate (SIP), phorbol myristate acetate (PMA), hepatocyte growth factor (HGF), monocyte chemotactic protein-I (MCP-I), the angiopoietin ANG-I, the angiopoietin ANG-2, transforming growth factor beta (TGF-β), epidermal growth factor (EGF), human growth factor, matrix metalloproteinases (MMP’s), or histamine.

21. (canceled)

22. (canceled)

23. The method of claim 15, wherein the one or more interconnected vascular channels are formed by a manufacturing process or by a biological developmental process that may include at least one of vasculogenesis, angiogenesis, or tubulogenesis.

24. The method of claim 15, wherein the first vascular network and the second vascular network are independently addressable.

25. The method of claim 15, wherein the first vascular network and the second vascular network are not in contact with each other prior to the vascularizing step (c).

26. The method of claim 15, wherein the first vascular network comprises an arterial plexus and the second vascular network comprises a venous plexus.

27. The method of
(i) wherein the single vascular network comprises at least one of an interpenetrating vascular network or a branched interpenetrating vascular network; or
(ii) wherein the single vascular network comprises interconnected arterial and venous channels.

28. (canceled)

29. The method of claim 15,
wherein only one of the first and second vascular networks is perfused with the one or more biological agents prior to the vascularizing step (c); or
(ii) wherein both the first and second vascular networks are perfused with the one or more biological agents, and wherein a biological agent concentration in the first vascular network is different than a biological agent concentration in the second vascular network: or
(iii) wherein both the first and second vascular networks are perfused with the one or more biological agents, and wherein a biological agent concentration in the first vascular network is the same as a biological agent concentration in the second vascular network.

30. (canceled)

31. (canceled)

32. The method of claim 15, wherein an oxygen partial pressure gradient is introduced to one or both of the first and second vascular networks during perfusion.

33. The method of claim 15, wherein embedding the programmable multicellular organoid and/or a 3D organ-specific tissue in the tissue construct comprises:
depositing one or more cell-laden filaments each comprising a plurality of viable cells on a substrate to form one or more tissue patterns, each of the tissue patterns comprising one or more predetermined cell types;
depositing one or more sacrificial filaments on the substrate to form a vascular pattern interpenetrating the one or more tissue patterns, each of the sacrificial filaments comprising a fugitive ink;
depositing the programmable multicellular organoid and/or a 3D organ-specific tissue within the vascular pattern;
at least partially surrounding the one or more tissue patterns and the vascular pattern with an extracellular matrix composition; and
removing the fugitive ink, thereby forming the tissue construct comprising the programmable multicellular organoid and/or a 3D organ-specific tissue embedded therein.

34. An in vitro method of generating functional human tissue construct comprising:
depositing one or more cell-laden filaments each comprising a bioink comprising at least one genetically-engineered inducible population of stem cells on a substrate or into a supporting matrix, to form one or more tissue patterns, each of the tissue patterns comprising at least one predetermined genetically-engineered inducible population of stem cells;
depositing one or more sacrificial filaments on the substrate to form a vascular pattern;
at least partially surrounding the one or more tissue patterns and the vascular pattern with an extracellular matrix composition; and
removing the fugitive ink, thereby forming the functional tissue construct comprising tissue patterns comprising at least one predetermined genetically-engineered inducible population of stem cells embedded therein.

35. The method of claim 34, further comprising inducing direct differentiation and/or transdifferentiation of the at least one genetically-engineered inducible population of stem cells into at least two divergent populations of the programmable multicellular organoid and/or 3D organ-specific tissue cells.

36. The method of claim 35, wherein the differentiation and/or transdifferentiation of the at least one genetically-engineered inducible population of stem cells is induced via the addition of doxycycline (DOX) into the cell culture media.

37. The method of claim 34, wherein the at least one genetically-engineered inducible population of stem cells overexpresses at least one transcription factor selected from the group consisting of ETV2, NGNI, TbrI, Fezf2, Ctip2, SATB2, LMXIA, NR4A2, lsI1, St18, FOXA2, PlTX3, AscI1, Smad7, Nr2fl, Dlx2, Dlx4, Nr2f2, Barhl2, and Lhx1.

38. (canceled)

39. The method of claim 34,wherein the bioink comprises at least two genetically-engineered inducible populations of stem cells.

40. The method of claim 34,wherein the at least one genetically-engineered inducible population of stem cells is genomically programmed using an orthogonally induced differentiation platform.

41. The method of claim 34, wherein the bioink comprises the at least one genetically-engineered inducible population of stem cells at cell density of at least 100 M cells/mL.

42. The method of claim 34, wherein the bioink is composed of a cellular pellet comprising the at least one genetically-engineered inducible population of stem cells, formed via centrifugation of a cellular suspension and removal of the supernatant.

43. The method of claim 42, wherein extracellular matrix components or rheological modifiers are optionally added to the cellular suspension prior to centrifugation.

44. (canceled)

45. (canceled)
